SEQUENCE LISTING (35 sequences)

SEQ ID NO: 1 - protein, 1368 aa, Streptococcus pyogenes [sequence omitted]

SEQ ID NO: 2 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1121 aa, Streptococcus thermophilus
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINDKGKEVPCNPFLKYKEEHGYIRKYSKKGNGPEIKSLKYYDSKLGNHIDITPKDSNNKVVLQSVSPWRADVYFNKTTGKYEILGLKYADLQFDKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 4 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 5 - protein, 1037 aa, Parvibaculum lavamentivorans
MERIFGFDIGTTSIGFSVIDYSSTQSAGNIQRLGVRIFPEARDPDGTPLNQQRRQKRMMRRQLRRRRIRRKALNETLHEAGFLPAYGSADWPVVMADEPYELRRRGLEEGLSAYEFGRAIYHLAQHRHFKGRELEESDTPDPDVDDEKEAANERAATLKALKNEQTTLGAWLARRPPSDRKRGIHAHRNVVAEEFERLWEVQSKFHPALKSEEMRARISDTIFAQRPVFWRKNTLGECRFMPGEPLCPKGSWLSQQRRMLEKLNNLAIAGGNARPLDAEERDAILSKLQQQASMSWPGVRSALKALYKQRGEPGAEKSLKFNLELGGESKLLGNALEAKLADMFGPDWPAHPRKQEIRHAVHERLWAADYGETPDKKRVIILSEKDRKAHREAAANSFVADFGITGEQAAQLQALKLPTGWEPYSIPALNLFLAELEKGERFGALVNGPDWEGWRRTNFPHRNQPTGEILDKLPSPASKEERERISQLRNPTVVRTQNELRKVVNNLIGLYGKPDRIRIEVGRDVGKSKREREEIQSGIRRNEKQRKKATEDLIKNGIANPSRDDVEKWILWKEGQERCPYTGDQIGFNALFREGRYEVEHIWPRSRSFDNSPRNKTLCRKDVNIEKGNRMPFEAFGHDEDRWSAIQIRLQGMVSAKGGTGMSPGKVKRFLAKTMPEDFAARQLNDTRYAAKQILAQLKRLWPDMGPEAPVKVEAVTGQVTAQLRKLWTLNNILADDGEKTRADHRHHAIDALTVACTHPGMTNKLSRYWQLRDDPRAEKPALTPPWDTIRADAEKAVSEIVVSHRVRKKVSGPLHKETTYGDTGTDIKTKSGTYRQFVTRKKIESLSKGELDEIRDPRIKEIVAAHVAGRGGDPKKAFPPYPCVSPGGPEIRKVRLTSKQQLNLMAQTGNGYADLGSNHHIAIYRLPDGKADFEIVSLFDASRRLAQRNPIVQRTRADGASFVMSLAAGEAIMIPEGSKKGIWIVQGVWASGQVVLERDTDADHSTTTRPMPNPILKDDAKKVSIDPIGRVRPSND

SEQ ID NO: 6 - protein, 1084 aa, Corynebacter diphtheria
MKYHVGIDVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 7 - protein, 1130 aa, Streptococcus pasteurianus
MTNGKILGLDIGIASVGVGIIEAKTGKVVHANSRLFSAANAENNAERRGFRGSRRLNRRKKHRVKRVRDLFEKYGIVTDFRNLNLNPYELRVKGLTEQLKNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVSTETGKLSTEQKESLVEFAKNTATLGPAKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGNKQLETKIRLWYQQGERCLYSGKPISIQELVHNSNNFEIDHILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKVSVVRGQFTSQLRRKWKIDKSRETYHHHAVDALIIAASSQLKLWEKQDNPMFVDYGKNQVVDKQTGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDSLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGNYHISQEKYDAIKEKEGIGKKSEFKFTLYRNDLILIKDIASGEQEIYRFLSRTMPNVNHYVELKPYDKEKFDNVQELVEALGEADKVGRCIKGLNKPNISIYKVRTDVLGNKYFVKKKGDKPKLDFKNNKK

SEQ ID NO: 8 - protein, 1082 aa, Neisseria cinerea
MAAFKPNPMNYILGLDIGIASVGWAIVEIDEEENPIRLIDLGVRVFERAEVPKTGDSLAAARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNTHALQTGDFRTPAELALNKFEKESGHIRNQRGDYSHTFNRKDLQAELNLLFEKQKEFGNPHVSDGLKEGIETLLMTQRPALSGDAVQKMLGHCTFEPTEPKAAKNTYTAERFVWLTKLNNLRILEQGSERPLTDTERATLMDEPYRKSKLTYAQARKLLDLDDTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGNRYDEACTEIYGDHYGKKNTEEKIYLPPIPADEIRNPVVLRALSQARKVINGVVRRYGSPARIHIETAREVGKSFKDRKEIEKRQEENRKDREKSAAKFREYFPNFVGEPKSKDILKLRLYEQQHGKCLYSGKEINLGRLNEKGYVEIDHALPFSRTWDDSFNNKVLALGSENQNKGNQTPYEYFNGKDNSREWQEFKARVETSRFPRSKKQRILLQKFDEDGFKERNLNDTRYINRFLCQFVADHMLLTGKGKRRVFASNGQITNLLRGFWGLRKVRAENDRHHALDAVVVACSTIAMQQKITRFVRYKEMNAFDGKTIDKETGEVLHQKAHFPQPWEFFAQEVMIRVFGKPDGKPEFEEADTPEKLRTLLAEKLSSRPEAVHKYVTPLFISRAPNRKMSGQGHMETVKSAKRLDEGISVLRVPLTQLKLKDLEKMVNREREPKLYEALKARLEAHKDDPAKAFAEPFYKYDKAGNRTQQVKAVRVEQVQKTGVWVHNHNGIADNATIVRVDVFEKGGKYYLVPIYSWQVAKGILPDRAVVQGKDEEDWTVMDDSFEFKFVLYANDLIKLTAKKNEFLGYFVSLNRATGAIDIRTHDTDSTKGKNGIFQSVGVKTALSFQKYQIDELGKEIRPCRLKKRPPVR

SEQ ID NO: 9 - protein, 1003 aa, Campylobacter lari
MRILGFDIGINSIGWAFVENDELKDCGVRIFTKAENPKNKESLALPRRNARSSRRRLKRRKARLIAIKRILAKELKLNYKDYVAADGELPKAYEGSLASVYELRYKALTQNLETKDLARVILHIAKHRGYMNKNEKKSNDAKKGKILSALKNNALKLENYQSVGEYFYKEFFQKYKKNTKNFIKIRNTKDNYNNCVLSSDLEKELKLILEKQKEFGYNYSEDFINEILKVAFFQRPLKDFSHLVGACTFFEEEKRACKNSYSAWEFVALTKIINEIKSLEKISGEIVPTQTINEVLNLILDKGSITYKKFRSCINLHESISFKSLKYDKENAENAKLIDFRKLVEFKKALGVHSLSRQELDQISTHITLIKDNVKLKTVLEKYNLSNEQINNLLEIEFNDYINLSFKALGMILPLMREGKRYDEACEIANLKPKTVDEKKDFLPAFCDSIFAHELSNPVVNRAISEYRKVLNALLKKYGKVHKIHLELARDVGLSKKAREKIEKEQKENQAVNAWALKECENIGLKASAKNILKLKLWKEQKEICIYSGNKISIEHLKDEKALEVDHIYPYSRSFDDSFINKVLVFTKENQEKLNKTPFEAFGKNIEKWSKIQTLAQNLPYKKKNKILDENFKDKQQEDFISRNLNDTRYIATLIAKYTKEYLNFLLLSENENANLKSGEKGSKIHVQTISGMLTSVLRHTWGFDKKDRNNHLHHALDAIIVAYSTNSIIKAFSDFRKNQELLKARFYAKELTSDNYKHQVKFFEPFKSFREKILSKIDEIFVSKPPRKRARRALHKDTFHSENKIIDKCSYNSKEGLQIALSCGRVRKIGTKYVENDTIVRVDIFKKQNKFYAIPIYAMDFALGILPNKIVITGKDKNNNPKQWQTIDESYEFCFSLYKNDLILLQKKNMQEPEFAYYNDFSISTSSICVEKHDNKFENLTSNQKLLFSNAKEGSVKVESLGIQNLKVFEKYIITPLGDKIKADFQPRENISLKTSKKYGLR

SEQ ID NO: 10 - protein, 1395 aa, Treponema denticola
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV

SEQ ID NO: 11 - protein, 1345 aa, Streptococcus mutans
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 12 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 13 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 14 - protein, 1056 aa, Pasteurella multocida
MQTTNLSYILGLDLGIASVGWAVVEINENEDPIGLIDVGVRIFERAEVPKTGESLALSRRLARSTRRLIRRRAHRLLLAKRFLKREGILSTIDLEKGLPNQAWELRVAGLERRLSAIEWGAVLLHLIKHRGYLSKRKNESQTNNKELGALLSGVAQNHQLLQSDDYRTPAELALKKFAKEEGHIRNQRGAYTHTFNRLDLLAELNLLFAQQHQFGNPHCKEHIQQYMTELLMWQKPALSGEAILKMLGKCTHEKNEFKAAKHTYSAERFVWLTKLNNLRILEDGAERALNEEERQLLINHPYEKSKLTYAQVRKLLGLSEQAIFKHLRYSKENAESATFMELKAWHAIRKALENQGLKDTWQDLAKKPDLLDEIGTAFSLYKTDEDIQQYLTNKVPNSVINALLVSLNFDKFIELSLKSLRKILPLMEQGKRYDQACREIYGHHYGEANQKTSQLLPAIPAQEIRNPVVLRTLSQARKVINAIIRQYGSPARVHIETGRELGKSFKERREIQKQQEDNRTKRESAVQKFKELFSDFSSEPKSKDILKFRLYEQQHGKCLYSGKEINIHRLNEKGYVEIDHALPFSRTWDDSFNNKVLVLASENQNKGNQTPYEWLQGKINSERWKNFVALVLGSQCSAAKKQRLLTQVIDDNKFIDRNLNDTRYIARFLSNYIQENLLLVGKNKKNVFTPNGQITALLRSRWGLIKARENNNRHHALDAIVVACATPSMQQKITRFIRFKEVHPYKIENRYEMVDQESGEIISPHFPEPWAYFRQEVNIRVFDNHPDTVLKEMLPDRPQANHQFVQPLFVSRAPTRKMSGQGHMETIKSAKRLAEGISVLRIPLTQLKPNLLENMVNKEREPALYAGLKARLAEFNQDPAKAFATPFYKQGGQQVKAIRVEQVQKSGVLVRENNGVADNASIVRTDVFIKNNKFFLVPIYTWQVAKGILPNKAIVAHKNEDEWEEMDEGAKFKFSLFPNDLVELKTKKEYFFGYYIGLDRATGNISLKEHDGEISKGKDGVYRVGVKLALSFEKYQVDELGKNRQICRPQQRQPVR

SEQ ID NO: 15 - protein, 1629 aa, Francisella novicida
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 16 - protein, 1371 aa, Lactobacillus buchneri
MKVNNYHIGLDIGTSSIGWVAIGKDGKPLRVKGKTAIGARLFQEGNPAADRRMFRTTRRRLSRRKWRLKLLEEIFDPYITPVDSTFFARLKQSNLSPKDSRKEFKGSMLFPDLTDMQYHKNYPTIYHLRHALMTQDKKFDIRMVYLAIHHIVKYRGNFLNSTPVDSFKASKVDFVDQFKKLNELYAAINPEESFKINLANSEDIGHQFLDPSIRKFDKKKQIPKIVPVMMNDKVTDRLNGKIASEIIHAILGYKAKLDVVLQCTPVDSKPWALKFDDEDIDAKLEKILPEMDENQQSIVAILQNLYSQVTLNQIVPNGMSLSESMIEKYNDHHDHLKLYKKLIDQLADPKKKAVLKKAYSQYVGDDGKVIEQAEFWSSVKKNLDDSELSKQIMDLIDAEKFMPKQRTSQNGVIPHQLHQRELDEIIEHQSKYYPWLVEINPNKHDLHLAKYKIEQLVAFRVPYYVGPMITPKDQAESAETVFSWMERKGTETGQITPWNFDEKVDRKASANRFIKRMTTKDTYLIGEDVLPDESLLYEKFKVLNELNMVRVNGKLLKVADKQAIFQDLFENYKHVSVKKLQNYIKAKTGLPSDPEISGLSDPEHFNNSLGTYNDFKKLFGSKVDEPDLQDDFEKIVEWSTVFEDKKILREKLNEITWLSDQQKDVLESSRYQGWGRLSKKLLTGIVNDQGERIIDKLWNTNKNFMQIQSDDDFAKRIHEANADQMQAVDVEDVLADAYTSPQNKKAIRQVVKVVDDIQKAMGGVAPKYISIEFTRSEDRNPRRTISRQRQLENTLKDTAKSLAKSINPELLSELDNAAKSKKGLTDRLYLYFTQLGKDIYTGEPINIDELNKYDIDHILPQAFIKDNSLDNRVLVLTAVNNGKSDNVPLRMFGAKMGHFWKQLAEAGLISKRKLKNLQTDPDTISKYAMHGFIRRQLVETSQVIKLVANILGDKYRNDDTKIIEITARMNHQMRDEFGFIKNREINDYHHAFDAYLTAFLGRYLYHRYIKLRPYFVYGDFKKFREDKVTMRNFNFLHDLTDDTQEKIADAETGEVIWDRENSIQQLKDVYHYKFMLISHEVYTLRGAMFNQTVYPASDAGKRKLIPVKADRPVNVYGGYSGSADAYMAIVRIHNKKGDKYRVVGVPMRALDRLDAAKNVSDADFDRALKDVLAPQLTKTKKSRKTGEITQVIEDFEIVLGKVMYRQLMIDGDKKFMLGSSTYQYNAKQLVLSDQSVKTLASKGRLDPLQESMDYNNVYTEILDKVNQYFSLYDMNKFRHKLNLGFSKFISFPNHNVLDGNTKVSSGKREILQEILNGLHANPTFGNLKDVGITTPFGQLQQPNGILLSDETKIRYQSPTGLFERTVSLKDL

SEQ ID NO: 17 - protein, 1334 aa, Listeria innocua
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 18 - protein, 1372 aa, Legionella pneumophila
MESSQILSPIGIDLGGKFTGVCLSHLEAFAELPNHANTKYSVILIDHNNFQLSQAQRRATRHRVRNKKRNQFVKRVALQLFQHILSRDLNAKEETALCHYLNNRGYTYVDTDLDEYIKDETTINLLKELLPSESEHNFIDWFLQKMQSSEFRKILVSKVEEKKDDKELKNAVKNIKNFITGFEKNSVEGHRHRKVYFENIKSDITKDNQLDSIKKKIPSVCLSNLLGHLSNLQWKNLHRYLAKNPKQFDEQTFGNEFLRMLKNFRHLKGSQESLAVRNLIQQLEQSQDYISILEKTPPEITIPPYEARTNTGMEKDQSLLLNPEKLNNLYPNWRNLIPGIIDAHPFLEKDLEHTKLRDRKRIISPSKQDEKRDSYILQRYLDLNKKIDKFKIKKQLSFLGQGKQLPANLIETQKEMETHFNSSLVSVLIQIASAYNKEREDAAQGIWFDNAFSLCELSNINPPRKQKILPLLVGAILSEDFINNKDKWAKFKIFWNTHKIGRTSLKSKCKEIEEARKNSGNAFKIDYEEALNHPEHSNNKALIKIIQTIPDIIQAIQSHLGHNDSQALIYHNPFSLSQLYTILETKRDGFHKNCVAVTCENYWRSQKTEIDPEISYASRLPADSVRPFDGVLARMMQRLAYEIAMAKWEQIKHIPDNSSLLIPIYLEQNRFEFEESFKKIKGSSSDKTLEQAIEKQNIQWEEKFQRIINASMNICPYKGASIGGQGEIDHIYPRSLSKKHFGVIFNSEVNLIYCSSQGNREKKEEHYLLEHLSPLYLKHQFGTDNVSDIKNFISQNVANIKKYISFHLLTPEQQKAARHALFLDYDDEAFKTITKFLMSQQKARVNGTQKFLGKQIMEFLSTLADSKQLQLEFSIKQITAEEVHDHRELLSKQEPKLVKSRQQSFPSHAIDATLTMSIGLKEFPQFSQELDNSWFINHLMPDEVHLNPVRSKEKYNKPNISSTPLFKDSLYAERFIPVWVKGETFAIGFSEKDLFEIKPSNKEKLFTLLKTYSTKNPGESLQELQAKSKAKWLYFPINKTLALEFLHHYFHKEIVTPDDTTVCHFINSLRYYTKKESITVKILKEPMPVLSVKFESSKKNVLGSFKHTIALPATKDWERLFNHPNFLALKANPAPNPKEFNEFIRKYFLSDNNPNSDIPNNGHNIKPQKHKAVRKVFSLPVIPGNAGTMMRIRRKDNKGQPLYQLQTIDDTPSMGIQINEDRLVKQEVLMDAYKTRNLSTIDGINNSEGQAYATFDNWLTLPVSTFKPEIIKLEMKPHSKTRRYIRITQSLADFIKTIDEALMIKPSDSIDDPLNMPNEIVCKNKLFGNELKPRDGKMKIVSTGKIVTYEFESDSTPQWIQTLYVTQLKKQP

SEQ ID NO: 19 - protein, 1082 aa, Neisseria lactamica
MAAFKPNPMNYILGLDIGIASVGWAMVEVDEEENPIRLIDLGVRVFERAEVPKTGDSLAMARRLARSVRRLTRRRAHRLLRARRLLKREGVLQDADFDENGLVKSLPNTPWQLRAAALDRKLTCLEWSAVLLHLVKHRGYLSQRKNEGETADKELGALLKGVADNAHALQTGDFRTPAELALNKFEKESGHIRNQRGDYSHTFSRKDLQAELNLLFEKQKEFGNPHVSDGLKEDIETLLMAQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSTELQDEIGTAFSLFKTDKDITGRLKDRVQPEILEALLKHISFDKFVQISLKALRRIVPLMEQGKRYDEACAEIYGDHYCKKNAEEKIYLPPIPADEIRNPVVLRALSQARKVINCVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEEGFKERNLNDTRYVNRFLCQFVADHILLTGKGKRRVFASNGQITNLLRGFWGLRKVRTENDRHHALDAVVVACSTVAMQQKITRFVRYKEMNAFDGKTIDKETGEVLHQKAHFPQPWEFFAQEVMIRVFGKPDGKPEFEEADTPEKLRTLLAEKLSSRPEAVHEYVTPLFVSRAPNRKMSGQGHMETVKSAKRLDEGISVLRVPLTQLKLKGLEKMVNREREPKLYDALKAQLETHKDDPAKAFAEPFYKYDKAGSRTQQVKAVRIEQVQKTGVWVRNHNGIADNATMVRVDVFEKGGKYYLVPIYSWQVAKGILPDRAVVAFKDEEDWTVMDDSFEFRFVLYANDLIKLTAKKNEFLGYFVSLNRATGAIDIRTHDTDSTKGKNGIFQSVGVKTALSFQKNQIDELGKEIRPCRLKKRPPVR

SEQ ID NO: 20 - protein, 1082 aa, Neisseria meningitidis; identical to SEQ ID NO: 4

SEQ ID NO: 21 - protein, 1187 aa, Bifidobacterium longum
MLSRQLLGASHLARPVSYSYNVQDNDVHCSYGERCFMRGKRYRIGIDVGLNSVGLAAVEVSDENSPVRLLNAQSVIHDGGVDPQKNKEAITRKNMSGVARRTRRMRRRKRERLHKLDMLLGKFGYPVIEPESLDKPFEEWHVRAELATRYIEDDELRRESISIALRHMARHRGWRNPYRQVDSLISDNPYSKQYGELKEKAKAYNDDATAAEEESTPAQLVVAMLDAGYAEAPRLRWRTGSKKPDAEGYLPVRLMQEDNANELKQIFRVQRVPADEWKPLFRSVFYAVSPKGSAEQRVGQDPLAPEQARALKASLAFQEYRIANVITNLRIKDASAELRKLTVDEKQSIYDQLVSPSSEDITWSDLCDFLGFKRSQLKGVGSLTEDGEERISSRPPRLTSVQRIYESDNKIRKPLVAWWKSASDNEHEAMIRLLSNTVDIDKVREDVAYASAIEFIDGLDDDALTKLDSVDLPSGRAAYSVETLQKLTRQMLTTDDDLHEARKTLFNVTDSWRPPADPIGEPLGNPSVDRVLKNVNRYLMNCQQRWGNPVSVNIEHVRSSFSSVAFARKDKREYEKNNEKRSIFRSSLSEQLRADEQMEKVRESDLRRLEAIQRQNGQCLYCGRTITFRTCEMDHIVPRKGVGSTNTRTNFAAVCAECNRMKSNTPFAIWARSEDAQTRGVSLAEAKKRVTMFTFNPKSYAPREVKAFKQAVIARLQQTEDDAAIDNRSIESVAWMADELHRRIDWYFNAKQYVNSASIDDAEAETMKTTVSVFQGRVTASARRAAGIEGKIHFIGQQSKTRLDRRHHAVDASVIAMMNTAAAQTLMERESLRESQRLIGLMPGERSWKEYPYEGTSRYESFHLWLDNMDVLLELLNDALDNDRIAVMQSQRYVLGNSIAHDATIHPLEKVPLGSAMSADLIRRASTPALWCALTRLPDYDEKEGLPEDSHREIRVHDTRYSADDEMGFFASQAAQIAVQEGSADIGSAIHHARVYRCWKTNAKGVRKYFYGMIRVFQTDLLRACHDDLFTVPLPPQSISMRYGEPRVVQALQSGNAQYLGSLVVGDEIEMDFSSLDVDGQIGEYLQFFSQFSGGNLAWKHWVVDGFFNQTQLRIRPRYLAAEGLAKAFSDDVVPDGVQKIVTKQGWLPPVNTASKTAVRIVRRNAFGEPRLSSAHHMPCSWQWRHE

SEQ ID NO: 22 - protein, 1101 aa, Akkermansia muciniphila
MSRSLTFSFDIGYASIGWAVIASASHDDADPSVCGCGTVLFPKDDCQAFKRREYRRLRRNIRSRRVRIERIGRLLVQAQIITPEMKETSGHPAPFYLASEALKGHRTLAPIELWHVLRWYAHNRGYDNNASWSNSLSEDGGNGEDTERVKHAQDLMDKHGTATMAETICRELKLEEGKADAPMEVSTPAYKNLNTAFPRLIVEKEVRRILELSAPLIPGLTAEIIELIAQHHPLTTEQRGVLLQHGIKLARRYRGSLLFGQLIPRFDNRIISRCPVTWAQVYEAELKKGNSEQSARERAEKLSKVPTANCPEFYEYRMARILCNIRADGEPLSAEIRRELMNQARQEGKLTKASLEKAISSRLGKETETNVSNYFTLHPDSEEALYLNPAVEVLQRSGIGQILSPSVYRIAANRLRRGKSVTPNYLLNLLKSRGESGEALEKKIEKESKKKEADYADTPLKPKYATGRAPYARTVLKKVVEEILDGEDPTRPARGEAHPDGELKAHDGCLYCLLDTDSSVNQHQKERRLDTMTNNHLVRHRMLILDRLLKDLIQDFADGQKDRISRVCVEVGKELTTFSAMDSKKIQRELTLRQKSHTDAVNRLKRKLPGKALSANLIRKCRIAMDMNWTCPFTGATYGDHELENLELEHIVPHSFRQSNALSSLVLTWPGVNRMKGQRTGYDFVEQEQENPVPDKPNLHICSLNNYRELVEKLDDKKGHEDDRRRKKKRKALLMVRGLSHKHQSQNHEAMKEIGMTEGMMTQSSHLMKLACKSIKTSLPDAHIDMIPGAVTAEVRKAWDVFGVFKELCPEAADPDSGKILKENLRSLTHLHHALDACVLGLIPYIIPAHHNGLLRRVLAMRRIPEKLIPQVRPVANQRHYVLNDDGRMMLRDLSASLKENIREQLMEQRVIQHVPADMGGALLKETMQRVLSVDGSGEDAMVSLSKKKDGKKEKNQVKASKLVGVFPEGPSKLKALKAAIEIDGNYGVALDPKPVVIRHIKVFKRIMALKEQNGGKPVRILKKGMLIHLTSSKDPKHAGVWRIESIQDSKGGVKLDLQRAHCAVPKNKTHECNWREVDLISLLKKYQMKRYPTSYTGTPR

SEQ ID NO: 23 - protein, 1498 aa, Odoribacter laneus
METTLGIDLGTNSIGLALVDQEEHQILYSGVRIFPEGINKDTIGLGEKEESRNATRRAKRQMRRQYFRKKLRKAKLLELLIAYDMCPLKPEDVRRWKNWDKQQKSTVRQFPDTPAFREWLKQNPYELRKQAVTEDVTRPELGRILYQMIQRRGFLSSRKGKEEGKIFTGKDRMVGIDETRKNLQKQTLGAYLYDIAPKNGEKYRFRTERVRARYTLRDMYIREFEIIWQRQAGHLGLAHEQATRKKNIFLEGSATNVRNSKLITHLQAKYGRGHVLIEDTRITVTFQLPLKEVLGGKIEIEEEQLKFKSNESVLFWQRPLRSQKSLLSKCVFEGRNFYDPVHQKWIIAGPTPAPLSHPEFEEFRAYQFINNIIYGKNEHLTAIQREAVFELMCTESKDFNFEKIPKHLKLFEKFNFDDTTKVPACTTISQLRKLFPHPVWEEKREEIWHCFYFYDDNTLLFEKLQKDYALQTNDLEKIKKIRLSESYGNVSLKAIRRINPYLKKGYAYSTAVLLGGIRNSFGKRFEYFKEYEPEIEKAVCRILKEKNAEGEVIRKIKDYLVHNRFGFAKNDRAFQKLYHHSQAITTQAQKERLPETGNLRNPIVQQGLNELRRTVNKLLATCREKYGPSFKFDHIHVEMGRELRSSKTEREKQSRQIRENEKKNEAAKVKLAEYGLKAYRDNIQKYLLYKEIEEKGGTVCCPYTGKTLNISHTLGSDNSVQIEHIIPYSISLDDSLANKTLCDATFNREKGELTPYDFYQKDPSPEKWGASSWEEIEDRAFRLLPYAKAQRFIRRKPQESNEFISRQLNDTRYISKKAVEYLSAICSDVKAFPGQLTAELRHLWGLNNILQSAPDITFPLPVSATENHREYYVITNEQNEVIRLFPKQGETPRTEKGELLLTGEVERKVFRCKGMQEFQTDVSDGKYWRRIKLSSSVTWSPLFAPKPISADGQIVLKGRIEKGVFVCNQLKQKLKTGLPDGSYWISLPVISQTFKEGESVNNSKLTSQQVQLFGRVREGIFRCHNYQCPASGADGNFWCTLDTDTAQPAFTPIKNAPPGVGGGQIILTGDVDDKGIFHADDDLHYELPASLPKGKYYGIFTVESCDPTLIPIELSAPKTSKGENLIEGNIWVDEHTGEVRFDPKKNREDQRHHAIDAIVIALSSQSLFQRLSTYNARRENKKRGLDSTEHFPSPWPGFAQDVRQSVVPLLVSYKQNPKTLCKISKTLYKDGKKIHSCGNAVRGQLHKETVYGQRTAPGATEKSYHIRKDIRELKTSKHIGKVVDITIRQMLLKHLQENYHIDITQEFNIPSNAFFKEGVYRIFLPNKHGEPVPIKKIRMKEELGNAERLKDNINQYVNPRNNHHVMIYQDADGNLKEEIVSFWSVIERQNQGQPIYQLPREGRNIVSILQINDTFLIGLKEEEPEVYRNDLSTLSKHLYRVQKLSGMYYTFRHHLASTLNNEREEFRIQSLEAWKRANPVKVQIDEIGRITFLNGPLC

SEQ ID NO: 24 - protein, 580 aa, Homo sapiens
MSDTWSSIQAHKKQLDSLRERLQRRRKQDSGHLDLRNPEAALSPTFRSDSPVPTAPTSGGPKPSTASAVPELATDPELEKKLLHHLSDLALTLPTDAVSICLAISTPDAPATQDGVESLLQKFAAQELIEVKRGLLQDDAHPTLVTYADHSKLSAMMGAVAEKKGPGEVAGTVTGQKRRAEQDSTTVAAFASSLVSGLNSSASEPAKEPAKKSRKHAASDVDLEIESLLNQQSTKEQQSKKVSQEILELLNTTTAKEQSIVEKFRSRGRAQVQEFCDYGTKEECMKASDADRPCRKLHFRRIINKHTDESLGDCSFLNTCFHMDTCKYVHYEIDACMDSEAPGSKDHTPSQELALTQSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 25 - DNA, 2038 bp, Homo sapiens
aaatgacttttctgtcttgctcagctccaggggtcattttccggttagccttcggggtgtccgcgtgagaattggctatatcctggagcgagtgctgggaggtgctagtccgccgcgccttattcgagaggtgtcagggctgggagactaggatgtcggacacgtggagctctatccaggcccacaagaagcagctggactctctgcgggagaggctgcagcggaggcggaagcaggactcggggcacttggatctacggaatccagaggcagcattgtctccaaccttccgtagtgacagcccagtgcctactgcacccacctctggtggccctaagcccagcacagcttcagcagttcctgaattagctacagatcctgagttagagaagaagttgctacaccacctctctgatctggccttaacattgcccactgatgctgtgtccatctgtcttgccatctccacgccagatgctcctgccactcaagatggggtagaaagcctcctgcagaagtttgcagctcaggagttgattgaggtaaagcgaggtctcctacaagatgatgcacatcctactcttgtaacctatgctgaccattccaagctctctgccatgatgggtgctgtggcagaaaagaagggccctggggaggtagcagggactgtcacagggcagaagcggcgtgcagaacaggactcgactacagtagctgcctttgccagttcgttagtctctggtctgaactcttcagcatcggaaccagcaaaggagccagccaagaaatcaaggaaacatgctgcctcagatgttgatctggagatagagagccttctgaaccaacagtccactaaggaacaacagagcaagaaggtcagtcaggagatcctagagctattaaatactacaacagccaaggaacaatccattgttgaaaaatttcgctctcgaggtcgggcccaagtgcaagaattctgtgactatggaaccaaggaggagtgcatgaaagccagtgatgctgatcgaccctgtcgcaagctgcacttcagacgaattatcaataaacacactgatgagtctttaggtgactgctctttccttaatacatgtttccacatggatacctgcaagtatgttcactatgaaattgatgcttgcatggattctgaggcccctggcagcaaagaccacacgccaagccaggagcttgctcttacacagagtgtcggaggtgattccagtgcagaccgactcttcccacctcagtggatctgttgtgatatccgctacctggacgtcagtatcttgggcaagtttgcagttgtgatggctgacccaccctgggatattcacatggaactgccctatgggaccctgacagatgatgagatgcgcaggctcaacatacccgtactacaggatgatggctttctcttcctctgggtcacaggcagggccatggagttggggagagaatgtctaaacctctgggggtatgaacgggtagatgaaattatttgggtgaagacaaatcaactgcaacgcatcattcggacaggccgtacaggtcactggttgaaccatgggaaggaacactgcttggttggtgtcaaaggaaatccccaaggcttcaaccagggtctggattgtgatgtgatcgtagctgaggttcgttccaccagtcataaaccagatgaaatctatggcatgattgaaagactatctcctggcactcgcaagattgagttatttggacgaccacacaatgtgcaacccaactggatcacccttggaaaccaactggatgggatccacctactagacccagatgtggttgcacggttcaagcaaaggtacccagatggtatcatctctaaacctaagaatttatagaagcacttccttacagagctaagaatccatagccatggctctgtaagctaaacctgaagagtgatatttgtacaatagctttcttctttatttaaataaacatttgtattgtagttgggattctgaaaaaaaaaaaaaaaaaa

SEQ ID NO: 26 - protein, 456 aa, Homo sapiens
MDSRLQEIRERQKLRRQLLAQQLGAESADSIGAVLNSKDEQREIAETRETCRASYDTSAPNAKRKYLDEGETDEDKMEEYKDELEMQQDEENLPYEEEIYKDSSTFLKGTQSLNPHNDYCQHFVDTGHRPQNFIRDVGLADRFEEYPKLRELIRLKDELIAKSNTPPMYLQADIEAFDIRELTPKFDVILLEPPLEEYYRETGITANEKCWTWDDIMKLEIDEIAAPRSFIFLWCGSGEGLDLGRVCLRKWGYRRCEDICWIKTNKNNPGKTKTLDPKAVFQRTKEHCLMGIKGTVKRSTDGDFIHANVDIDLIITEEPEIGNIEKPVEIFHIIEHFCLGRRRLHLFGRDSTIRPGWLTVGPTLTNSNYNAETYASYFSAPNSYLTGCTEEIERLRPKSPPPKSKSDRGGGAPRGGGRGGTSAGRGRERNRSNFRGERGGFRGGRGGAHRGGFPPR

SEQ ID NO: 27 - DNA, 3520 bp, Homo sapiens
gagccaattccggccgcgccggaagtctctactgaggaaagctatgaggatactctgttcgtaagctcccggtgaattttgttccacagactcggaagaaaggttggataagagttcactggagattgacaagtactcgggatagtgaaaagccggagttggaacatggatagccgcttgcaggagatccgggagcggcagaagttacggcgacagctcctcgcgcagcagttgggagctgaaagtgccgacagcattggtgccgtgttaaatagcaaagatgagcagagagaaattgctgaaacaagagaaacttgcagggcttcctatgatacctctgctccaaatgcaaaacgtaagtatctggatgaaggagagacagatgaagacaaaatggaagaatataaggatgaactagaaatgcaacaggatgaagaaaatttgccatatgaagaagagatttacaaagattctagtacttttcttaagggaacacagagcttaaatccccataatgattactgccaacattttgtagacactggacatagacctcagaatttcatcagggatgtaggtttagctgacagatttgaagaatatcctaaactgagggagctcatcaggctaaaggatgagttaatagctaaatctaacactcctcccatgtacttacaagccgatatagaagcctttgacatcagagaactaacacccaaatttgatgtgattcttctggaaccccctttagaagaatattacagagaaactggcatcactgctaatgaaaaatgctggacttgggatgatattatgaagttagaaattgatgagattgcagcacctcgatcatttatttttctctggtgtggttctggggaggggttggaccttggaagagtgtgtttacgaaaatggggttacagaagatgtgaagatatttgttggattaaaaccaataaaaacaatcctgggaagactaagactttagatccaaaggctgtctttcagagaacaaaggaacactgcctcatggggatcaaaggaactgtgaagcgtagcacagacggggacttcattcatgctaatgttgacattgacttaattatcacagaagaacctgaaattggcaatatagaaaaacctgtagaaatttttcatataattgagcatttttgtcttggtagaagacgccttcatctatttggaagagatagtacaattcgaccaggctggctcacagttggaccaacgcttacaaatagcaactacaatgcagaaacatatgcatcctatttcagtgctcctaattcctacttgactggttgtacagaagaaattgagagacttcgaccaaaatcgcctcctcccaaatctaaatctgaccgaggaggtggagctcccagaggtggaggaagaggtggaacttctgctggccgtggacgagaaagaaatagatctaacttccgaggagaaagaggtggctttagagggggccgtggaggagcacacagaggtggctttccacctcgataattgttgaagacattgaacctattcatcctcctctaaccttctttattgtaattaaatttcaagtgggagacttaactttagaactcacttccagcttgcactttgctttaatttctctgagctgcaagaatgtcttagcgagccttgcttgcagttgtcacacacactgtctggtttttttcaggataaatgaatgattctgccttttgttatgtgcgtgaacagaatggaacaactcaagtagcttcatcttcagagactgaatttattctgatagacttcagctaattacaaaggattttgctaatttttgggaataaataatggaaaaagatccagtctgtggtatcatgctagtgctgacagggccttgatagaatagagttggaaaagatggtaagcttttgtcagggttttaacattttcttgatgaaacaataaaaagaggtaagcttttttcttctttttttttaagttttaaataaactcagatataatttgaatactgaagaaattaagagactttgaacaaaaactcttcccaaatctaaatttgataggggaggtggagattccaggggtgggtgaaagaagagatagaacttagcaggcagacttaaaaaaaaaaaaaaagtttatcatcataatctcaattttgtggctatgactcctaatcacgcttcctaagaagcaaaggaggacaaatattcatgtgctagatagcactgtggtgtggacttgaacttggattgaccttaaattttatattcctcaaataaaagagaggcagcgacaagatacctcattatcagatgcttggtttatacattttgggactaaaatacttggtgatgaaatgacatacacctttaaacttgttatggagatagtttaatgtaaaaccaactacggaaaaccctcaacttaaggatacagcttggaaattggaactgcaattgccttttattaaaaccatatggtgtgatgtttgtttttaaaattatataagactttatgctgtcacttctcttgctgtactgtaattcatgttttaaatgaatttgataatgaaattatactattatcattcttgatgaatacttttcttatttttatgatttttctaatgaaactttaaacttttgagatttgagagtctgttttctataagtagaattactgttgttacaaaatgaaaaaggactgacctaaaatcagtctcttcttttggtctgtgatggattttaatggccgttctgtgctcatatatacctaagatgagattatattacatccaccaaagactcagtttgaagataaggaatgagtgatagaagaaataaggctgagatccttaaaagcctaattaatttaactcgcttaacccattagtactatctagtacaagacccctttttttttgctgaaattatggtatattttcaacttcactaattacaaattatctagatttagaactctatatgtcagcattgacctgggaatgaagtcaggatagagaaattccacttgcctgtgatgggtccttagaagtatcagctaaggagtgaccctgtcctatacacagggctctctattacgttccataccctgggcctacccaaggtgacattcctgctgtttacatggcataggcacctgtgagatcagtgtcacaatttcatcttagaaagaggtaggtatggctgctttgtcggttgaaagttaaggggagccatgatctaccatatttaggaaaaagttatttaaaaaagagcagatggtggaaaaagaatgtaagacccagaatttatccctttgacaatgaatctggcctttttaatagcaggatggaattgattcactagtttttgctaactttcactttcagtaaaggttgaggtgttgtttttgcaatgactgtgtattcattgaggaaaggtttccaatgaaatttcattactctgaaaaaaaaaaaaaaaaa

SEQ ID NO: 28 - protein, 562 aa, Homo sapiens
MALSKSMHARNRYKDKPPDFAYLASKYPDFKQHVQINLNGRVSLNFKDPEAVRALTCTLLREDFGLSIDIPLERLIPTVPLRLNYIHWVEDLIGHQDSDKSTLRRGIDIGTGASCIYPLLGATLNGWYFLATEVDDMCFNYAKKNVEQNNLSDLIKVVKVPQKTLLMDALKEESEIIYDFCMCNPPFFANQLEAKGVNSRNPRRPPPSSVNTGGITEIMAEGGELEFVKRIIHDSLQLKKRLRWYSCMLGKKCSLAPLKEELRIQGVPKVTYTEFCQGRTMRWALAWSFYDDVTVPSPPSKRRKLEKPRKPITFVVLASVMKELSLKASPLRSETAEGIVVVTTWIEKILTDLKVQHKRVPCGKEEVSLFLTAIENSWIHLRRKKRERVRQLREVPRAPEDVIQALEEKKPTPKESGNSQELARGPQERTPCGPALREGEAAAVEGPCPSQESLSQEENPEPTEDERSEEKGGVEVLESCQGSSNGAQDQEASEQFGSPVAERGKRLPGVAGQYLFKCLINVKKEVDDALVEMHWVEGQNRDLMNQLCTYIRNQIFRLVAVN

SEQ ID NO: 29 - DNA, 5758 bp, Homo sapiens
acgaggctagatggcttcacaagatggcggcgcgctgggagcgtatcatctgcgtttctaggagcttcgctatgcggctgctttaagattctagggttgtacaggcccacgccagacacgacgtctggcaggaacctcggcctcagagatggctctgagtaaatcaatgcatgcaagaaatagatacaaggacaaacctcctgactttgcatatctggcatccaaatatccagattttaagcagcatgttcagataaatctgaatggaagagtgagccttaattttaaagaccccgaagcagtcagagctctgacgtgtactctcctaagggaagattttggactttctattgatattccattggagagactaattcccacagttcccttgagactcaactatattcactgggtagaagatctgatcggtcaccaggattctgacaaaagtactctccgaagaggaattgacataggcacgggggcatcttgcatctaccccttacttggagcaaccttgaatggctggtatttcctcgcaacagaagtggatgatatgtgtttcaactatgcaaagaaaaatgtggaacagaataacttatctgatctcataaaagtggtgaaagtgccacagaagacactcctgatggatgctcttaaagaagaatctgagataatctatgacttttgcatgtgcaaccctcccttttttgccaatcaattggaagccaagggagtaaactcacgaaatcctcgaagacctccgcctagttctgttaatacaggaggcatcacagagatcatggcagaaggaggtgaattagagtttgttaaaaggatcatccatgacagtctacaacttaaaaaaagattaagatggtatagctgcatgctgggaaagaaatgcagcctggcgcctctgaaggaggagcttcgcatacaaggggttcccaaagtaacgtacactgaattctgtcaaggtcggacaatgagatgggccttagcttggagtttttatgatgatgtcacagtaccatcaccaccaagtaagcgaagaaaattagagaaaccgagaaaacccataacattcgtggtgctggcgtccgtgatgaaggaattatccctcaaagcatcacctctgcgctcggagacggcggaaggcatagtcgttgtcacgacatggattgaaaaaattctcactgatttgaaggtccagcataaacgagttccctgtggaaaagaggaagtcagccttttcctaacggccatagaaaactcctggattcatttaaggagaaagaaaagagagcgtgtgagacagctgagagaagttccccgagctcctgaggacgtcattcaggccttggaagagaaaaagcccacccccaaagagtctggcaatagccaagaactggccaggggcccccaggagaggaccccctgtgggcctgctctgcgggaaggcgaggctgccgctgtggagggcccgtgcccgagccaggagtccctgtcccaggaggaaaacccggaacccacggaggatgaaaggagtgaggaaaagggaggggtggaggttttggaaagttgtcaaggctctagcaacggagcccaggaccaagaggcttctgagcagttcggcagcccagtggctgaaagggggaaacgtctcccaggagtggccggacagtacctgtttaagtgtttgataaacgttaagaaggaggtggacgatgccttagtggagatgcactgggttgagggccagaacagggatctgatgaaccagctttgcacctacatacgtaaccaaattttcaggcttgttgcagttaactagaaacctcctgcacagttggaaacgtgttgatagtaacttgctttggagtggcctgtggggtggcaagaggaatcctaccagcggcccattagtagcacgatgtggaattatcttcgaaaacaaaaacctatgaatctgtcccccacctccccccgcctccttcccgctttttgagttacagggagtcgtagtgtggtcatttacaaggaggaattgtggtcatcagtaacaacagaaagccctcagtaaactcccgagggattgcaagctggctcaagctggcccctcagctctggactgcctctgcaaggtcagaagggttgtttgtggagtctgggctgggcagcactgcctagaatatcatgctgtctctgtcacccaagggtgtttcttgaggaggggtggctctctctgcctccagctggaggccctggtaccctgttctaggtcactcttcaagatggggcctaccttgcatcaatcccacaaagggagctgtatggtgggtggtggggaatctgggagagaaaccttagtaatgctgggaaggagcagcagagtctggggaccacccggtaaatggcacattcctgacacctggctgttttgatgttgcttatttcagaagcagaattaggtaagcaaaactccccggtgtgactgaggcacacagaaggcacccatacccccacctccagcctgttgacagtaccattttgtagcagttttactactgtgtgatttttgtttggacatctgaagtagagcttgttttgtttttaaataagaatattcacaaattaaaaaccagcggtcctatttgaatcctggggttagctgagtgagcggctgatgatagaaatgagaaatagaacaaaatagtatgtgccgtaggtagcttaagaaagtctcagatattttgttgctgatcaaatactgtttttttgtggcttcacttgtaatcccccctgtacttacctactcacattggagagttctgaggccggagtaactgtgtccttgaaacacgtttctaattggaatgccagggttcagtagccgtccccccggaaaggggtgaccttttgctgtgcttgatgttgcatcagcagcctagggttctgtttagactaaaatcttggccagagctccttgccatctgctaagaagactggggctgagtagttaagccagccttctgagaggtggctgttggtcaggacgggaagctggtgaccttggcatgtcttggcagcagctagatcaggccctcggcagagacacaggaagcggaactgctgtgccttaacttggctgtggagctggagctggagaaggcagcatactgaccagtggctttttgattgattgtttgttatgaggtggagttttactcttgttgtctaggctggagtgccgtggtgcgatcttagctcactgcaacccccgcctcccgggttcaagcgattctcctgcctcagcctcccaagtagctgggattacaggcacgcgccaccacgcctggctaattttgtgtttttggtagagatgggatttcaccatgttggccaggctaatctcgaactcatgatctcgggtgatccgcccaccttggcctcccaaagtgctgggattacagccgtgagccactactcccagcctctgaccagtgttcttaacctggtccgtggacctccagagagtccatgtacctcctagagttacttctaaaagctctgtgagcatgtgtgtgtgtgtgtgtgtgtgtgtgtattttttttcctggagagagggttcccagaaccctcagacacagacaaaggggtcaataacccactaaggattaagaatcattattctagtccaagcattcatgtgtcaggctgcaaaaaacaatacccagggtcacacagagccaagactcaattcaggaccgtggattcccctggtctagaaattttctgctgtgccagcccacaccaccccactgtccttacctcgagtgaatattacatttgagtcatttgctgggcccaaacctagtttccttggtataattttaggataattgtttaagtggcaactattcattcagtaagtagtaagtacttattgtttgcttgtttcattatgaaagagtggcacatgctcattaaagatttggaaaaatgaaagtcaaaacaacaaaatcaccccgagtcccaaccttctgtaacataaccactcttggcattggcgtgttcctttctagtctctctgtagacggggtgtgtgagtgtgtgggtttaactttggttgtcctcatgctgcgtattcagttttgtattctggtcctttgttcatttaacatcttacaagtatttgtccatgttgtaacagtagtgtattagcttacactccttgcctgttcaaaatgtctttcaggcacagcactggcctttaagcctgtgtcgtagggatttccagagaatgctctgtgtattgaagcacagaaggtgtttctgtgtctcagtgtgtttctgtccctaggtttaaggcttcatgtcatggaggagattttatagatgtcaagctaatgaccttagagttttaaaaaatccgtgaccgtggccaggcgcagtggctcacgcctgtaatcccagcactgtgaggctgagatgggcgcatcgcatgaggtcgggagtttgagaccagcctggccaacatggcgaaaccccgtctctactcaaaatacaaaaattagccgggcatgatagcacgtgcctgtaatcccagctactcgggaggctgaggcaggagactcgcttgaacctgggaggtggaggttgcagtgagccgagaatgccactgcactacagcctgggcgacaaagtgggactgtctcaaaaaaaaaaaaaaaaaaaaaaaaaaaggaacccatgagcaggccagctttcagtctggagccgagtgccttctgtgcatttggatgtttccatttccttccctgagaagattttcttaggctacctagtgagagaacattgaaaatatttttaaaggacatctaagcattgttttggtcatgcatatgctttataattgtgtgttgtttcatagcatatacctctggtacaggtgggcaagtttttctttgaagaaatgggttattgactcatatgtcataaccttgagtgttactctcccggtgtccagaggtcacattcatgttgcggggttggtatgaaattaaatcttggtgatgtgaccctacattctcttctggtccctagaatcggcttctggtctcctgataactgaagtggagacagaagttgagcctgttgcccaggcaaactaaagctgcttttgttcttcggaatctgctttgcctccgtcagcctgcttccttccccacacatgctggccgcactgtccccactccagacctctgctgtgtgtcctgggcagggccgcgttttggcagtaccctttcaactcatcctaagcttcgtgtagattactttagtatatattttttataaaacataaagcctttcctctcgatggaaatcaaagcttaccatgtgagcactcgaacttctaagttgtgacaggaataacaaaactgcaaggagtggaaaagatggaaaagcctgtgggaaatccgaggccttttgaaagaagggagctgatgacttcacgaccagctcctggagcccctcctttctgctgaagccgcggcatttccctccgtggccacacgagggcacccttggcccttttatcaaagcgccttcacttccccgtgggaatggagacaagtctgtccacggtgttttcttgaaatacccagttgctacccagatttgtatttttatgtaaacaaatacattttcacagaaataaaatttgaaaaataaaagtagaaagagaaaaaaa

SEQ ID NO: 30 - protein, 396 aa, Homo sapiens
MTNEEPLPKKVRLSETDFKVMARDELILRWKQYEAYVQALEGKYTDLNSNDVTGLRESEEKLKQQQQESARRENILVMRLATKEQEMQECTTQIQYLKQVQQPSVAQLRSTMVDPAINLFFLKMKGELEQTKDKLEQAQNELSAWKFTPDSQTGKKLMAKCRMLIQENQELGRQLSQGRIAQLEAELALQKKYSEELKSSQDELNDFIIQLDEEVEGMQSTILVLQQQLKETRQQLAQYQQQQSQASAPSTSRTTASEPVEQSEATSKDCSRLTNGPSNGSSSRQRTSGSGFHREGNTTEDDFPSSPGNGNKSSNSSEERTGRGGSGYVNQLSAGYESVDSPTGSENSLTHQSNDTDSSHDPQEEKAVSGKGNRTVGSRHVQNGLDSSVNVQGSVL

SEQ ID NO: 31 - DNA, 2133 bp, Homo sapiens
ggtttcctccctcagcgccattttgtggcagcgagacccacaaataaaggggagcgcaggggttgcggcgggactaggagcgcggcggggccggcggcagagctgtccggctgcgcggtggcccggggggcccgggcggcagggcaagcagcgcggcctcggcctatgcgaccggtggcgccggcgcggcttctgcctggagaggattcaagatgaccaacgaagaacctcttcccaagaaggttcgattgagtgaaacagacttcaaagttatggcaagagatgagttaattctaagatggaaacaatatgaagcatatgtacaagctttggagggcaagtacacagatcttaactctaatgatgtaactggcctaagagagtctgaagaaaaactaaagcaacaacagcaggagtctgcacgcagggaaaacatccttgtaatgcgactagcaaccaaggaacaagagatgcaagagtgtactactcaaatccagtacctcaagcaagtccagcagccgagcgttgcccaactgagatcaacaatggtagacccagcgatcaacttgtttttcctaaaaatgaaaggtgaactggaacagactaaagacaaactggaacaagcccaaaatgaactgagtgcctggaagtttacgcctgatagccaaacagggaaaaagttaatggcgaagtgtcgaatgcttatccaggagaatcaagagcttggaaggcagctgtcccagggacgtattgcacaacttgaagcagagttggctttacagaagaaatacagtgaggagcttaaaagcagtcaggatgaactgaatgacttcatcatccagcttgatgaagaagtagagggtatgcagagtaccattctagttctgcagcagcagctgaaggagacacgccagcagttggctcagtaccagcagcagcagtctcaggcctctgccccaagtaccagcaggactacagcttctgaacctgtagaacagtcagaggccacaagtaaagactgcagtcgtctgacaaacggaccaagtaatggtagctcctcccgccagaggacgtctgggtctggatttcacagggagggcaacacaaccgaagatgactttccttcttctccagggaatggtaataagtcctccaacagctcagaggagagaactggcagaggaggtagtggttacgtaaatcaactcagtgcggggtatgaaagtgtagactctcccacgggcagtgaaaactctctcacacaccaatcaaatgacacagactccagtcatgaccctcaagaggagaaagcagtgagtgggaaaggtaatcgaactgtgggttcccgccacgttcagaatggcttggactcaagtgtaaatgtacagggttcagttttgtaatattttttcagcaaatttttatacagtgtcatttaatttgggagaggatactgtccagaaaattaatgcatacttttgtcacaatttgcctttttgtgggtgtacgttttggtttttttttgttgttttttttctttgttttttttttcttttctttttttttttttttttttttttttgcttcaatacttctgccgctttggaaattgtaacagttaattactttgaatgttgctaaaaggacattttgtgtagggtcaagttatttttatatgagttaatgtgaaattgtaaatggaaatttttccttaaaatacaacacaatgatgtctgtataaatctgtctgtttagaatctgtgctgtgtaagggcattcgtactcatgctgttactgtacttatgcaccattcagacttgttagagtagatgtgggtttatgactgccaagtttgcccagtacagtagttttttatcactaaaagttggactcattgatggagtcctgtagtagtttcagtgttagatacagttttttccaccatacatctgtgcattttctctttaggtgactgtttaagaaatttgtgtgcatagttactcagtttttatgaactgttgtatcctgttaatgcatattgctctgtgactccagtatatcttacctgtactgaccaaacctaaataaagatttttattgtaactccttaaaaaaaaaaaaaaaaaaaaaaaa

SEQ ID NO: 32 - protein, 505 aa, Homo sapiens
MKRTPTAEEREREAKKLRLLEELEDTWLPYLTPKDDEFYQQWQLKYPKLILREASSVSEELHKEVQEAFLTLHKHGCLFRDLVRIQGKDLLTPVSRILIGNPGCTYKYLNTRLFTVPWPVKGSNIKHTEAEIAAACETFLKLNDYLQIETIQALEELAAKEKANEDAVPLCMSADFPRVGMGSSYNGQDEVDIKSRAAYNVTLLNFMDPQKMPYLKEEPYFGMGKMAVSWHHDENLVDRSAVAVYSYSCEGPEEESEDDSHLEGRDPDIWHVGFKISWDIETPGLAIPLHQGDCYFMLDDLNATHQHCVLAGSQPRFSSTHRVAECSTGTLDYILQRCQLALQNVCDDVDNDDVSLKSFEPAVLKQGEEIHNEVEFEWLRQFWFQGNRYRKCTDWWCQPMAQLEALWKKMEGVTNAVLHEVKREGLPVEQRNEILTAILASLTARQNLRREWHARCQSRIARTLPADQKPECRPYWEKDDASMPLPFDLTDIVSELRGQLLEAKP

SEQ ID NO: 33 - DNA, 4313 bp, Homo sapiens
ctacgctcttccagctgtcggacctgggaaattctcctgtgctaaatcccgtggcgctcgcgggtgtcgccgcggtgcatcctgggagttgtagttttttctactcagagggagaatagctccagacgggagcaggacgctgagagaactacatgcaggaggcggggtccagggcgagggatctacgcagcttgcggtggcgaaggcggctttagtggcagcatgaagcgcaccccgactgccgaggaacgagagcgcgaagctaagaaactgaggcttcttgaagagcttgaagacacttggctcccttatctgacccccaaagatgatgaattctatcagcagtggcagctgaaatatcctaaactaattctccgagaagccagcagtgtatctgaggagctccataaagaggttcaagaagcctttctcacactgcacaagcatggctgcttatttcgggacctggttaggatccaaggcaaagatctgctcactccggtatctcgcatcctcattggtaatccaggctgcacctacaagtacctgaacaccaggctctttacggtcccctggccagtgaaagggtctaatataaaacacaccgaggctgaaatagccgctgcttgtgagaccttcctcaagctcaatgactacctgcagatagaaaccatccaggctttggaagaacttgctgccaaagagaaggctaatgaggatgctgtgccattgtgtatgtctgcagatttccccagggttgggatgggttcatcctacaacggacaagatgaagtggacattaagagcagagcagcatacaacgtaactttgctgaatttcatggatcctcagaaaatgccatacctgaaagaggaaccttattttggcatggggaaaatggcagtgagctggcatcatgatgaaaatctggtggacaggtcagcggtggcagtgtacagttatagctgtgaaggccctgaagaggaaagtgaggatgactctcatctcgaaggcagggatcctgatatttggcatgttggttttaagatctcatgggacatagagacacctggtttggcgataccccttcaccaaggagactgctatttcatgcttgatgatctcaatgccacccaccaacactgtgttttggccggttcacaacctcggtttagttccacccaccgagtggcagagtgctcaacaggaaccttggattatattttacaacgctgtcagttggctctgcagaatgtctgtgacgatgtggacaatgatgatgtctctttgaaatcctttgagcctgcagttttgaaacaaggagaagaaattcataatgaggtcgagtttgagtggctgaggcagttttggtttcaaggcaatcgatacagaaagtgcactgactggtggtgtcaacccatggctcaactggaagcactgtggaagaagatggagggtgtgacaaatgctgtgcttcatgaagttaaaagagaggggctccccgtggaacaaaggaatgaaatcttgactgccatccttgcctcgctcactgcacgccagaacctgaggagagaatggcatgccaggtgccagtcacgaattgcccgaacattacctgctgatcagaagccagaatgtcggccatactgggaaaaggatgatgcttcgatgcctctgccgtttgacctcacagacatcgtttcagaactcagaggtcagcttctggaagcaaaaccctagaaggagcacaagtctcaggcggaggagaaaaagagatcggcttttctcctccaacgttgtcatgggcttaagcaagagcagtggagacttctcttggcccctagattgtagcacccgggtcccaatccaaaacagctaggaaatggtgcccatgaagttttaaatgttttaaaatgaccctgtgttatagtctgatttggtgttaaacaggaccttcttcccccaaaattgttcagattataaaatgtgagccattcagcccccaaggtccagggcaggcgacaggaacgagcccagcgtgtgacaaagcctaacctactttcctctttcccaagctttttcagagactctggagtggacccagccctctggggaaagacagaacttagagacatcccagttactcaccacacccatagtgctgtccaatatggtagccactagctagctgtggctacttcaatttaaattcagttttaattttaattaaaaatgcagctcttcagtcgccctggccacatttcaagtgcttaacagcctcatgtggctagtgactgctgtattggacggtacagatatggaacattttcatcatcgaagaaagtcctattggacaacacttctataaaaagtttgagagcaggaattctcatttccattcgtctgtagcttctatccccaaaggcaaagaaactaaaagagaaatgactcattgaagattggcctctttcctttctctaagacaaacctaagtaaaagcctgagctttgagtcctatgctcagcacacgggaaggagatgttaataattaaaataaagttgatatcctgtctttagggagttcccttgatctcttgaaagagacacagccccatttacattatttcgtggatttcaccagcatagtatagtttttttctgtaagtccctcattcttatgtaataacaggtggaactgaggtttgaagaacctcagtggcccatcctgatgacattggagactcaaagagacaagagagagtagggtttaaaacctgagctttaagactcccactagcttcgtgtcctttggcatgttaacgtgcctcagtttcctcatctgtataatggggatatatgaaaggcaccagtcctaaggtgaacattaagtgagatgattctagttacagacttagaacaatttccagcacatagttaaatatccaggaaattctggtactgttatgtgtgggtgagctgacctggatgtagatgttttcctctctcttgctgacccctccgccagttttgtcttgtgatgccattaacacatctctccctttctgacctggctcctgcccattggtgtcccaagaaatcgtgagaatagttagccccccgtctccccagcctgttgctttctcgtgtagttgttcacagtagttgagaagttgaagagcttttgcctattgaaggtgcactgagaataaactctttcctgccaccagaattgcagtggttcacggcctgcactcattcccatgaatgcagttaatagccacagaaatgtcacattaagcaaagcagccagggtctcatcgtgttgagactcgagtctctcagaccttggattcattccctggtgtctttgagcctcagtttcctcattggtaaaagagaagtgaagcagtgtctcacagggtcattacagagattaaatgaaataaatgaaataacatagaccaggagggcgtggtgtttaaaagtcacagatggggcaccctcgggccatccagcccagtgttttctttagcccctatgatgttcattttttgttatatcccattaggtgcccatatttaaaaattgggagatttcacataaaattaaaaggtctgcattttcttttttcttttctttttttttttttttgagacacagtctcactctgtcaccaggctagagtgcagtggcacgatctcagctcactgcaacctctgcctcccaggttcaagtaattctcctgcctcagcctcccaagtagctgggactacaggcacgtgccaccacgcccagctaatttttgtatttttagcagagatggggtttcaccacattggccaggatggtctcgatctcaacctcgtgatccacccacctcggtctcccaaagcgctgggattacaggcgtgagccaccgcgccaagccaaggtctgcatttttctttagaactcagaacacccaatagtcctaggcccccatcctcgcatggcagcaagctaaataagcatcttcccactgcgagttggggcatgacccagcctatggtttgccatactccctctttttctccgttttttcattaattgtgaacctgacctgcatcaccctttcatgtcagtgctctccaaacctgcttgcttgcacccctctagtcgaaatattttgtgcttaccccaatatatgtgtgtgactattgaactctattcgtagactgcttgtactaatgtcatttgcatcataaaatattcatatccaataaacatattaaaaggatgagataagaaaccgaaaaaaaaaaaaaaaaaaaaaa

SEQ ID NO: 34 - protein, 394 aa, Homo sapiens
MAAASGYTDLREKLKSMTSRDNYKAGSREAAAAAAAAVAAAAAAAAAAEPYPVSGAKRKYQEDSDPERSDYEEQQLQKEEEARKVKSGIRQMRLFSQDECAKIEARIDEVVSRAEKGLYNEHTVDRAPLRNKYFFGEGYTYGAQLQKRGPGQERLYPPGDVDEIPEWVHQLVIQKLVEHRVIPEGFVNSAVINDYQPGGCIVSHVDPIHIFERPIVSVSFFSDSALCFGCKFQFKPIRVSEPVLSLPVRRGSVTVLSGYAADEITHCIRPQDIKERRAVIILRKTRLDAPRLETKSLSSSVLPPSYASDRLSGNNRDPALKPKRSHRKADPDAAHRPRILEMDKEENRRSVLLPTHRRRGSFSSENYWRKSYESSEDCSEAAGSPARKVKMRRH

SEQ ID NO: 35 - DNA, 3449 bp, Homo sapiens
cggacgatgccgtgacgcggcacggcgacactgttggcaatatgagcgcacccctgtagagggagcccttcggtcctggaggcggcgcggcgtgaagacaggttgctatttgagagcgttcccttgaagcccctcagagagtgggggaggggcggcggacggcaagcggttcctgtctgcgcttgcgccggcgcctctgccgacccggcctgcacgcacgcgcatgcccgtagcgcgcggagccgcggtggccggcagcactgcgcgtgcgcggtgaggagcccgctaaggagcggcgctggcggacgtcgggctggctgcccgtgacgtcgtgcggagagctttaaagtgcgggccgggccgggcgtccgagggtctggtcgggagtcgggccgcgtctccgcagcagccctccgcggcatgaggcgctgccggcgcccctgccccgcgggacgtggagaaggtggaggaggaagaagccccgttgtcgccaccgttgcatgacccgccgctcctgaggccctaccccacgcccggaccctcgacgccccccgccgggtcccccactcacgcatgggggttcggcgctaaggacccccctccctccgggggccccggggcgcgtccccttagagccatgcccggctgccccgcccgccccggaggaccctagagcagcgtcgtgggggccatggcggccgccagcggctacacggacctgcgtgagaagctcaagtccatgacgtcccgggacaactataaggcgggcagccgggaggccgccgccgctgccgcagccgccgtagccgccgcagccgcagccgccgctgccgccgaaccttaccctgtgtccggggccaagcgcaagtatcaggaggactcggaccccgagcgcagcgactatgaggagcagcagctgcagaaggaggaggaggcgcgcaaggtgaagagcggcatccgccagatgcgcctcttcagccaggacgagtgcgccaagatcgaggcccgcattgacgaggtggtgtcccgcgctgagaagggcctgtacaacgagcacacggtggaccgggccccactgcgcaacaagtacttcttcggcgaaggctacacttacggcgcccagctgcagaagcgcgggcccggccaggagcgcctctacccgccgggcgacgtggacgagatccccgagtgggtgcaccagctggtgatccaaaagctggtggagcaccgcgtcatccccgagggcttcgtcaacagcgccgtcatcaacgactaccagcccggcggctgcatcgtgtctcacgtggaccccatccacatcttcgagcgccccatcgtgtccgtgtccttctttagcgactctgcgctgtgcttcggctgcaagttccagttcaagcctattcgggtgtcggaaccagtgctttccctgccggtgcgcaggggaagcgtgactgtgctcagtggatatgctgctgatgaaatcactcactgcatacggcctcaggacatcaaggagcgccgagcagtcatcatcctcaggaagacaagattagatgcaccccggttggaaacaaagtccctgagcagctccgtgttaccacccagctatgcttcagatcgcctgtcaggaaacaacagggaccctgctctgaaacccaagcggtcccaccgcaaggcagaccctgatgctgcccacaggccacggatcctggagatggacaaggaagagaaccggcgctcggtgctgctgcccacacaccggcggaggggtagcttcagctctgagaactactggcgcaagtcatacgagtcctcagaggactgctctgaggcagcaggcagccctgcccgaaaggtgaagatgcggcggcactgagtctacccgccgccctcctgggaactctggctcatccttacgtagttgcccctccttttgttttgagggttttgtttttgttcattggggggtttttgttttttgttttttgttttttttgattctatatatttttccttggttttgttgcctgttagggctgaagaatagaattggccaggacctaggttctcatattcttggtattcctcctggatggaaaggctgttggcatcaataggggacagaggctgatgctggagtggccagtagaggtggtggagcagagcagccatcttttaagtggggctgtatcaggctgggtttatttaaaagcaacaaaatgttttggttaagaaaattattttgctttcagtgtaaatcttcgcagtgttctaaacaaagttcagtcttctgctcgcccctttccctcactgatgtctgcacttggttgaggtctcctggagcctcacaggctctgctgttctccacttctcacctgccatccacgccctgcaagctcatgcaaacaccctttcttcctcctgcggcagagttgttcaggttgcctgggcaggggcttaaacagtgccagcccctgccatcccaaagctattgttaagccccccaggcgtcctccacccacgcccactagcctgccatgtccacagttccttgggctgctgaggggctagtgcagtggtcctgacctctcttatcaagagcacacttctttgctggttgctccttttgagcatatgcgtgtgattatttggaacagttagacttgccacgttgggtcagttttagaaattgtttctagctagagggactggtgtccttccaagtctagcatttggggtatggaaaattgttgtggtgtgtggtagggtttttgttttcttttttgagttttttttccccctttagtctcctggctttttcctttcccttcccttctccactggccagcttgggcctcatcctcatgtcatccttctaggaaggcgcctgccccatcttgtctgccggcagcatgcatccaaggccagagctcaggcctgcagactgggctggtgcctcctccgcttcagggtatgggagttggtgaaggggctttcaaaaaataataaggaaaaaaaggtaaagtctttggtagcttctatccactcagatcctggaaggcagcaaggttttgtggatctagattcattaggaatgtcttcttgtcagccaggccaggacccgggcttgccaagagcagaggccctcccagcaaccaggataccaccactttgggggctttgtgtacagaggtccgggtctgagacctcataggctgcagaaatctggggcagccaccatcaagaagcccctctcaggggccagaactcctttgccagcgtggatttctcaagtcgggactgcataattaaagcagttgcagttttattttttttacagcttttttcccaaaaatgatttgtagttgtgtgtgcagcacttcgccctgatatgtgtgctctacaataaaaaccaaatctaatatattttgaaaaaaaaaaaaaaaaaa

(19) United States
(12) Patent Application Publication
(10) Pub. No.: US 2021/0332344 A1
(43) Pub. Date: Oct. 28, 2021

(54) DIRECTED MODIFICATION OF RNA

(71) Applicant: The Regents of the University of California, Oakland, CA (US)

(72) Inventors: Eugene Yeo, La Jolla, CA (US); Kristopher Brannan, La Jolla, CA (US)

(21) Appl. No.: 17/272,009

(22) PCT Filed: Aug. 30, 2019
(86) PCT No.: PCT/US2019/049197

§ 371 Date: Feb. 26, 2021

Related U.S. Application Data
(60) Provisional application No. 62/726,145, filed on Aug. 31, 2018.

(51) Int. Cl. C12N 9/78; C12N 9/22; C12N 15/11; C12N 15/90; A61K 38/50; A61K 38/46

(52) U.S. Cl. CPC C12N 9/78; C12N 9/22; C12Y 305/04005; C12N 15/11; C12N 2310/20; A61K 38/50; A61K 38/465; C12N 2800/80; C12N 15/907

(57) ABSTRACT

Described herein are compositions, systems, methods, and kits utilizing CRISPR-Cas protein fusions comprising a guide nucleotide sequence-programmable RNA binding protein and a RNA base modification protein. The compositions, systems, methods, and kits described herein are useful to modulate RNA methylation and/or cytidine deamination.

### CROSS-REFERENCE TO RELATED APPLICATIONS

This application claims priority to: U.S. Patent Application Ser. No. 62/726,145, filed Aug. 31, 2018, which is incorporated hereby reference in its entirety.

### STATEMENT OF GOVERNMENT SUPPORT

This invention was made with government support under HG004659 awarded by the National Institutes of Health. The government has certain rights in the invention.

### BACKGROUND

Present strategies aimed to target and manipulate RNA in living cells mainly rely on the use of antisense oligonucleotides (ASO) or engineered RNA binding proteins (RBP). Although ASO therapies have been shown great promise in eliminating pathogenic transcripts or modulating RBP binding, they are synthetic in construction and thus cannot be encoded within DNA. This complicates potential gene therapy strategies, which would rely on regular administration of ASOs throughout the lifetime of the patient. Furthermore, they are incapable of modulating the genetic sequence of RNA. Although engineered RBPs such as PUF proteins can be designed to recognize target transcripts and fused to RNA modifying effectors to allow for specific recognition and manipulation, these constructs require extensive protein engineering for each target and may prove to be laborious and costly.

Accordingly, there is a need in the art for new methods of modulating RNA that can be simply and rapidly programed for specific mRNA targets. This disclosure satisfies this need and provides related advantages.

### SUMMARY

Described herein is are compositions, systems, methods, and kits to perform RNA modification using CRISPR-Cas protein fusions. These compositions, methods, systems, and kits utilize the RNA targeting abilities of CRISPR-Cas systems, which use a guide RNA to provide a simple and rapidly programmable system for recognizing RNA molecules in cells. CRISPR-Cas systems also have neutral effects on messenger RNA stability, which makes any measured change to protein expression a function of the fused protein effector. The compositions, systems, methods, and kits described herein provide, for example, high utility and versatility when compared to other compositions, methods, systems, and kits for modulating mRNA.

Accordingly, provided herein are fusion proteins comprising, consisting of, or consisting essentially of: (i) a guide nucleotide sequence-programmable RNA binding protein; and (ii) an effector enzyme. In one aspect, described herein are compositions, systems, methods, and kits to modulate RNA methylation using CRISPR-Cas protein fusions. In some embodiments, provided herein are fusion proteins comprising, consisting of, or consisting essentially of: (i) a guide nucleotide sequence-programmable RNA binding protein; and (ii) an RNA methylation modification protein (RMMP), or an equivalent thereof. In another aspect, described herein are compositions, systems, methods, and kits to direct cytidine-to-uridine conversions in target RNA using CRISPR-Cas protein fusions. In some embodiments, provided herein are fusion proteins comprising, consisting of, or consisting essentially of: (i) a guide nucleotide sequence-programmable RNA binding protein; and (ii) an enzyme with cytidine deaminase activity.

In some embodiments, the guide nucleotide sequence-programmable RNA binding protein is selected from: Cas9, modified Cas9, Cas13a, Cas13b, CasRX/Cas13d, and a biological equivalent of each thereof. In some embodiments, the guide nucleotide sequence-programmable RNA binding protein is selected from: Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), Campylobacter jejuni Cas9 (CjeCas9), and Brevibacillus laterosporus Cas9 (BlatCas9). In some embodiments, the guide nucleotide sequence-programmable RNA binding protein is nuclease inactive.

In some embodiments, the fusion peptide further comprises, consists of, or consists essentially of a linker. In some embodiments, the linker is a peptide linker. In some embodiments, the peptide linker comprises, consists of, or consists essentially of an XTEN linker or one or more repeats of the tri-peptide GGS. In some embodiments, the linker is a non-peptide linker. In some embodiments, the non-peptide linker comprises, consists of, or consists essentially of polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the fusion protein comprises the structure NH₂-[effector enzyme]-[linker]-[guide nucleotide sequence-programmable RNA binding protein]-COOH. In some embodiments, the fusion protein comprises the structure NH₂-[guide nucleotide sequence-programmable RNA binding protein]-[linker]-[effector enzyme]-COOH. In some embodiments, the fusion protein comprises, consists of, or consists essentially of the structure NH₂-[RMMP]-[linker]-[guide nucleotide sequence-programmable RNA binding protein]-COOH. In other embodiments, the fusion protein comprises, consists of, or consists essentially of the structure NH₂-[guide nucleotide sequence-programmable RNA binding protein]-[linker]-[RMMP]—COOH. In some embodiments the fusion protein comprises the structure NH₂-[enzyme with cytidine deaminase activity]-[linker]-[guide nucleotide sequence-programmable RNA binding protein]-COOH. In some embodiments, the fusion protein comprises the structure NH₂-[guide nucleotide sequence-programmable RNA binding protein]-[linker]-[enzyme with cytidine deaminase activity]-COOH.

In some embodiments, the guide nucleotide sequence-programmable RNA binding protein is bound to a guide RNA (gRNA), a crisprRNA (crRNA), or a trans-activating crRNA (tracrRNA).

In some embodiments, the RMMP protein is selected from the group of N6-adenosine-methyltransferase 70 kDa subunit (METTL3), Methyltransferase like 14 (METTL14), Methyltransferase like 16 (METTL16), Wilms tumor 1 associated protein (WTAP), AlkB homolog 5, RNA demelthylase (ALKBH5), and Fat mass and obesity-associated protein (FTO), and a biological equivalent of each thereof. In some embodiments, the RMMP protein has an nucleotide sequence comprising all or part of a sequence selected from NM_001080432, NM 019852, NM_020961, NM 024086, NM_001270531, NM 001270532, NM 001270533, NM 004906, NM_152857, NM 152858, NM_017758, and a biological equivalent of each thereof. In some embodiments, the enzyme with cytidine deaminase activity is an Apolipoprotein B mRNA editing enzyme catalytic peptide 1 (APOBEC-1).

In some aspects, provided herein is a polynucleotide encoding a fusion protein comprising, consisting of, or consisting essentially of (i) a guide nucleotide sequence-programmable RNA binding protein; and (ii) an effector enzyme. In some aspects, provided herein is a polynucleotide encoding a fusion protein comprising, consisting of, or consisting essentially of: (i) a guide nucleotide sequence-programmable RNA binding protein; and (ii) a RNA methylation modification protein (RMMP), or an equivalent thereof, or an enzyme with cytidine deaminase activity.

In some aspects, provided herein is a vector comprising, consisting of, or consisting essentially of a polynucleotide encoding a fusion protein comprising, consisting of, or consisting essentially of: (i) a guide nucleotide sequence-programmable RNA binding protein; and (ii) an effector enzyme, optionally wherein the vector is an adenoviral vector, an adeno-associated viral vector, or a lentiviral vector. In some embodiments, the vector further comprises an expression control element. In some embodiments, the vector further comprises, consists of, or consists essentially of a selectable marker. In some embodiments, the vector further comprises a polynucleotide encoding either (i) a gRNA, or (ii) a crRNA and a tracrRNA. In some embodiments, the gRNA or the crRNA comprises, consists of, or consists essentially of a nucleotide sequence complementary to a target RNA. In some aspects, provided herein is a vector comprising, consisting of, or consisting essentially of a polynucleotide encoding a fusion protein comprising, consisting of, or consisting essentially of: (i) a guide nucleotide sequence-programmable RNA binding protein; and (ii) a RNA methylation modification protein (RMMP), or an equivalent thereof, or an enzyme with cytidine deaminase activity, optionally wherein the vector is an adenoviral vector, an adeno-associated viral vector, or a lentiviral vector.

In some aspects, provided herein is a viral particle comprising a vector comprising, consisting of, or consisting essentially of a polynucleotide encoding a fusion protein comprising, consisting of, or consisting essentially of: (i) a guide nucleotide sequence-programmable RNA binding protein; and (ii) an effector enzyme. In some aspects, provided herein is a viral particle comprising a vector comprising, consisting of, or consisting essentially of a polynucleotide encoding a fusion protein comprising, consisting of, or consisting essentially of: (i) a guide nucleotide sequence-programmable RNA binding protein; and (ii) a RNA methylation modification protein (RMMP), or an equivalent thereof, or an enzyme with cytidine deaminase activity.

In some aspects, provided herein is a cell comprising a fusion protein, a polynucleotide, a vector, or a viral particle as described herein. In some embodiments, the cell is a eukaryotic cell. In some embodiments, the cell is a prokaryotic cell. In some embodiments, the cell is a mammalian cell, optionally a bovine, murine, feline, equine, porcine, canine, simian, or human cell.

In some aspects, provided herein is a system for modulating m⁶A RNA methylation of a target RNA, the system comprising: (a) a fusion protein comprising, consisting of, or consisting essentially of: (i) a guide nucleotide sequence-programmable RNA binding protein; and (ii) a RNA methylation modification protein (RMMP), or an equivalent thereof, and (b) a gRNA; or (c) a crRNA and a tracrRNA; wherein the gRNA or the crRNA comprises, consists of, or consists essentially of a sequence complementary to a target RNA. In some embodiments, the system further comprises a PAMmer. In some embodiments, the target RNA does not comprise a PAM sequence or complement thereof.

In some aspects, provided herein is a method for modulating m⁶A RNA methylation of a target RNA, the method comprising, consisting of, or consisting essentially of contacting the target mRNA with a fusion comprising, consisting of, or consisting essentially of: (i) a guide nucleotide sequence-programmable RNA binding protein; and (ii) a RNA methylation modification protein (RMMP), or an equivalent thereof, wherein the guide nucleotide sequence-programmable RNA binding protein binds a gRNA or a crRNA that hybridizes to a region of the target RNA.

In some aspects, provided herein is a method for modulating embryonic stem cell maintenance and/or differentiation, nervous system development, circadian rhythm, heat shock response, meiotic progression, DNA ultraviolet (UV) damage response, or XIST mediated gene silencing, the method comprising, consisting of, or consisting essentially of contacting a target mRNA with a fusion protein comprising, consisting of, or consisting essentially of: (i) a guide nucleotide sequence-programmable RNA binding protein; and (ii) a RNA methylation modification protein (RMMP), or an equivalent thereof, wherein the guide nucleotide sequence-programmable RNA binding protein binds a gRNA or a crRNA that hybridizes to a region of the target RNA. In some embodiments, the target mRNA comprises a PAM sequence or complement thereof. In some embodiments, the target mRNA does not comprise a PAM sequence or complement thereof. In some embodiments, the target mRNA is in a cell. In some embodiments, the cell is a eukaryotic cell. In some embodiments, the cell is a prokaryotic cell. In some embodiments, the eukaryotic cell is a mammalian cell, optionally a bovine, murine, feline, equine, porcine, canine, simian, or human cell. In some embodiments, the cell is in a subject.

In some aspects, provided herein is a method for treating a disease or condition associated with m⁶A RNA methylation of a target RNA in a subject in need thereof, the method comprising, consisting of, or consisting essentially of administering a fusion protein, polynucleotide, vector, viral particle, and/or cell as described herein to the subject, thereby treating the disease or condition associated with m⁶A RNA methylation. In some embodiments, the disease or condition associated with m⁶A RNA methylation is selected from the group consisting of cancer, growth retardation, developmental delay, facial dysmorphism, Alzheimer's disease, diabetes, and major depressive disorder. In some embodiments, the subject is a human. In some embodiments, the methods further comprise, consist of, or consist essentially of administering to the subject: (i) a gRNA complementary to the target RNA, or (ii) a crRNA complementary to the target RNA and a tracrRNA. In some embodiments, the methods further comprise, consist of, or consist essentially of administering a PAMmer to the subject.

In some aspects, provided herein is a method for editing a cytidine base into a uridine base in a target RNA, the method comprising contacting the target RNA with any of the fusion protein described herein, wherein the guide nucleotide sequence-programmable RNA binding protein binds a gRNA or a crRNA that hybridizes to a region of the target RNA.

In some aspects, provided herein is a kit comprising, consisting of, or consisting essentially of one or more of: a fusion protein, polynucleotide, vector, viral particle, and/or cell as described herein; and optionally instructions for use. In some embodiments, the kit further comprises, consists of, or consists essentially of one or more nucleic acids selected from: (i) a gRNA; (ii) a crRNA and a tracrRNA; (iii) a PAMmer; and (iv) a vector for expressing the nucleic acid of (i), (ii), and/or (iii).

In some aspects, provided herein is a non-human transgenic animal comprising, consisting of, or consisting essentially of a fusion protein or viral vector as described herein.

### BRIEF DESCRIPTION OF DRAWINGS

FIG. 1A shows an exemplary design of the Target RNA C-to-U Editing (TRACE) system.

FIG. 1B shows exemplary TRACE effector fusion constructs

FIG. 1C shows exemplary applications of TRACE in living cells FIG. 2A is eCLIP of the RBFOX2-APOBEC1 fusion protein showing binding to the GCAUG binding motif.

FIG. 2B shows enrichment of C-to-U edits at or near RBFOX2 eCLIP binding motifs catalyzed by the RBFOX2-APOBEC1 fusion protein.

FIG. 2C shows binding of the RBFOX2-APOBEC fusion to target RNA DDIT4 and binding-site proximal, specific C-to-U editing.

FIG. 2D shows RBFOX2-APOBEC fusion protein specifically editing the majority of eCLIP target RNAs.

FIG. 2E shows RBFOX2-APOBEC fusion protein specifically enriching for C-to-U edits on RBFOX2 target RNAs.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

### Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 or 12, sequentially numbered, are disclosed in the prior art. Non-limiting exemplary serotypes useful in the methods disclosed herein include any of the 11 or 12 serotypes, e.g., AAV2, AAV5, and AAV8, or variant serotypes, e.g. AAV-DJ. The AAV structural particle is composed of 60 protein molecules made up of VP1, VP2 and VP3. Each particle contains approximately 5 VP1 proteins, 5 VP2 proteins and 50 VP3 proteins ordered into an icosahedral structure.

Also as used herein, “and/or” refers to and encompasses any and all possible combinations of one or more of the associated listed items, as well as the lack of combinations when interpreted in the alternative (“or”).

The term “guide nucleotide sequence-programmable RNA binding protein” refers to a CRISPR-associated, RNA-guided endonuclease such as Streptococcus pyogenes Cas9 (spCas9) and orthologs and biological equivalents thereof. Biological equivalents of Cas9 include but are not limited to Type VI CRISPR systems, such as Cas13a, C2c2, and Cas13b, which target RNA rather than DNA. A guide nucleotide sequence-programmable RNA binding protein may refer to an endonuclease that causes breaks or nicks in RNA as well as other variations such as dead Cas9 or dCas9, which lack endonuclease activity. A guide nucleotide sequence-programmable RNA binding protein may also refer to a “split” protein in which the protein is split into two halves (e.g., C-Cas9 and N-Cas9) and fused with two intein moieties. See, e.g., U.S. Pat. No. 9,074,199 B1; Zetsche et al. (2015) Nat Biotechnol. 33(2):139-42; Wright et al. (2015) PNAS 112(10) 2984-89.

In particular embodiments, the guide nucleotide sequence-programmable RNA binding protein is modified to eliminate endonuclease activity (“nuclease dead”). For example, both RuvC and HNH nuclease domains can be rendered inactive by point mutations (e.g., D10A and H840A in SpCas9), resulting in a nuclease dead Cas9 (dCas9) molecule that cannot cleave target DNA. The dCas9 molecule retains the ability to bind to target RNA based on the gRNA targeting sequence.

Further nonlimiting examples of orthologs and biological equivalents Cas9 are provided in the table below:

| Name | Protein Sequence |
|---|---|
| S. pyogenes Cas9 | MDKKYSIGLDIGTNSVGWAVITDEYKVPSKKFKVLGNTDRHSIK |
| | KNLIGALLFDSGETAEATRLKRTARRRYTRRKNRICYLQEIFSNE |
| | MAKVDDSFFHRLEESFLVEEDKKHERHPIFGNIVDEVAYHEKYPT |
| | IYHLRKKLVDSTDKADLRLIYLALAHMIKFRGHFLIEGDLNPDNS |
| | DVDKLFIQLVQTYNQLFEENPINASGVDAKAILSARLSKSRRLENL |
| | IAQLPGEKKNGLFGNLIALSLGLTPNFKSNFDLAEDAKLQLSKDT |
| | YDDDLDNLLAQIGDQYADLFLAAKNLSDAILLSDILRVNTEITKA |
| | PLSASMIKRYDEHHQDLTLLKALVRQQLPEKYKEIFFDQSKNGYA |
| | GYIDGGASQEEFYKFIKPILEKMDGTEELLVKLNREDLLRKQRTF |
| | DNGSIPHQIHLGELHAILRRQEDFYPFLKDNREKIEKILTFRIPYYV |
| | GPLARGNSRFAWMTRKSEETITPWNFEEVVDKGASAQSFIERMT |
| | NFDKNLPNEKVLPKHSLLYEYFTVYNELTKVKYVTEGMRKPAFL |
| | SGEQKKAIVDLLFKTNRKVTVKQLKEDYFKKIECFDSVEISGVED |
| | RFNASLGTYHDLLKIIKDKDFLDNEENEDILEDIVLTLTLFEDREMI |
| | EERLKTYAHLFDDKVMKQLKRRRYTGWGRLSRKLINGIRDKQSG |
| | KTILDFLKSDGFANRNFMQLIHDDSLTFKEDIQKAQVSGQGDSLH |
| | EHIANLAGSPAIKKGILQTVKVVDELVKVMGRHKPENIVIEMARE |
| | NQTTQKGQKNSRERMKRIEEGIKELGSQILKEHPVENTQLQNEKL |
| | YLYYLQNGRDMYVDQELDINRLSDYDVDHIVPQSFLKDDSIDNK |
| | VLTRSDKNRGKSDNVPSEEVVKKMKNYWRQLLNAKLITQRKFD |
| | NLTKAERGGLSELDKAGFIKRQLVETRQITKHVAQILDSRMNTKY |
| | DENDKLIREVKVITLKSKLVSDFRKDFQFYKVREINNYHHAHDA |
| | YLNAVVGTALIKKYPKLESEFVYGDYKVYDVRKMIAKSEQEIGK |
| | ATAKYFFYSNIMNFFKTEITLANGEIRKRPLIETNGETGEIVWDKG |
| | RDFATVRKVLSMPQVNIVKKTEVQTGGFSKESILPKRNSDKLIAR |
| | KKDWDPKKYGGFDSPTVAYSVLVVAKVEKGKSKKLKSVKELLG |
| | ITIMERSSFEKNPIDFLEAKGYKEVKKDLIIKLPKYSLFELENGRKR |
| | MLASAGELQKGNELALPSKYVNFLYLASHYEKLKGSPEDNEQKQ |
| | LFVEQHKHYLDEIIEQISEFSKRVILADANLDKVLSAYNKHRDKPI |
| | REQAENIIHLFTLTNLGAPAAFKYFDTTIDRKRYTSTKEVLDATLI |
| | HQSITGLYETRIDLSQLGGD* |
| Staphylococcus | MKRNYILGLDIGITSVGYGIIDYETRDVIDAGVRLFKEANVENNE |
| aureus Cas9 | GRRSKRGARRLKRRRRHRIQRVKKLLFDYNLLTDHSELSGINPYE |
| | ARVKGLSQKLSEEEFSAALLHLAKRRGVHNVNEVEEDTGNELST |
| | KEQISRNSKALEEKYVAELQLERLKKDGEVRGSINRFKTSDYVKE |
| | AKQLLKVQKAYHQLDQSFIDTYIDLLETRRTYYEGPGEGSPFGW |
| | KDIKEWYEMLMGHCTYFPEELRSVKYAYNADLYNALNDLNNLV |
| | ITRDENEKLEYYEKFQIIENVFKQKKKPTLKQIAKEILVNEEDIKG |
| | YRVTSTGKPEFTNLKVYHDIKDITARKEIIENAELLDQIAKILTIYQ |
| | SSEDIQEELTNLNSELTQEEIEQISNLKGYTGTHNLSLKAINLILDE |
| | LWHTNDNQIAIFNRLKLVPKKVDLSQQKEIPTTLVDDFILSPVVKR |
| | SFIQSIKVINAIIKKYGLPNDIIIELAREKNSKDAQKMINEMQKRNR |
| | QTNERIEEIIRTTGKENAKYLIEKIKLHDMQEGKCLYSLEAIPLEDL |
| | LNNPFNYEVDHIIPRSVSFDNSFNNKVLVKQEENSKKGNRTPFQY |
| | LSSSDSKISYETFKKHILNLAKGKGRISKTKKEYLLEERDINRFSVQ |
| | KDFINRNLVDTRYATRGLMNLLRSYFRVNNLDVKVKSINGGFTS |
| | FLRRKWKFKKERNKGYKHHAEDALIIANADFIFKEWKKLDKAK |
| | KVMENQMFEEKQAESMPEIETEQEYKEIFITPHQIKHIKDFKDYK |
| | YSHRVDKKPNRELINDTLYSTRKDDKGNTLIVNNLNGLYDKDND |
| | KLKKLINKSPEKLLMYHHDPQTYQKLKLIMEQYGDEKNPLYKYY |
| | EETGNYLTKYSKKDNGPVIKKIKYYGNKLNAHLDITDDYPNSRN |
| | KVVKLSLKPYRFDVYLDNGVYKFVTVKNLDVIKKENYYEVNSK |
| | CYEEAKKLKKISNQAEFIASFYNNDLIKINGELYRVIGVNNDLLNR |
| | IEVNMIDITYREYLENMNDKRPPRIIKTIASKTQSIKKYSTDILGNL |
| | YEVKSKKHPQIIKKG* |
| S. thermophilus | MSDLVLGLDIGIGSVGVGILNKVTGEIIHKNSRIFPAAQAENNLVR |
| CRISPR 1 Cas9 | RTNRQGRRLARRKKHRRVRLNRLFEESGLITDFTKISINLNPYQLR |
| | VKGLTDELSNEELFIALKNMVKHRGISYLDDASDDGNSSVGDYA |
| | QIVKENSKQLETKTPGQIQLERYQTYGQLRGDFTVEKDGKKHRLI |
| | NVFPTSAYRSEALRILQTQQEFNPQITDEFINRYLEILTGKRKYYH |
| | GPGNEKSRTDYGRYRTSGETLDNIFGILIGKCTFYPDEFRAAKASY |
| | TAQEFNLLNDLNNLTVPTETKKLSKEQKNQIINYVKNEKAMGPA |
| | KLFKYIAKLLSCDVADIKGYRIDKSGKAEIHTFEAYRKMKTLETL |
| | DIEQMDRETLDKLAYVLTLNTEREGIQEALEHEFADGSFSQKQVD |
| | ELVQFRKANSSIFGKGWHNFSVKLMMELIPELYETSEEQMTILTR |
| | LGKQKTTSSSNKTKYIDEKLLTEEIYNPVVAKSVRQAIKIVNAAIK |
| | EYGDFDNIVIEMARETNEDDEKKAIQKIQKANKDEKDAAMLK |
| | AANQYNGKAELPHSVFHGHKQLATKIRLWHQQGERCLYTGKTIS |
| | IHDLINNSNQFEVDHILPLSITFDDSLANKVLVYATANQEKGQRTP |
| | YQALDSMDDAWSFRELKAFVRESKTLSNKKKEYLLTEEDISKFD |
| | VRKKFIERNLVDTRYASRVVLNALQEHFRAHKIDTKVSVVRGQF |
| | TSQLRRHWGIEKTRDTYHHHAVDALIIAASSQLNLWKKQKNTLV |
| | SYSEDQLLDIETGELISDDEYKESVFKAPYQHFVDTLKSKEFEDSI |
| | LFSYQVDSKFNRKISDATIYATRQAKVGKDKADETYVLGKIKDIY |
| | TQDGYDAFMKIYKKDKSKFLMYRHDPQTFEKVIEPILENYPNKQI |
| | NDKGKEVPCNPFLKYKEEHGYIRKYSKKGNGPEIKSLKYYDSKL |
| | GNHIDITPKDSNNKVVLQSVSPWRADVYFNKTTGKYEILGLKYA |
| | DLQFDKGTGTYKISQEKYNDIKKKEGVDSDSEFKFTLYKNDLLLV |
| | KDTETKEQQLFRFLSRTMPKQKHYVELKPYDKQKFEGGEALIKV |
| | LGNVANSGQCKKGLGKSNISIYKVRTDVLGNQHIIKNEGDKPKLD |
| | F* |
| N. meningitidis Cas9 | MAAFKPNPINYILGLDIGIASVGWAMVEIDEDENPICLIDLGVRVF |
| | ERAEVPKTGDSLAMARRLARSVRRLTRRRAHRLLRARRLLKREG |
| | VLQAADFDENGLIKSLPNTPWQLRAAALDRKLTPLEWSAVLLHLI |
| | KHRGYLSQRKNEGETADKELGALLKGVADNAHALQTGDFRTPA |
| | ELALNKFEKESGHIRNQRGDYSHTFSRKDLQAELILLFEKQKEFG |
| | NPHVSGGLKEGIETLLMTQRPALSGDAVQKMLGHCTFEPAEPKA |
| | AKNTYTAERFIWLTKLNNLRILEQGSERPLTDTERATLMDEPYRK |
| | SKLTYAQARKLLGLEDTAFFKGLRYGKDNAEASTLMEMKAYHA |
| | ISRALEKEGLKDKKSPLNLSPELQDEIGTAFSLFKTDEDITGRLKD |
| | RIQPEILEALLKHISFDKFVQISLKALRRIVPLMEQGKRYDEACAEI |
| | YGDHYGKKNTEEKIYLPPIPADEIRNPVVLRALSQARKVINGVVR |
| | RYGSPARIHIETAREVGKSFKDRKEIEKRQEENRKDREKAAAKFR |
| | EYFPNFVGEPKSKDILKLRLYEQQHGKCLYSGKEINLGRLNEKGY |
| | VEIDHALPFSRTWDDSFNNKVLVLGSENQNKGNQTPYEYFNGKD |
| | NSREWQEFKARVETSRFPRSKKQRILLQKFDEDGFKERNLNDTRY |
| | VNRFLCQFVADRMRLTGKGKKRVFASNGQITNLLRGFWGLRKV |
| | RAENDRHHALDAVVVACSTVAMQQKITRFVRYKEMNAFDGKTI |
| | DKETGEVLHQKTHFPQPWEFFAQEVMIRVFGKPDGKPEFEEADT |
| | PEKLRTLLAEKLSSRPEAVHEYVTPLFVSRAPNRKMSGQGHMET |
| | VKSAKRLDEGVSVLRVPLTQLKLKDLEKMVNREREPKLYEALKA |
| | RLEAHKDDPAKAFAEPFYKYDKAGNRTQQVKAVRVEQVQKTG |
| | VWVRNHNGIADNATMVRVDVFEKGDKYYLVPIYSWQVAKGILP |
| | DRAVVQGKDEEDWQLIDDSFNFKFSLHPNDLVEVITKKARMFGY |
| | FASCHRGTGNINIRIHDLDHKIGKNGILEGIGVKTALSFQKYQIDEL |
| | GKEIRPCRLKKRPPVR* |
| Parvibaculum | MERIFGFDIGTTSIGFSVIDYSSTQSAGNIQRLGVRIFPEARDPDGTP |
| lavamentivorans | LNQQRRQKRMMRRQLRRRRIRRKALNETLHEAGFLPAYGSADW |
| Cas9 | PVVMADEPYELRRRGLEEGLSAYEFGRAIYHLAQHRHFKGRELE |
| | ESDTPDPDVDDEKEAANERAATLKALKNEQTTLGAWLARRPPSD |
| | RKRGIHAHRNVVAEEFERLWEVQSKFHPALKSEEMRARISDTIFA |
| | QRPVFWRKNTLGECRFMPGEPLCPKGSWLSQQRRMLEKLNNLAI |
| | AGGNARPLDAEERDAILSKLQQQASMSWPGVRSALKALYKQRG |
| | EPGAEKSLKFNLELGGESKLLGNALEAKLADMFGPDWPAHPRKQ |
| | EIRHAVHERLWAADYGETPDKKRVIILSEKDRKAHREAAANSFV |
| | ADFGITGEQAAQLQALKLPTGWEPYSIPALNLFLAELEKGERFGA |
| | LVNGPDWEGWRRTNFPHRNQPTGEILDKLPSPASKEERERISQLR |
| | NPTVVRTQNELRKVVNNLIGLYGKPDRIRIEVGRDVGKSKREREE |
| | IQSGIRRNEKQRKKATEDLIKNGIANPSRDDVEKWILWKEGQERC |
| | PYTGDQIGFNALFREGRYEVEHIWPRSRSFDNSPRNKTLCRKDVN |
| | IEKGNRMPFEAFGHDEDRWSAIQIRLQGMVSAKGGTGMSPGKVK |
| | RFLAKTMPEDFAARQLNDTRYAAKQILAQLKRLWPDMGPEAPV |
| | KVEAVTGQVTAQLRKLWTLNNILADDGEKTRADHRHHAIDALT |
| | VACTHPGMTNKLSRYWQLRDDPRAEKPALTPPWDTIRADAEKA |
| | VSEIVVSHRVRKKVSGPLHKETTYGDTGTDIKTKSGTYRQFVTRK |
| | KIESLSKGELDEIRDPRIKEIVAAHVAGRGGDPKKAFPPYPCVSPG |
| | GPEIRKVRLTSKQQLNLMAQTGNGYADLGSNHHIAIYRLPDGKA |
| | DFEIVSLFDASRRLAQRNPIVQRTRADGASFVMSLAAGEAIMIPEG |
| | SKKGIWIVQGVWASGQVVLERDTDADHSTTTRPMPNPILKDDAK |
| | KVSIDPIGRVRPSND* |
| Corynebacter | MKYHVGIDVGTFSVGLAAIEVDDAGMPIKTLSLVSHIHDSGLDPD |
| diphtheria Cas9 | EIKSAVTRLASSGIARRTRRLYRRKRRRLQQLDKFIQRQGWPVIEL |
| | EDYSDPLYPWKVRAELAASYIADEKERGEKLSVALRHIARHRGW |
| | RNPYAKVSSLYLPDGPSDAFKAIREEIKRASGQPVPETATVGQMV |
| | TLCELGTLKLRGEGGVLSARLQQSDYAREIQEICRMQEIGQELYR |
| | KIIDVVFAAESPKGSASSRVGKDPLQPGKNRALKASDAFQRYRIA |
| | ALIGNLRVRVDGEKRILSVEEKNLVFDHLVNLTPKKEPEWVTIAEI |
| | LGIDRGQLIGTATMTDDGERAGARPPTHDTNRSIVNSRIAPLVDW |
| | WKTASALEQHAMVKALSNAEVDDFDSPEGAKVQAFFADLDDDV |
| | HAKLDSLHLPVGRAAYSEDTLVRLTRRMLSDGVDLYTARLQEFG |
| | IEPSWTPPTPRIGEPVGNPAVDRVLKTVSRWLESATKTWGAPERV |
| | IIEHVREGFVTEKRAREMDGDMRRRAARNAKLFQEMQEKLNVQ |
| | GKPSRADLWRYQSVQRQNCQCAYCGSPITFSNSEMDHIVPRAGQ |
| | GSTNTRENLVAVCHRCNQSKGNTPFAIWAKNTSIEGVSVKEAVE |
| | RTRHWVTDTGMRSTDFKKFTKAVVERFQRATMDEEIDARSMES |
| | VAWMANELRSRVAQHFASHGTTVRVYRGSLTAEARRASGISGK |
| | LKFFDGVGKSRLDRRHHAIDAAVIAFTSDYVAETLAVRSNLKQS |
| | QAHRQEAPQWREFTGKDAEHRAAWRVWCQKMEKLSALLTEDL |
| | RDDRVVVMSNVRLRLGNGSAHKETIGKLSKVKLSSQLSVSDIDK |
| | ASSEALWCALTREPGFDPKEGLPANPERHIRVNGTHVYAGDNIGL |
| | FPVSAGSIALRGGYAELGSSFHHARVYKITSGKKPAFAMLRVYTI |
| | DLLPYRNQDLFSVELKPQTMSMRQAEKKLRDALATGNAEYLGW |
| | LVVDDELVVDTSKIATDQVKAVEAELGTIRRWRVDGFFSPSKLRL |
| | RPLQMSKEGIKKESAPELSKIIDRPGWLPAVNKLFSDGNVTVVRR |
| | DSLGRVRLESTAHLPVTWKVQ* |
| Streptococcus | MTNGKILGLDIGIASVGVGIIEAKTGKVVHANSRLFSAANAENNA |
| pasteurtanus Cas9 | ERRGFRGSRRLNRRKKHRVKRVRDLFEKYGIVTDFRNLNLNPYE |
| | LRVKGLTEQLKNEELFAALRTISKRRGISYLDDAEDDSTGSTDYA |
| | KSIDENRRLLKNKTPGQIQLERLEKYGQLRGNFTVYDENGEAHRL |
| | INVFSTSDYEKEARKILETQADYNKKITAEFIDDYVEILTQKRKYY |
| | HGPGNEKSRTDYGRFRTDGTTLENIFGILIGKCNFYPDEYRASKAS |
| | YTAQEYNFLNDLNNLKVSTETGKLSTEQKESLVEFAKNTATLGP |
| | AKLLKEIAKILDCKVDEIKGYREDDKGKPDLHTFEPYRKLKFNLE |
| | SINIDDLSREVIDKLADILTLNTEREGIEDAIKRNLPNQFTEEQISEII |
| | KVRKSQSTAFNKGWHSFSAKLMNELIPELYATSDEQMTILTRLEK |
| | FKVNKKSSKNTKTIDEKEVTDEIYNPVVAKSVRQTIKIINAAVKK |
| | YGDFDKIVIEMPRDKNADDEKKFIDKRNKENKKEKDDALKRAA |
| | YLYNSSDKLPDEVFHGNKQLETKIRLWYQQGERCLYSGKPISIQE |
| | LVHNSNNFEIDHILPLSLSFDDSLANKVLVYAWTNQEKGQKTPYQ |
| | VIDSMDAAWSFREMKDYVLKQKGLGKKKRDYLLTTENIDKIEV |
| | KKKFIERNLVDTRYASRVVLNSLQSALRELGKDTKVSVVRGQFT |
| | SQLRRKWKIDKSRETYHHHAVDALIIAASSQLKLWEKQDNPMFV |
| | DYGKNQVVDKQTGEILSVSDDEYKELVFQPPYQGFVNTISSKGFE |
| | DEILFSYQVDSKYNRKVSDATIYSTRKAKIGKDKKEETYVLGKIK |
| | DIYSQNGFDTFIKKYNKDKTQFLMYQKDSLTWENVIEVILRDYPT |
| | TKKSEDGKNDVKCNPFEEYRRENGLICKYSKKGKGTPIKSLKYY |
| | DKKLGNCIDITPEESRNKVILQSINPWRADVYFNPETLKYELMGL |
| | KYSDLSFEKGTGNYHISQEKYDAIKEKEGIGKKSEFKFTLYRNDLI |
| | LIKDIASGEQEIYRFLSRTMPNVNHYVELKPYDKEKFDNVQELVE |
| | ALGEADKVGRCIKGLNKPNISIYKVRTDVLGNKYFVKKKGDKPK |
| | LDFKNNKK* |
| Neisseria cinerea | MAAFKPNPMNYILGLDIGIASVGWAIVEIDEEENPIRLIDLGVRVF |
| Cas9 | ERAEVPKTGDSLAAARRLARSVRRLTRRRAHRLLRARRLLKREG |
| | VLQAADFDENGLIKSLPNTPWQLRAAALDRKLTPLEWSAVLLHLI |
| | KHRGYLSQRKNEGETADKELGALLKGVADNTHALQTGDFRTPA |
| | ELALNKFEKESGHIRNQRGDYSHTFNRKDLQAELNLLFEKQKEFG |
| | NPHVSDGLKEGIETLLMTQRPALSGDAVQKMLGHCTFEPTEPKA |
| | AKNTYTAERFVWLTKLNNLRILEQGSERPLTDTERATLMDEPYR |
| | KSKLTYAQARKLLDLDDTAFFKGLRYGKDNAEASTLMEMKAYH |
| | AISRALEKEGLKDKKSPLNLSPELQDEIGTAFSLFKTDEDITGRLK |
| | DRVQPEILEALLKHISFDKFVQISLKALRRIVPLMEQGNRYDEACT |
| | EIYGDHYGKKNTEEKIYLPPIPADEIRNPVVLRALSQARKVINGVV |
| | RRYGSPARIHIETAREVGKSFKDRKEIEKRQEENRKDREKSAAKF |
| | REYFPNFVGEPKSKDILKLRLYEQQHGKCLYSGKEINLGRLNEKG |
| | YVEIDHALPFSRTWDDSFNNKVLALGSENQNKGNQTPYEYFNGK |
| | DNSREWQEFKARVETSRFPRSKKQRILLQKFDEDGFKERNLNDTR |
| | YINRFLCQFVADHMLLTGKGKRRVFASNGQITNLLRGFWGLRKV |
| | RAENDRHHALDAVVVACSTIAMQQKITRFVRYKEMNAFDGKTID |
| | KETGEVLHQKAHFPQPWEFFAQEVMIRVFGKPDGKPEFEEADTP |
| | EKLRTLLAEKLSSRPEAVHKYVTPLFISRAPNRKMSGQGHMETV |
| | KSAKRLDEGISVLRVPLTQLKLKDLEKMVNREREPKLYEALKAR |
| | LEAHKDDPAKAFAEPFYKYDKAGNRTQQVKAVRVEQVQKTGV |
| | WVHNHNGIADNATIVRVDVFEKGGKYYLVPIYSWQVAKGILPDR |
| | AVVQGKDEEDWTVMDDSFEFKFVLYANDLIKLTAKKNEFLGYF |
| | VSLNRATGAIDIRTHDTDSTKGKNGIFQSVGVKTALSFQKYQIDE |
| | LGKEIRPCRLKKRPPVR* |
| Campylobacter lari | MRILGFDIGINSIGWAFVENDELKDCGVRIFTKAENPKNKESLALP |
| Cas9 | RRNARSSRRRLKRRKARLIAIKRILAKELKLNYKDYVAADGELPK |
| | AYEGSLASVYELRYKALTQNLETKDLARVILHIAKHRGYMNKNE |
| | KKSNDAKKGKILSALKNNALKLENYQSVGEYFYKEFFQKYKKNT |
| | KNFIKIRNTKDNYNNCVLSSDLEKELKLILEKQKEFGYNYSEDFIN |
| | EILKVAFFQRPLKDFSHLVGACTFFEEEKRACKNSYSAWEFVALT |
| | KIINEIKSLEKISGEIVPTQTINEVLNLILDKGSITYKKFRSCINLHESI |
| | SFKSLKYDKENAENAKLIDFRKLVEFKKALGVHSLSRQELDQIST |
| | HITLIKDNVKLKTVLEKYNLSNEQINNLLEIEFNDYINLSFKALGM |
| | ILPLMREGKRYDEACEIANLKPKTVDEKKDFLPAFCDSIFAHELSN |
| | PVVNRAISEYRKVLNALLKKYGKVHKIHLELARDVGLSKKAREK |
| | IEKEQKENQAVNAWALKECENIGLKASAKNILKLKLWKEQKEICI |
| | YSGNKISIEHLKDEKALEVDHIYPYSRSFDDSFINKVLVFTKENQE |
| | KLNKTPFEAFGKNIEKWSKIQTLAQNLPYKKKNKILDENFKDKQ |
| | QEDFISRNLNDTRYIATLIAKYTKEYLNFLLLSENENANLKSGEKG |
| | SKIHVQTISGMLTSVLRHTWGFDKKDRNNHLHHALDAIIVAYSTN |
| | SIIKAFSDFRKNQELLKARFYAKELTSDNYKHQVKFFEPFKSFREK |
| | ILSKIDEIFVSKPPRKRARRALHKDTFHSENKIIDKCSYNSKEGLQI |
| | ALSCGRVRKIGTKYVENDTIVRVDIFKKQNKFYAIPIYAMDFALGI |
| | LPNKIVITGKDKNNNPKQWQTIDESYEFCFSLYKNDLILLQKKNM |
| | QEPEFAYYNDFSISTSSICVEKHDNKFENLTSNQKLLFSNAKEGSV |
| | KVESLGIQNLKVFEKYIITPLGDKIKADFQPRENISLKTSKKYGLR* |
| T. denticola Cas9 | MKKEIKDYFLGLDVGTGSVGWAVTDTDYKLLKANRKDLWGMR |
| | CFETAETAEVRRLHRGARRRIERRKKRIKLLQELFSQEIAKTDEGF |
| | FQRMKESPFYAEDKTILQENTLFNDKDFADKTYHKAYPTINHLIK |
| | AWIENKVKPDPRLLYLACHNIIKKRGHFLFEGDFDSENQFDTSIQA |
| | LFEYLREDMEVDIDADSQKVKEILKDSSLKNSEKQSRLNKILGLK |
| | PSDKQKKAITNLISGNKINFADLYDNPDLKDAEKNSISFSKDDFDA |
| | LSDDLASILGDSFELLLKAKAVYNCSVLSKVIGDEQYLSFAKVKI |
| | YEKHKTDLTKLKNVIKKHFPKDYKKVFGYNKNEKNNNYSGYV |
| | GVCKTKSKKLIINNSVNQEDFYKFLKTILSAKSEIKEVNDILTEIET |
| | GTFLPKQISKSNAEIPYQLRKMELEKILSNAEKHFSFLKQKDEKGL |
| | SHSEKIIMLLTFKIPYYIGPINDNHKKFFPDRCWVVKKEKSPSGKT |
| | TPWNFFDHIDKEKTAEAFITSRTNFCTYLVGESVLPKSSLLYSEYT |
| | VLNEINNLQIIIDGKNICDIKLKQKIYEDLFKKYKKITQKQISTFIKH |
| | EGICNKTDEVIILGIDKECTSSLKSYIELKNIFGKQVDEISTKNMLE |
| | EIIRWATIYDEGEGKTILKTKIKAEYGKYCSDEQIKKILNLKFSGW |
| | GRLSRKFLETVTSEMPGFSEPVNIITAMRETQNNLMELLSSEFTFT |
| | ENIKKINSGFEDAEKQFSYDGLVKPLFLSPSVKKMLWQTLKLVKE |
| | ISHITQAPPKKIFIEMAKGAELEPARTKTRLKILQDLYNNCKNDAD |
| | AFSSEIKDLSGKIENEDNLRLRSDKLYLYYTQLGKCMYCGKPIEIG |
| | HVFDTSNYDIDHIYPQSKIKDDSISNRVLVCSSCNKNKEDKYPLKS |
| | EIQSKQRGFWNFLQRNNFISLEKLNRLTRATPISDDETAKFIARQL |
| | VETRQATKVAAKVLEKMFPETKIVYSKAETVSMFRNKFDIVKCR |
| | EINDFHHAHDAYLNIVVGNVYNTKFTNNPWNFIKEKRDNPKIAD |
| | TYNYYKVFDYDVKRNNITAWEKGKTIITVKDMLKRNTPIYTRQA |
| | ACKKGELFNQTIMKKGLGQHPLKKEGPFSNISKYGGYNKVSAAY |
| | YTLIEYEEKGNKIRSLETIPLYLVKDIQKDQDVLKSYLTDLLGKKE |
| | FKILVPKIKINSLLKINGFPCHITGKTNDSFLLRPAVQFCCSNNEVL |
| | YFKKIIRFSEIRSQREKIGKTISPYEDLSFRSYIKENLWKKTKNDEIG |
| | EKEFYDLLQKKNLEIYDMLLTKHKDTIYKKRPNSATIDILVKGKE |
| | KFKSLIIENQFEVILEILKLFSATRNVSDLQHIGGSKYSGVAKIGNK |
| | ISSLDNCILIYQSITGIFEKRIDLLKV* |
| S. mutans Cas9 | MKKPYSIGLDIGTNSVGWAVVTDDYKVPAKKMKVLGNTDKSHI |
| | EKNLLGALLFDSGNTAEDRRLKRTARRRYTRRRNRILYLQEIFSE |
| | EMGKVDDSFFHRLEDSFLVTEDKRGERHPIFGNLEEEVKYHENFP |
| | TIYHLRQYLADNPEKVDLRLVYLALAHIIKFRGHFLIEGKFDTRN |
| | NDVQRLFQEFLAVYDNTFENSSLQEQNVQVEEILTDKISKSAKKD |
| | RVLKLFPNEKSNGRFAEFLKLIVGNQADFKKHFELEEKAPLQFSK |
| | DTYEEELEVLLAQIGDNYAELFLSAKKLYDSILLSGILTVTDVGTK |
| | APLSASMIQRYNEHQMDLAQLKQFIRQKLSDKYNEVFSDVSKDG |
| | YAGYIDGKTNQEAFYKYLKGLLNKIEGSGYFLDKIEREDFLRKQR |
| | TFDNGSIPHQIHLQEMRAIIRRQAEFYPFLADNQDRIEKLLTFRIPY |
| | YVGPLARGKSDFAWLSRKSADKITPWNFDEIVDKESSAEAFINRM |
| | TNYDLYLPNQKVLPKHSLLYEKFTVYNELTKVKYKTEQGKTAFF |
| | DANMKQEIFDGVFKVYRKVTKDKLMDFLEKEFDEFRIVDLTGLD |
| | KENKVFNASYGTYHDLCKILDKDFLDNSKNEKILEDIVLTLTLFE |
| | DREMIRKRLENYSDLLTKEQVKKLERRHYTGWGRLSAELIHGIR |
| | NKESRKTILDYLIDDGNSNRNFMQLINDDALSFKEEIAKAQVIGET |
| | DNLNQVVSDIAGSPAIKKGILQSLKIVDELVKIMGHQPENIVVEM |
| | ARENQFTNQGRRNSQQRLKGLTDSIKEFGSQILKEHPVENSQLQN |
| | DRLFLYYLQNGRDMYTGEELDIDYLSQYDIDHIIPQAFIKDNSIDN |
| | RVLTSSKENRGKSDDVPSKDVVRKMKSYWSKLLSAKLITQRKFD |
| | NLTKAERGGLTDDDKAGFIKRQLVETRQITKHVARILDERFNTET |
| | DENNKKIRQVKIVTLKSNLVSNFRKEFELYKVREINDYHHAHDA |
| | YLNAVIGKALLGVYPQLEPEFVYGDYPHFHGHKENKATAKKFFY |
| | SNIMNFFKKDDVRTDKNGEIIWKKDEHISNIKKVLSYPQVNIVKK |
| | VEEQTGGFSKESILPKGNSDKLIPRKTKKFYWDTKKYGGFDSPIV |
| | AYSILVIADIEKGKSKKLKTVKALVGVTIMEKMTFERDPVAFLER |
| | KGYRNVQEENIIKLPKYSLFKLENGRKRLLASARELQKGNEIVLP |
| | NHLGTLLYHAKNIHKVDEPKHLDYVDKHKDEFKELLDVVSNFSK |
| | KYTLAEGNLEKIKELYAQNNGEDLKELASSFINLLTFTAIGAPATF |
| | KFFDKNIDRKRYTSTTEILNATLIHQSITGLYETRIDLNKLGGD |
| S. thermophilus | MTKPYSIGLDIGTNSVGWAVTTDNYKVPSKKMKVLGNTSKKYIK |
| CRISPR 3 Cas9 | KNLLGVLLFDSGITAEGRRLKRTARRRYTRRRNRILYLQEIFSTEM |
| | ATLDDAFFQRLDDSFLVPDDKRDSKYPIFGNLVEEKAYHDEFPTI |
| | YHLRKYLADSTKKADLRLVYLALAHMIKYRGHFLIEGEFNSKNN |
| | DIQKNFQDFLDTYNAIFESDLSLENSKQLEEIVKDKISKLEKKDRIL |
| | KLFPGEKNSGIFSEFLKLIVGNQADFRKCFNLDEKASLHFSKESYD |
| | EDLETLLGYIGDDYSDVFLKAKKLYDAILLSGFLTVTDNETEAPL |
| | SSAMIKRYNEHKEDLALLKEYIRNISLKTYNEVFKDDTKNGYAG |
| | YIDGKTNQEDFYVYLKKLLAEFEGADYFLEKIDREDFLRKQRTFD |
| | NGSIPYQIHLQEMRAILDKQAKFYPFLAKNKERIEKILTFRIPYYV |
| | GPLARGNSDFAWSIRKRNEKITPWNFEDVIDKESSAEAFINRMTSF |
| | DLYLPEEKVLPKHSLLYETFNVYNELTKVRFIAESMRDYQFLDSK |
| | QKKDIVRLYFKDKRKVTDKDIIEYLHAIYGYDGIELKGIEKQFNSS |
| | LSTYHDLLNIINDKEFLDDSSNEAIIEEIIHTLTIFEDREMIKQRLSKF |
| | ENIFDKSVLKKLSRRHYTGWGKLSAKLINGIRDEKSGNTILDYLID |
| | DGISNRNFMQLIHDDALSFKKKIQKAQIIGDEDKGNIKEVVKSLPG |
| | SPAIKKGILQSIKIVDELVKVMGGRKPESIVVEMARENQYTNQGK |
| | SNSQQRLKRLEKSLKELGSKILKENIPAKLSKIDNNALQNDRLYL |
| | YYLQNGKDMYTGDDLDIDRLSNYDIDHIIPQAFLKDNSIDNKVLV |
| | SSASNRGKSDDVPSLEVVKKRKTFWYQLLKSKLISQRKFDNLTK |
| | AERGGLSPEDKAGFIQRQLVETRQITKHVARLLDEKFNNKKDEN |
| | NRAVRTVKIITLKSTLVSQFRKDFELYKVREINDFHHAHDAYLNA |
| | VVASALLKKYPKLEPEFVYGDYPKYNSFRERKSATEKVYFYSNI |
| | MNIFKKSISLADGRVIERPLIEVNEETGESVWNKESDLATVRRVLS |
| | YPQVNVVKKVEEQNHGLDRGKPKGLFNANLSSKPKPNSNENLV |
| | GAKEYLDPKKYGGYAGISNSFTVLVKGTIEKGAKKKITNVLEFQG |
| | ISILDRINYRKDKLNFLLEKGYKDIELIIELPKYSLFELSDGSRRML |
| | ASILSTNNKRGEIHKGNQIFLSQKFVKLLYHAKRISNTINENHRKY |
| | VENHKKEFEELFYYILEFNENYVGAKKNGKLLNSAFQSWQNHSI |
| | DELCSSFIGPTGSERKGLFELTSRGSAADFEFLGVKIPRYRDYTPSS |
| | LLKDATLIHQSVTGLYETRIDLAKLGEG |
| C. jejuni Cas9 | MARILAFDIGISSIGWAFSENDELKDCGVRIFTKVENPKTGESLAL |
| | PRRLARSARKRLARRKARLNHLKHLIANEFKLNYEDYQSFDESL |
| | AKAYKGSLISPYELRFRALNELLSKQDFARVILHIAKRRGYDDIKN |
| | SDDKEKGAILKAIKQNEEKLANYQSVGEYLYKEYFQKFKENSKE |
| | FTNVRNKKESYERCIAQSFLKDELKLIFKKQREFGFSFSKKFEEEV |
| | LSVAFYKRALKDFSHLVGNCSFFTDEKRAPKNSPLAFMFVALTRII |
| | NLLNNLKNTEGILYTKDDLNALLNEVLKNGTLTYKQTKKLLGLS |
| | DDYEFKGEKGTYFIEFKKYKEFIKALGEHNLSQDDLNEIAKDITLI |
| | KDEIKLKKALAKYDLNQNQIDSLSKLEFKDHLNISFKALKLVTPL |
| | MLEGKKYDEACNELNLKVAINEDKKDFLPAFNETYYKDEVTNPV |
| | VLRAIKEYRKVLNALLKKYGKVHKINIELAREVGKNHSQRAKIE |
| | KEQNENYKAKKDAELECEKLGLKINSKNILKLRLFKEQKEFCAYS |
| | GEKIKISDLQDEKMLEIDHIYPYSRSFDDSYMNKVLVFTKQNQEK |
| | LNQTPFEAFGNDSAKWQKIEVLAKNLPTKKQKRILDKNYKDKEQ |
| | KNFKDRNLNDTRYIARLVLNYTKDYLDFLPLSDDENTKLNDTQK |
| | GSKVHVEAKSGMLTSALRHTWGFSAKDRNNHLHHAIDAVIIAYA |
| | NNSIVKAFSDFKKEQESNSAELYAKKISELDYKNKRKFFEPFSGFR |
| | QKVLDKIDEIFVSKPERKKPSGALHEETFRKEEEFYQSYGGKEGV |
| | LKALELGKIRKVNGKIVKNGDMFRVDIFKHKKTNKFYAVPIYTM |
| | DFALKVLPNKAVARSKKGEIKDWILMDENYEFCFSLYKDSLILIQ |
| | TKDMQEPEFVYYNAFTSSTVSLIVSKHDNKFETLSKNQKILFKNA |
| | NEKEVIAKSIGIQNLKVFEKYIVSALGEVTKAEFRQREDFKK |
| P. multocida Cas9 | MQTTNLSYILGLDLGIASVGWAVVEINENEDPIGLIDVGVRIFERA |
| | EVPKTGESLALSRRLARSTRRLIRRRAHRLLLAKRFLKREGILSTID |
| | LEKGLPNQAWELRVAGLERRLSAIEWGAVLLHLIKHRGYLSKRK |
| | NESQTNNKELGALLSGVAQNHQLLQSDDYRTPAELALKKFAKEE |
| | GHIRNQRGAYTHTFNRLDLLAELNLLFAQQHQFGNPHCKEHIQQ |
| | YMTELLMWQKPALSGEAILKMLGKCTHEKNEFKAAKHTYSAER |
| | FVWLTKLNNLRILEDGAERALNEEERQLLINHPYEKSKLTYAQVR |
| | KLLGLSEQAIFKHLRYSKENAESATFMELKAWHAIRKALENQGL |
| | KDTWQDLAKKPDLLDEIGTAFSLYKTDEDIQQYLTNKVPNSVINA |
| | LLVSLNFDKFIELSLKSLRKILPLMEQGKRYDQACREIYGHHYGE |
| | ANQKTSQLLPAIPAQEIRNPVVLRTLSQARKVINAIIRQYGSPARV |
| | HIETGRELGKSFKERREIQKQQEDNRTKRESAVQKFKELFSDFSSE |
| | PKSKDILKFRLYEQQHGKCLYSGKEINIHRLNEKGYVEIDHALPFS |
| | RTWDDSFNNKVLVLASENQNKGNQTPYEWLQGKINSERWKNFV |
| | ALVLGSQCSAAKKQRLLTQVIDDNKFIDRNLNDTRYIARFLSNYI |
| | QENLLLVGKNKKNVFTPNGQITALLRSRWGLIKARENNNRHHAL |
| | DAIVVACATPSMQQKITRFIRFKEVHPYKIENRYEMVDQESGEIIS |
| | PHFPEPWAYFRQEVNIRVFDNHPDTVLKEMLPDRPQANHQFVQP |
| | LFVSRAPTRKMSGQGHMETIKSAKRLAEGISVLRIPLTQLKPNLLE |
| | NMVNKEREPALYAGLKARLAEFNQDPAKAFATPFYKQGGQQVK |
| | AIRVEQVQKSGVLVRENNGVADNASIVRTDVFIKNNKFFLVPIYT |
| | WQVAKGILPNKAIVAHKNEDEWEEMDEGAKFKFSLFPNDLVELK |
| | TKKEYFFGYYIGLDRATGNISLKEHDGEISKGKDGVYRVGVKLA |
| | LSFEKYQVDELGKNRQICRPQQRQPVR |
| F. novicida Cas9 | MNFKILPIAIDLGVKNTGVFSAFYQKGTSLERLDNKNGKVYELSK |
| | DSYTLLMNNRTARRHQRRGIDRKQLVKRLFKLIWTEQLNLEWD |
| | KDTQQAISFLFNRRGFSFITDGYSPEYLNIVPEQVKAILMDIFDDY |
| | NGEDDLDSYLKLATEQESKISEIYNKLMQKILEFKLMKLCTDIKD |
| | DKVSTKTLKEITSYEFELLADYLANYSESLKTQKFSYTDKQGNLK |
| | ELSYYFIHDKYNIQEFLKRHATINDRILDTLLTDDLDIWNFNFEKF |
| | DFDKNEEKLQNQEDKDHIQAHLHHFVFAVNKIKSEMASGGRHRS |
| | QYFQEITNVLDENNHQEGYLKNFCENLHNKKYSNLSVKNLVNLI |
| | GNLSNLELKPLRKYFNDKIHAKADHWDEQKFTETYCHWILGEW |
| | RVGVKDQDKKDGAKYSYKDLCNELKQKVTKAGLVDFLLELDPC |
| | RTIPPYLDNNNRKPPKCQSLILNPKFLDNQYPNWQQYLQELKKLQ |
| | SIQNYLDSFETDLKVLKSSKDQPYFVEYKSSNQQIASGQRDYKDL |
| | DARILQFIFDRVKASDELLLNEIYFQAKKLKQKASSELEKLESSKK |
| | LDEVIANSQLSQILKSQHTNGIFEQGTFLHLVCKYYKQRQRARDS |
| | RLYIMPEYRYDKKLHKYNNTGRFDDDNQLLTYCNHKPRQKRYQ |
| | LLNDLAGVLQVSPNFLKDKIGSDDDLFISKWLVEHIRGFKKACED |
| | SLKIQKDNRGLLNHKINIARNTKGKCEKEIFNLICKIEGSEDKKGN |
| | YKHGLAYELGVLLFGEPNEASKPEFDRKIKKFNSIYSFAQIQQIAF |
| | AERKGNANTCAVCSADNAHRMQQIKITEPVEDNKDKIILSAKAQ |
| | RLPAIPTRIVDGAVKKMATILAKNIVDDNWQNIKQVLSAKHQLHI |
| | PIITESNAFEFEPALADVKGKSLKDRRKKALERISPENIFKDKNNRI |
| | KEFAKGISAYSGANLTDGDFDGAKEELDHIIPRSHKKYGTLNDEA |
| | NLICVTRGDNKNKGNRIFCLRDLADNYKLKQFETTDDLEIEKKIA |
| | DTIWDANKKDFKFGNYRSFINLTPQEQKAFRHALFLADENPIKQA |
| | VIRAINNRNRTFVNGTQRYFAEVLANNIYLRAKKENLNTDKISFD |
| | YFGIPTIGNGRGIAEIRQLYEKVDSDIQAYAKGDKPQASYSHLIDA |
| | MLAFCIAADEHRNDGSIGLEIDKNYSLYPLDKNTGEVFTKDIFSQI |
| | KITDNEFSDKKLVRKKAIEGFNTHRQMTRDGIYAENYLPILIHKEL |
| | NEVRKGYTWKNSEEIKIFKGKKYDIQQLNNLVYCLKFVDKPISIDI |
| | QISTLEELRNILTTNNIAATAEYYYINLKTQKLHEYYIENYNTALG |
| | YKKYSKEMEFLRSLAYRSERVKIKSIDDVKQVLDKDSNFIIGKITL |
| | PFKKEWQRLYREWQNTTIKDDYEFLKSFFNVKSITKLHKKVRKD |
| | FSLPISTNEGKFLVKRKTWDNNFIYQILNDSDSRADGTKPFIPAFDI |
| | SKNEIVEAIIDSFTSKNIFWLPKNIELQKVDNKNIFAIDTSKWFEVE |
| | TPSDLRDIGIATIQYKIDNNSRPKVRVKLDYVIDDDSKINYFMNHS |
| | LLKSRYPDKVLEILKQSTIIEFESSGFNKTIKEMLGMKLAGIYNETS |
| | NN |
| Lactobacillus | MKVNNYHIGLDIGTSSIGWVAIGKDGKPLRVKGKTAIGARLFQEG |
| buchneri Cas9 | NPAADRRMFRTTRRRLSRRKWRLKLLEEIFDPYITPVDSTFFARL |
| | KQSNLSPKDSRKEFKGSMLFPDLTDMQYHKNYPTIYHLRHALMT |
| | QDKKFDIRMVYLAIHHIVKYRGNFLNSTPVDSFKASKVDFVDQF |
| | KKLNELYAAINPEESFKINLANSEDIGHQFLDPSIRKFDKKKQIPKI |
| | VPVMMNDKVTDRLNGKIASEIIHAILGYKAKLDVVLQCTPVDSK |
| | PWALKFDDEDIDAKLEKILPEMDENQQSIVAILQNLYSQVTLNQI |
| | VPNGMSLSESMIEKYNDHHDHLKLYKKLIDQLADPKKKAVLKK |
| | AYSQYVGDDGKVIEQAEFWSSVKKNLDDSELSKQIMDLIDAEKF |
| | MPKQRTSQNGVIPHQLHQRELDEIIEHQSKYYPWLVEINPNKHDL |
| | HLAKYKIEQLVAFRVPYYVGPMITPKDQAESAETVFSWMERKGT |
| | ETGQITPWNFDEKVDRKASANRFIKRMTTKDTYLIGEDVLPDESL |
| | LYEKFKVLNELNMVRVNGKLLKVADKQAIFQDLFENYKHVSVK |
| | KLQNYIKAKTGLPSDPEISGLSDPEHFNNSLGTYNDFKKLFGSKV |
| | DEPDLQDDFEKIVEWSTVFEDKKILREKLNEITWLSDQQKDVLES |
| | SRYQGWGRLSKKLLTGIVNDQGERIIDKLWNTNKNFMQIQSDDD |
| | FAKRIHEANADQMQAVDVEDVLADAYTSPQNKKAIRQVVKVVD |
| | DIQKAMGGVAPKYISIEFTRSEDRNPRRTISRQRQLENTLKDTAKS |
| | LAKSINPELLSELDNAAKSKKGLTDRLYLYFTQLGKDIYTGEPINI |
| | DELNKYDIDHILPQAFIKDNSLDNRVLVLTAVNNGKSDNVPLRMF |
| | GAKMGHFWKQLAEAGLISKRKLKNLQTDPDTISKYAMHGFIRRQ |
| | LVETSQVIKLVANILGDKYRNDDTKIIEITARMNHQMRDEFGFIK |
| | NREINDYHHAFDAYLTAFLGRYLYHRYIKLRPYFVYGDFKKFRE |
| | DKVTMRNFNFLHDLTDDTQEKIADAETGEVIWDRENSIQQLKDV |
| | YHYKFMLISHEVYTLRGAMFNQTVYPASDAGKRKLIPVKADRPV |
| | NVYGGYSGSADAYMAIVRIHNKKGDKYRVVGVPMRALDRLDA |
| | AKNVSDADFDRALKDVLAPQLTKTKKSRKTGEITQVIEDFEIVLG |
| | KVMYRQLMIDGDKKFMLGSSTYQYNAKQLVLSDQSVKTLASKG |
| | RLDPLQESMDYNNVYTEILDKVNQYFSLYDMNKFRHKLNLGFSK |
| | FISFPNHNVLDGNTKVSSGKREILQEILNGLHANPTFGNLKDVGIT |
| | TPFGQLQQPNGILLSDETKIRYQSPTGLFERTVSLKDL |
| Listeria innocua | MKKPYTIGLDIGTNSVGWAVLTDQYDLVKRKMKIAGDSEKKQIK |
| Cas9 | KNFWGVRLFDEGQTAADRRMARTARRRIERRRNRISYLQGIFAE |
| | EMSKTDANFFCRLSDSFYVDNEKRNSRHPFFATIEEEVEYHKNYP |
| | TIYHLREELVNSSEKADLRLVYLALAHIIKYRGNFLIEGALDTQNT |
| | SVDGIYKQFIQTYNQVFASGIEDGSLKKLEDNKDVAKILVEKVTR |
| | KEKLERILKLYPGEKSAGMFAQFISLIVGSKGNFQKPFDLIEKSDIE |
| | CAKDSYEEDLESLLALIGDEYAELFVAAKNAYSAVVLSSIITVAET |
| | ETNAKLSASMIERFDTHEEDLGELKAFIKLHLPKHYEEIFSNTEKH |
| | GYAGYIDGKTKQADFYKYMKMTLENIEGADYFIAKIEKENFLRK |
| | QRTFDNGAIPHQLHLEELEAILHQQAKYYPFLKENYDKIKSLVTF |
| | RIPYFVGPLANGQSEFAWLTRKADGEIRPWNIEEKVDFGKSAVDF |
| | IEKMTNKDTYLPKENVLPKHSLCYQKYLVYNELTKVRYINDQGK |
| | TSYFSGQEKEQIFNDLFKQKRKVKKKDLELFLRNMSHVESPTIEG |
| | LEDSFNSSYSTYHDLLKVGIKQEILDNPVNTEMLENIVKILTVFED |
| | KRMIKEQLQQFSDVLDGVVLKKLERRHYTGWGRLSAKLLMGIR |
| | DKQSHLTILDYLMNDDGLNRNLMQLINDSNLSFKSIIEKEQVTTA |
| | DKDIQSIVADLAGSPAIKKGILQSLKIVDELVSVMGYPPQTIVVEM |
| | ARENQTTGKGKNNSRPRYKSLEKAIKEFGSQILKEHPTDNQELRN |
| | NRLYLYYLQNGKDMYTGQDLDIHNLSNYDIDHIVPQSFITDNSID |
| | NLVLTSSAGNREKGDDVPPLEIVRKRKVFWEKLYQGNLMSKRKF |
| | DYLTKAERGGLTEADKARFIHRQLVETRQITKNVANILHQRFNYE |
| | KDDHGNTMKQVRIVTLKSALVSQFRKQFQLYKVRDVNDYHHAH |
| | DAYLNGVVANTLLKVYPQLEPEFVYGDYHQFDWFKANKATAK |
| | KQFYTNIMLFFAQKDRIIDENGEILWDKKYLDTVKKVMSYRQMN |
| | IVKKTEIQKGEFSKATIKPKGNSSKLIPRKTNWDPMKYGGLDSPN |
| | MAYAVVIEYAKGKNKLVFEKKIIRVTIMERKAFEKDEKAFLEEQ |
| | GYRQPKVLAKLPKYTLYECEEGRRRMLASANEAQKGNQQVLPN |
| | HLVTLLHHAANCEVSDGKSLDYIESNREMFAELLAHVSEFAKRY |
| | TLAEANLNKINQLFEQNKEGDIKAIAQSFVDLMAFNAMGAPASF |
| | KFFETTIERKRYNNLKELLNSTIIYQSITGLYESRKRLDD |
| L. pneumophiha | MESSQILSPIGIDLGGKFTGVCLSHLEAFAELPNHANTKYSVILIDH |
| Cas9 | NNFQLSQAQRRATRHRVRNKKRNQFVKRVALQLFQHILSRDLNA |
| | KEETALCHYLNNRGYTYVDTDLDEYIKDETTINLLKELLPSESEH |
| | NFIDWFLQKMQSSEFRKILVSKVEEKKDDKELKNAVKNIKNFITG |
| | FEKNSVEGHRHRKVYFENIKSDITKDNQLDSIKKKIPSVCLSNLLG |
| | HLSNLQWKNLHRYLAKNPKQFDEQTFGNEFLRMLKNFRHLKGS |
| | QESLAVRNLIQQLEQSQDYISILEKTPPEITIPPYEARTNTGMEKDQ |
| | SLLLNPEKLNNLYPNWRNLIPGIIDAHPFLEKDLEHTKLRDRKRIIS |
| | PSKQDEKRDSYILQRYLDLNKKIDKFKIKKQLSFLGQGKQLPANLI |
| | ETQKEMETHFNSSLVSVLIQIASAYNKEREDAAQGIWFDNAFSLC |
| | ELSNINPPRKQKILPLLVGAILSEDFINNKDKWAKFKIFWNTHKIG |
| | RTSLKSKCKEIEEARKNSGNAFKIDYEEALNHPEHSNNKALIKIIQ |
| | TIPDIIQAIQSHLGHNDSQALIYHNPFSLSQLYTILETKRDGFHKNC |
| | VAVTCENYWRSQKTEIDPEISYASRLPADSVRPFDGVLARMMQR |
| | LAYEIAMAKWEQIKHIPDNSSLLIPIYLEQNRFEFEESFKKIKGSSS |
| | DKTLEQAIEKQNIQWEEKFQRIINASMNICPYKGASIGGQGEIDHI |
| | YPRSLSKKHFGVIFNSEVNLIYCSSQGNREKKEEHYLLEHLSPLYL |
| | KHQFGTDNVSDIKNFISQNVANIKKYISFHLLTPEQQKAARHALFL |
| | DYDDEAFKTITKFLMSQQKARVNGTQKFLGKQIMEFLSTLADSK |
| | QLQLEFSIKQITAEEVHDHRELLSKQEPKLVKSRQQSFPSHAIDAT |
| | LTMSIGLKEFPQFSQELDNSWFINHLMPDEVHLNPVRSKEKYNKP |
| | NISSTPLFKDSLYAERFIPVWVKGETFAIGFSEKDLFEIKPSNKEKL |
| | FTLLKTYSTKNPGESLQELQAKSKAKWLYFPINKTLALEFLHHYF |
| | HKEIVTPDDTTVCHFINSLRYYTKKESITVKILKEPMPVLSVKFESS |
| | KKNVLGSFKHTIALPATKDWERLFNHPNFLALKANPAPNPKEFNE |
| | FIRKYFLSDNNPNSDIPNNGHNIKPQKHKAVRKVFSLPVIPGNAGT |
| | MMRIRRKDNKGQPLYQLQTIDDTPSMGIQINEDRLVKQEVLMDA |
| | YKTRNLSTIDGINNSEGQAYATFDNWLTLPVSTFKPEIIKLEMKPH |
| | SKTRRYIRITQSLADFIKTIDEALMIKPSDSIDDPLNMPNEIVCKNK |
| | LFGNELKPRDGKMKIVSTGKIVTYEFESDSTPQWIQTLYVTQLKK |
| | QP |
| N. lactamica Cas9 | MAAFKPNPMNYILGLDIGIASVGWAMVEVDEEENPIRLIDLGVRV |
| | FERAEVPKTGDSLAMARRLARSVRRLTRRRAHRLLRARRLLKRE |
| | GVLQDADFDENGLVKSLPNTPWQLRAAALDRKLTCLEWSAVLL |
| | HLVKHRGYLSQRKNEGETADKELGALLKGVADNAHALQTGDFR |
| | TPAELALNKFEKESGHIRNQRGDYSHTFSRKDLQAELNLLFEKQK |
| | EFGNPHVSDGLKEDIETLLMAQRPALSGDAVQKMLGHCTFEPAE |
| | PKAAKNTYTAERFIWLTKLNNLRILEQGSERPLTDTERATLMDEP |
| | YRKSKLTYAQARKLLGLEDTAFFKGLRYGKDNAEASTLMEMKA |
| | YHAISRALEKEGLKDKKSPLNLSTELQDEIGTAFSLFKTDKDITGR |
| | LKDRVQPEILEALLKHISFDKFVQISLKALRRIVPLMEQGKRYDEA |
| | CAEIYGDHYCKKNAEEKIYLPPIPADEIRNPVVLRALSQARKVINC |
| | VVRRYGSPARIHIETAREVGKSFKDRKEIEKRQEENRKDREKAAA |
| | KFREYFPNFVGEPKSKDILKLRLYEQQHGKCLYSGKEINLVRLNE |
| | KGYVEIDHALPFSRTWDDSFNNKVLVLGSENQNKGNQTPYEYFN |
| | GKDNSREWQEFKARVETSRFPRSKKQRILLQKFDEEGFKERNLN |
| | DTRYVNRFLCQFVADHILLTGKGKRRVFASNGQITNLLRGFWGL |
| | RKVRTENDRHHALDAVVVACSTVAMQQKITRFVRYKEMNAFDG |
| | KTIDKETGEVLHQKAHFPQPWEFFAQEVMIRVFGKPDGKPEFEEA |
| | DTPEKLRTLLAEKLSSRPEAVHEYVTPLFVSRAPNRKMSGQGHM |
| | ETVKSAKRLDEGISVLRVPLTQLKLKGLEKMVNREREPKLYDAL |
| | KAQLETHKDDPAKAFAEPFYKYDKAGSRTQQVKAVRIEQVQKT |
| | GVWVRNHNGIADNATMVRVDVFEKGGKYYLVPIYSWQVAKGIL |
| | PDRAVVAFKDEEDWTVMDDSFEFRFVLYANDLIKLTAKKNEFLG |
| | YFVSLNRATGAIDIRTHDTDSTKGKNGIFQSVGVKTALSFQKNQI |
| | DELGKEIRPCRLKKRPPVR |
| N. meningitides | MAAFKPNPINYILGLDIGIASVGWAMVEIDEDENPICLIDLGVRVF |
| Cas9 | ERAEVPKTGDSLAMARRLARSVRRLTRRRAHRLLRARRLLKREG |
| | VLQAADFDENGLIKSLPNTPWQLRAAALDRKLTPLEWSAVLLHLI |
| | KHRGYLSQRKNEGETADKELGALLKGVADNAHALQTGDFRTPA |
| | ELALNKFEKESGHIRNQRGDYSHTFSRKDLQAELILLFEKQKEFG |
| | NPHVSGGLKEGIETLLMTQRPALSGDAVQKMLGHCTFEPAEPKA |
| | AKNTYTAERFIWLTKLNNLRILEQGSERPLTDTERATLMDEPYRK |
| | SKLTYAQARKLLGLEDTAFFKGLRYGKDNAEASTLMEMKAYHA |
| | ISRALEKEGLKDKKSPLNLSPELQDEIGTAFSLFKTDEDITGRLKD |
| | RIQPEILEALLKHISFDKFVQISLKALRRIVPLMEQGKRYDEACAEI |
| | YGDHYGKKNTEEKIYLPPIPADEIRNPVVLRALSQARKVINGVVR |
| | RYGSPARIHIETAREVGKSFKDRKEIEKRQEENRKDREKAAAKFR |
| | EYFPNFVGEPKSKDILKLRLYEQQHGKCLYSGKEINLGRLNEKGY |
| | VEIDHALPFSRTWDDSFNNKVLVLGSENQNKGNQTPYEYFNGKD |
| | NSREWQEFKARVETSRFPRSKKQRILLQKFDEDGFKERNLNDTRY |
| | VNRFLCQFVADRMRLTGKGKKRVFASNGQITNLLRGFWGLRKV |
| | RAENDRHHALDAVVVACSTVAMQQKITRFVRYKEMNAFDGKTI |
| | DKETGEVLHQKTHFPQPWEFFAQEVMIRVFGKPDGKPEFEEADT |
| | PEKLRTLLAEKLSSRPEAVHEYVTPLFVSRAPNRKMSGQGHMET |
| | VKSAKRLDEGVSVLRVPLTQLKLKDLEKMVNREREPKLYEALKA |
| | RLEAHKDDPAKAFAEPFYKYDKAGNRTQQVKAVRVEQVQKTG |
| | VWVRNHNGIADNATMVRVDVFEKGDKYYLVPIYSWQVAKGILP |
| | DRAVVQGKDEEDWQLIDDSFNFKFSLHPNDLVEVITKKARMFGY |
| | FASCHRGTGNINIRIHDLDHKIGKNGILEGIGVKTALSFQKYQIDEL |
| | GKEIRPCRLKKRPPVR |
| B. longum Cas9 | MLSRQLLGASHLARPVSYSYNVQDNDVHCSYGERCFMRGKRYR |
| | IGIDVGLNSVGLAAVEVSDENSPVRLLNAQSVIHDGGVDPQKNKE |
| | AITRKNMSGVARRTRRMRRRKRERLHKLDMLLGKFGYPVIEPES |
| | LDKPFEEWHVRAELATRYIEDDELRRESISIALRHMARHRGWRNP |
| | YRQVDSLISDNPYSKQYGELKEKAKAYNDDATAAEEESTPAQLV |
| | VAMLDAGYAEAPRLRWRTGSKKPDAEGYLPVRLMQEDNANEL |
| | KQIFRVQRVPADEWKPLFRSVFYAVSPKGSAEQRVGQDPLAPEQ |
| | ARALKASLAFQEYRIANVITNLRIKDASAELRKLTVDEKQSIYDQ |
| | LVSPSSEDITWSDLCDFLGFKRSQLKGVGSLTEDGEERISSRPPRLT |
| | SVQRIYESDNKIRKPLVAWWKSASDNEHEAMIRLLSNTVDIDKV |
| | REDVAYASAIEFIDGLDDDALTKLDSVDLPSGRAAYSVETLQKLT |
| | RQMLTTDDDLHEARKTLFNVTDSWRPPADPIGEPLGNPSVDRVL |
| | KNVNRYLMNCQQRWGNPVSVNIEHVRSSFSSVAFARKDKREYE |
| | KNNEKRSIFRSSLSEQLRADEQMEKVRESDLRRLEAIQRQNGQCL |
| | YCGRTITFRTCEMDHIVPRKGVGSTNTRTNFAAVCAECNRMKSN |
| | TPFAIWARSEDAQTRGVSLAEAKKRVTMFTFNPKSYAPREVKAF |
| | KQAVIARLQQTEDDAAIDNRSIESVAWMADELHRRIDWYFNAKQ |
| | YVNSASIDDAEAETMKTTVSVFQGRVTASARRAAGIEGKIHFIGQ |
| | QSKTRLDRRHHAVDASVIAMMNTAAAQTLMERESLRESQRLIGL |
| | MPGERSWKEYPYEGTSRYESFHLWLDNMDVLLELLNDALDNDR |
| | IAVMQSQRYVLGNSIAHDATIHPLEKVPLGSAMSADLIRRASTPA |
| | LWCALTRLPDYDEKEGLPEDSHREIRVHDTRYSADDEMGFFASQ |
| | AAQIAVQEGSADIGSAIHHARVYRCWKTNAKGVRKYFYGMIRVF |
| | QTDLLRACHDDLFTVPLPPQSISMRYGEPRVVQALQSGNAQYLG |
| | SLVVGDEIEMDFSSLDVDGQIGEYLQFFSQFSGGNLAWKHWVVD |
| | GFFNQTQLRIRPRYLAAEGLAKAFSDDVVPDGVQKIVTKQGWLP |
| | PVNTASKTAVRIVRRNAFGEPRLSSAHHMPCSWQWRHE |
| A. muciniphila Cas9 | MSRSLTFSFDIGYASIGWAVIASASHDDADPSVCGCGTVLFPKDD |
| | CQAFKRREYRRLRRNIRSRRVRIERIGRLLVQAQIITPEMKETSGH |
| | PAPFYLASEALKGHRTLAPIELWHVLRWYAHNRGYDNNASWSN |
| | SLSEDGGNGEDTERVKHAQDLMDKHGTATMAETICRELKLEEG |
| | KADAPMEVSTPAYKNLNTAFPRLIVEKEVRRILELSAPLIPGLTAEI |
| | IELIAQHHPLTTEQRGVLLQHGIKLARRYRGSLLFGQLIPRFDNRII |
| | SRCPVTWAQVYEAELKKGNSEQSARERAEKLSKVPTANCPEFYE |
| | YRMARILCNIRADGEPLSAEIRRELMNQARQEGKLTKASLEKAIS |
| | SRLGKETETNVSNYFTLHPDSEEALYLNPAVEVLQRSGIGQILSPS |
| | VYRIAANRLRRGKSVTPNYLLNLLKSRGESGEALEKKIEKESKKK |
| | EADYADTPLKPKYATGRAPYARTVLKKVVEEILDGEDPTRPARG |
| | EAHPDGELKAHDGCLYCLLDTDSSVNQHQKERRLDTMTNNHLV |
| | RHRMLILDRLLKDLIQDFADGQKDRISRVCVEVGKELTTFSAMDS |
| | KKIQRELTLRQKSHTDAVNRLKRKLPGKALSANLIRKCRIAMDM |
| | NWTCPFTGATYGDHELENLELEHIVPHSFRQSNALSSLVLTWPGV |
| | NRMKGQRTGYDFVEQEQENPVPDKPNLHICSLNNYRELVEKLDD |
| | KKGHEDDRRRKKKRKALLMVRGLSHKHQSQNHEAMKEIGMTE |
| | GMMTQSSHLMKLACKSIKTSLPDAHIDMIPGAVTAEVRKAWDVF |
| | GVFKELCPEAADPDSGKILKENLRSLTHLHHALDACVLGLIPYIIP |
| | AHHNGLLRRVLAMRRIPEKLIPQVRPVANQRHYVLNDDGRMML |
| | RDLSASLKENIREQLMEQRVIQHVPADMGGALLKETMQRVLSVD |
| | GSGEDAMVSLSKKKDGKKEKNQVKASKLVGVFPEGPSKLKALK |
| | AAIEIDGNYGVALDPKPVVIRHIKVFKRIMALKEQNGGKPVRILK |
| | KGMLIHLTSSKDPKHAGVWRIESIQDSKGGVKLDLQRAHCAVPK |
| | NKTHECNWREVDLISLLKKYQMKRYPTSYTGTPR |
| O. laneus Cas9 | METTLGIDLGTNSIGLALVDQEEHQILYSGVRIFPEGINKDTIGLGE |
| | KEESRNATRRAKRQMRRQYFRKKLRKAKLLELLIAYDMCPLKPE |
| | DVRRWKNWDKQQKSTVRQFPDTPAFREWLKQNPYELRKQAVT |
| | EDVTRPELGRILYQMIQRRGFLSSRKGKEEGKIFTGKDRMVGIDE |
| | TRKNLQKQTLGAYLYDIAPKNGEKYRFRTERVRARYTLRDMYIR |
| | EFEIIWQRQAGHLGLAHEQATRKKNIFLEGSATNVRNSKLITHLQ |
| | AKYGRGHVLIEDTRITVTFQLPLKEVLGGKIEIEEEQLKFKSNESV |
| | LFWQRPLRSQKSLLSKCVFEGRNFYDPVHQKWIIAGPTPAPLSHP |
| | EFEEFRAYQFINNIIYGKNEHLTAIQREAVFELMCTESKDFNFEKIP |
| | KHLKLFEKFNFDDTTKVPACTTISQLRKLFPHPVWEEKREEIWHC |
| | FYFYDDNTLLFEKLQKDYALQTNDLEKIKKIRLSESYGNVSLKAI |
| | RRINPYLKKGYAYSTAVLLGGIRNSFGKRFEYFKEYEPEIEKAVC |
| | RILKEKNAEGEVIRKIKDYLVHNRFGFAKNDRAFQKLYHHSQAIT |
| | TQAQKERLPETGNLRNPIVQQGLNELRRTVNKLLATCREKYGPSF |
| | KFDHIHVEMGRELRSSKTEREKQSRQIRENEKKNEAAKVKLAEY |
| | GLKAYRDNIQKYLLYKEIEEKGGTVCCPYTGKTLNISHTLGSDNS |
| | VQIEHIIPYSISLDDSLANKTLCDATFNREKGELTPYDFYQKDPSPE |
| | KWGASSWEEIEDRAFRLLPYAKAQRFIRRKPQESNEFISRQLNDT |
| | RYISKKAVEYLSAICSDVKAFPGQLTAELRHLWGLNNILQSAPDIT |
| | FPLPVSATENHREYYVITNEQNEVIRLFPKQGETPRTEKGELLLTG |
| | EVERKVFRCKGMQEFQTDVSDGKYWRRIKLSSSVTWSPLFAPKPI |
| | SADGQIVLKGRIEKGVFVCNQLKQKLKTGLPDGSYWISLPVISQT |
| | FKEGESVNNSKLTSQQVQLFGRVREGIFRCHNYQCPASGADGNF |
| | WCTLDTDTAQPAFTPIKNAPPGVGGGQIILTGDVDDKGIFHADDD |
| | LHYELPASLPKGKYYGIFTVESCDPTLIPIELSAPKTSKGENLIEGNI |
| | WVDEHTGEVRFDPKKNREDQRHHAIDAIVIALSSQSLFQRLSTYN |
| | ARRENKKRGLDSTEHFPSPWPGFAQDVRQSVVPLLVSYKQNPKT |
| | LCKISKTLYKDGKKIHSCGNAVRGQLHKETVYGQRTAPGATEKS |
| | YHIRKDIRELKTSKHIGKVVDITIRQMLLKHLQENYHIDITQEFNIP |
| | SNAFFKEGVYRIFLPNKHGEPVPIKKIRMKEELGNAERLKDNINQ |
| | YVNPRNNHHVMIYQDADGNLKEEIVSFWSVIERQNQGQPIYQLP |
| | REGRNIVSILQINDTFLIGLKEEEPEVYRNDLSTLSKHLYRVQKLS |
| | GMYYTFRHHLASTLNNEREEFRIQSLEAWKRANPVKVQIDEIGRI |
| | TFLNGPLC |

The term “cell” as used herein may refer to either a prokaryotic or eukaryotic cell, optionally obtained from a subject or a commercially available source.

As used herein, the term “CRISPR” refers to Clustered Regularly Interspaced Short Palindromic Repeats (CRISPR). CRISPR may also refer to a technique or system of sequence-specific genetic manipulation relying on the CRISPR pathway. A CRISPR recombinant expression system can be programmed to cleave a target polynucleotide using a CRISPR endonuclease and a guideRNA or a combination of a crRNA and a tracrRNA. A CRISPR system can be used to cause double stranded or single stranded breaks in a target polynucleotide such as DNA or RNA. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359 and Hsu et al. (2014) Cell 156(6): 1262-1278.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure. The term “encode” as it is applied to nucleic acid sequences refers to a polynucleotide which is said to “encode” a polypeptide, an mRNA, or an effector RNA if, in its native state or when manipulated by methods well known to those skilled in the art, can be transcribed and/or translated to produce the effector RNA, the mRNA, or an mRNA that can for the polypeptide and/or a fragment thereof. The antisense strand is the complement of such a nucleic acid, and the encoding sequence can be deduced therefrom.

As used herein, the term “expression” or “gene expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260, each incorporated herein in their entirety. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some embodiments, a gRNA is synthetic (Kelley, M. et al. (2016) J of Biotechnology 233 (2016) 74-83, incorporated by reference herein in its entirety). In some embodiments, a gRNA is engineered to have one or more modifications that improve specificity, binding, or other features of the gRNA. In some embodiments, a gRNA is an enhanced gRNA (“esgRNA”) (Chen B, et al. Cell. 2013; 155:1479-1491. doi: 10.1016/j.cell.2013.12.001, incorporated by reference herein in its entirety).

The term “intein” refers to a class of protein that is able to excise itself and join the remaining portion(s) of the protein via protein splicing. A “split intein” comes from two genes. A non-limiting example of a “split-intein” are the C-intein and N-intein sequences originally derived from N. punctiforme.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “ortholog” is used in reference of another gene or protein and intends a homolog of said gene or protein that evolved from the same ancestral source. Orthologs may or may not retain the same function as the gene or protein to which they are orthologous. Non-limiting examples of Cas9 orthologs include S. aureus Cas9 (“spCas9”), S. thermophiles Cas9, L. pneumophilia Cas9, N. lactamica Cas9, N. meningitides Cas9, B. longum Cas9, A. muciniphila Cas9, and O. laneus Cas9.

The term “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nP2, PPE, ENK, EAAT2, GFAP, MBP, and U6 promoters. An “enhancer” is a region of DNA that can be bound by activating proteins to increase the likelihood or frequency of transcription. Non-limiting exemplary enhancers and posttranscriptional regulatory elements include the CMV enhancer and WPRE.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “recombinant expression system” refers to a genetic construct for the expression of certain genetic material formed by recombination.

As used herein, the term “RNA methylation” refers to an RNA molecule comprising at least one ribonucleotide modified with one or more methyl groups. Non-limiting examples of RNA methylation include but are not limited to N⁶-methyladenosine (m⁶A), N¹-methyladenosine (m¹A), N⁷-methyladenosine (m⁷A), N⁷-methylguanosine (m⁷G), 5-methylcytosine (m⁵C), N6,2-O dimethyladenosinez (m⁶Am), and 2′-O-methylation (2′OMe). In particular embodiments, RNA methylation refers to m⁶A methylation. m⁶A is one of the most abundant forms of RNA methylation and plays a vital role in regulating gene expression, protein translation, cell behaviors, and physiological conditions in many species, including humans. m⁶A is increasingly recognized for its ability to functionally modulates the eukaryotic transcriptome to influence mRNA splicing, export, localization, translation, and stability (Du, K. et al. Mol Neurobiol. 2018 Jun. 16. doi: 10.1007/s12035-018-1138-1, incorporated herein in its entirety by reference). In some embodiments, an m6A site is found within the consensus sequence Rm6ACH (R=G or A, H=A, C, or U) of a target RNA.

As used herein, the term “RNA methylation modification protein” or “RMMP” refers to a polypeptide capable of modulating RNA methylation of a target RNA. In some embodiments, the RMMP comprises a polypeptide with writer, reader, or eraser function. For example, the dynamic and reversible modification of m⁶A is conducted by three elements: methyltransferases (“writers”), such as methyltransferase-like protein 3 (METTL3) and METTL14; m⁶A-binding proteins (“readers”), such as the YTH domain family proteins (YTHDFs) and YTH domain-containing protein 1 (YTHDC1); and demethylases (“erasers”), such as fat mass and obesity-associated protein (FTO) and AlkB homolog 5 (ALKBH5). In some embodiments, the RMMP is specific for the m⁶A modification. In some embodiments, the RMMP is all or part of N6-adenosine-methyltransferase 70 kDa subunit (METTL3), Methyltransferase like 14 (METTL14), Methyltransferase like 16 (METTL16), Wilms tumor 1 associated protein (WTAP), AlkB homolog 5, RNA demelthylase (ALKBH5), Fat mass and obesity-associated protein (FTO), and a biological equivalent of each thereof.

As used herein, the term “subject” is intended to mean any eukaryotic organism such as a plant or an animal. In some embodiments, the subject may be a mammal; in further embodiments, the subject may be a bovine, equine, feline, murine, porcine, canine, human, or rat.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, the term “vector” intends a recombinant vector that retains the ability to infect and transduce non-dividing and/or slowly-dividing cells and integrate into the target cell's genome. The vector may be derived from or based on a wild-type virus. Aspects of this disclosure relate to an adeno-associated virus vector, an adenovirus vector, and a lentivirus vector.

As used herein, the term “XTEN linker” intends a polypeptide comprising six amino acids repeats (Gly, Ala, Pro, Glu, Ser, Thr). In some embodiments, fusion of an XTEN linker to a protein reduces the rate of clearance and degradation of the fusion protein. In some embodiments, the XTEN linker is unstructured.

It is to be inferred without explicit recitation and unless otherwise intended, that when the present disclosure relates to a polypeptide, protein, polynucleotide or antibody, an equivalent or a biologically equivalent of such is intended within the scope of this disclosure.

As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement. In some embodiments, a biological equivalent retains the

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Modes of Carrying out the Disclosure

Described herein are compositions, kits, systems, and methods useful to perform programmable RNA modification at single-nucleotide resolution using RNA-targeting CRISPR/Cas: single guide RNA combinations. In some embodiments, compositions, kits, systems, and methods described herein employ an effector enzyme. Exemplary effector enzymes include, without limitation, RMMPs and enzymes with cytidine deaminase activity.

In some embodiments, described herein are compositions, kits, systems, and methods useful to perform programmable RNA m⁶A modification at single-nucleotide resolution using RNA-targeting CRISPR/Cas: single guide RNA combinations. This approach, termed ‘Cas-directed RNA m⁶A modification’, provides a means to reversibly alter genetic information in a temporal manner, unlike traditional CRISPR/Cas9 driven genomic engineering which relies on permanently altering DNA sequence. This disclosure stems from taking a nuclease-dead version of DNA/RNA-targeting Cas (e.g., Sp/Sau/Cje dCas9 or dCas13a/b/d) and generating recombinant proteins with effector enzymes capable of performing ribonucleotide base modification to alter how sequence of the RNA molecule is recognized by cellular machinery. Specifically, the inventors have made constructs that express RNA-targeting Cas (for example dCas9 or dCas13b/d) fused to the open reading frames of human METTL3, METTL14, METTL16, WTAP or FTO) or combinations of reading frames of these proteins, using a linker for spatial separation. With RNA-targeting Cas as a surrogate RNA-binding motif, the compositions, kits, systems, and methods described herein can be used to direct m⁶A modification to specific RNA sites for modification.

N⁶-methyladenosine (m⁶A) RNA methylation is one of the most prevalent modifications of RNA, accounting for about 50% of total methylated ribonucleotides and 0.1-0.4% of all adenosines in total cellular RNAs. The biological function of m⁶A RNA methylation is highly variable depending on context and little is known about the underlying mechanisms. However, emerging evidence has suggested that m⁶A modification plays a pivotal role in pre-mRNA splicing, 3′-end processing, nuclear export, translation regulation, mRNA decay, and miRNA processing.

In some embodiments, described herein are compositions, kits, systems, and methods useful to perform programmable cytidine to uridine conversions of RNA (e.g., using an enzyme that has cytidine deaminase activity). This disclosure stems from taking a nuclease-dead version of DNA/RNA-targeting Cas (e.g., Sp/Sau/Cje dCas9 or dCas13a/b/d) and generating recombinant proteins with effector enzymes capable of performing C to U conversions. Specifically, the inventors have made constructs that express RNA-targeting Cas (for example dCas9 or dCas13b/d) fused to the open reading frames of human APOBEC. With RNA-targeting Cas as a surrogate RNA-binding motif, the compositions, kits, systems, and methods described herein can be used to direct C-to-U conversions at specific RNA sites.

### Fusion Proteins

Provided herein are fusion proteins comprising, consisting of, or consisting essentially of: (i) a guide nucleotide sequence-programmable RNA binding protein; and (ii) an effector enzyme. Exemplary effector enzymes include, without limitation, RMMPs and enzymes with cytidine deaminase activity.

In some aspects, provided herein are fusion proteins comprising, consisting of, or consisting essentially of: (i) a guide nucleotide sequence-programmable RNA binding protein; and (ii) a RNA methylation modification protein (RMMP) or a biological equivalent thereof. In some embodiments, the RMMP comprises a polypeptide with writer, reader, or eraser function. In some embodiments, the RBPM is m6A specific. In some embodiments, the RMMP is all or part of N6-adenosine-methyltransferase 70 kDa subunit (METTL3), Methyltransferase like 14 (METTL14), Methyltransferase like 16 (METTL16), Wilms tumor 1 associated protein (WTAP), AlkB homolog 5, RNA demelthylase (ALKBH5), Fat mass and obesity-associated protein (FTO), and a biological equivalent of each thereof.

In some aspects, provided herein are fusion proteins comprising, consisting of, or consisting essentially of: (i) a guide nucleotide sequence-programmable RNA binding protein; and (ii) enzymes with cytidine deaminase activity. The enzymes with cytidine deaminase activity can catalyze C-to-U conversions in a target RNA. The enzymes with cytidine deaminase activity can be, e.g., an Apolipoprotein B mRNA editing enzyme, catalytic polypeptide 1 (Apobec-1). Apobec-1 related genes that feature cytidine deaminase active sites, including Apobec-2/ARCD1, activation-induced deaminase (AID), and phorbolins/ARCD2-7/apobec-3, are also contemplated (See, e.g., Blanc and Davidson, J Biol Chem, 278(3):1395-8, 2003).

In some embodiments, the guide nucleotide sequence-programmable RNA binding protein is all or part of a protein selected from: Cas9, modified Cas9, Cas13a, Cas13b, CasRX/Cas13d, and a biological equivalent of each thereof. In some embodiments, the guide nucleotide sequence-programmable RNA binding protein is all or part of a protein selected from: Steptococcus pyogenes Cas9 (spCas9), Staphilococcus aureus Cas9 (saCas9), Francisella novicida Cas9 (FnCas9), Neisseria meningitidis Cas9 (nmCas9), Streptococcus thermophilus CRISPR 1 Cas9 (St1Cas9), Streptococcus thermophilus CRISPR 3 Cas9 (St3Cas9), and Brevibacillus laterosporus Cas9 (BlatCas9). In some embodiments, the guide nucleotide sequence-programmable RNA binding protein is modified to be nuclease inactive. In some embodiments, the fusion protein further comprises, consists of, or consists essentially of a linker. In some embodiments, the linker is a peptide linker. In some embodiments, the peptide linker comprises one or more repeats of the tri-peptide GGS. In some embodiments, the linker is an XTEN linker. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, poly cyanoacrylates, lipid polymers, chitins, hyaluronic acid, heparin, or an alkyl linker. In some embodiments, the components of the fusion protein are fused via intein-mediated fusion.

In some embodiments, the fusion protein comprises, consists of, or consists essentially of the structure the structure NH₂-[effector enzyme]-[linker]-[guide nucleotide sequence-programmable RNA binding protein], or the structure NH₂-[guide nucleotide sequence-programmable RNA binding protein]-[linker]-[effector enzyme]. In some embodiments, the fusion protein comprises, consists of, or consists essentially of the structure NH₂-[RMMP]-[linker]-[guide nucleotide sequence-programmable RNA binding protein]-COOH. In other embodiments, the fusion protein comprises, consists of, or consists essentially of the structure NH₂-[guide nucleotide sequence-programmable RNA binding protein]-[linker]-[RMMP]—COOH. In some embodiments, the fusion protein comprises, consists of, or consists essentially of the structure NH₂-[enzyme with cytidine deaminase activity]-[linker]-[guide nucleotide sequence-programmable RNA binding protein]-COOH. In other embodiments, the fusion protein comprises, consists of, or consists essentially of the structure NH₂-[guide nucleotide sequence-programmable RNA binding protein]-[linker]-[enzyme with cytidine deaminase activity]-COOH.

In some embodiments, the guide nucleotide sequence-programmable RNA binding protein is bound to a guide RNA (gRNA), a crisprRNA (crRNA), and/or a trans-activating crRNA (tracrRNA).

In some embodiments, the RMMP protein is encoded by a polynucleotide having a sequence comprising, consisting of, or consisting essentially of all or part of a sequence selected from NM_001080432, NM 019852, NM_020961, NM_024086, NM_001270531, NM 001270532, NM 001270533, NM 004906, NM_152857, NM 152858, NM_017758 and a sequence listed in the Additional Sequences section herein, and a biological equivalent of each thereof.

### Polynucleotides and Vectors

Provided herein are polynucleotides encoding a fusion protein comprising, consisting of, or consisting essentially of: (i) a guide nucleotide sequence-programmable RNA binding protein; and (ii) an effector enzyme. Exemplary effector enzymes include, without limitation, RMMPs and enzymes with cytidine deaminase activity.

In some aspects, provided herein are polynucleotides encoding a fusion protein comprising, consisting of, or consisting essentially of: (i) a guide nucleotide sequence-programmable RNA binding protein; and (ii) an RMMP protein. In some aspects, provided herein are polynucleotides encoding a fusion protein comprising, consisting of, or consisting essentially of: (i) a guide nucleotide sequence-programmable RNA binding protein; and (ii) an enzyme with cytidine deaminase activity (e.g., Apobec-1). In some embodiments, the polynucleotides further comprise a nucleic acid sequence encoding a linker peptide.

In some aspects, provided herein are vectors comprising, consisting of, or consisting essentially of a polynucleotide encoding a fusion protein comprising, consisting of, or consisting essentially of: (i) a guide nucleotide sequence-programmable RNA binding protein; and (ii) an RMMP protein. In some aspects, provided herein are vectors comprising, consisting of, or consisting essentially of a polynucleotide encoding a fusion protein comprising, consisting of, or consisting essentially of: (i) a guide nucleotide sequence-programmable RNA binding protein; and (ii) an enzyme with cytidine deaminase activity (e.g., Apobec-1). In some embodiments, the polynucleotides further comprise a nucleic acid sequence encoding a linker peptide.

In some embodiments, the vector is an adenoviral vector, an adeno-associated viral vector, or a lentiviral vector. In some embodiments, the vector further comprises one or more expression control elements operably linked to the polynucleotide. In some embodiments, the vector further comprises one or more selectable markers.

In some embodiments, the vector further comprises, consists of, or consists essentially of a polynucleotide encoding either (i) a gRNA, or (ii) a crRNA and a tracrRNA. In some embodiments, the gRNA or the crRNA comprises a nucleotide sequence complementary to a target RNA.

### Cells

Provided herein are cells comprising, consisting of, or consisting essentially of one or more vectors comprising, consisting of, or consisting essentially of a polynucleotide encoding a fusion protein comprising, consisting of, or consisting essentially of: (i) a guide nucleotide sequence-programmable RNA binding protein; and (ii) an effector enzyme. Exemplary effector enzymes include, without limitation, RMMPs and enzymes with cytidine deaminase activity.

In some aspects, provided herein are cells comprising, consisting of, or consisting essentially of one or more vectors comprising, consisting of, or consisting essentially of a polynucleotide encoding a fusion protein comprising, consisting of, or consisting essentially of: (i) a guide nucleotide sequence-programmable RNA binding protein; and (ii) an RMMP protein. In some aspects, provided herein are cells comprising, consisting of, or consisting essentially of one or more vectors comprising, consisting of, or consisting essentially of a polynucleotide encoding a fusion protein comprising, consisting of, or consisting essentially of: (i) a guide nucleotide sequence-programmable RNA binding protein; and (ii) an enzyme with cytidine deaminase activity (e.g., Apobec-1). In some embodiments, the polynucleotides further comprise a nucleic acid sequence encoding a linker peptide.

In some aspects, provided herein are cells comprising, consisting of, or consisting essentially of a fusion protein comprising, consisting of, or consisting essentially of: (i) a guide nucleotide sequence-programmable RNA binding protein; and (ii) an RMMP protein. In some aspects, provided herein are cells comprising, consisting of, or consisting essentially of a fusion protein comprising, consisting of, or consisting essentially of: (i) a guide nucleotide sequence-programmable RNA binding protein; and (ii) an enzyme with cytidine deaminase activity (e.g., Apobec-1).

In some embodiments, the cell is a eukaryotic cell. In other embodiments, the cell is a prokaryotic cell. In some embodiments, the cell is a mammalian cell. In some embodiments, the cell is a bovine, murine, feline, equine, porcine, canine, simian, or human cell. In particular embodiments, the cell is a human cell. In some embodiments, the cell is isolated from a subject.

### RNA-Targeted CRISPR Systems

Provided herein are systems for modulating RNA, the systems comprising, consisting of, or consisting essentially of: (i) fusion protein comprising, consisting of, or consisting essentially of: (a) a guide nucleotide sequence-programmable RNA binding protein; and (b) an effector enzyme; and either (ii) a gRNA or (iii) a crRNA and a tracrRNA, wherein the gRNA or the crRNA comprises a sequence complementary to a target mRNA. In some embodiments, the complementary sequence is a spacer sequence.

In some aspects, provided herein are systems for modulation of RNA methylation, the systems comprising, consisting of, or consisting essentially of: (i) fusion protein comprising, consisting of, or consisting essentially of: (a) a guide nucleotide sequence-programmable RNA binding protein; and (b) an RMMP protein; and either (ii) a gRNA or (iii) a crRNA and a tracrRNA, wherein the gRNA or the crRNA comprises a sequence complementary to a target mRNA. In some embodiments, the complementary sequence is a spacer sequence.

In some aspects, provided herein are systems for upregulating or increasing translation of a target mRNA, the systems comprising, consisting of, or consisting essentially of: (i) fusion protein comprising, consisting of, or consisting essentially of: (a) a guide nucleotide sequence-programmable RNA binding protein; and (b) an RMMP protein; and either (ii) a gRNA or (iii) a crRNA and a tracrRNA, wherein the gRNA or the crRNA comprises a sequence complementary to a target mRNA. In some embodiments, the complementary sequence is a spacer sequence.

In some aspects, provided herein are systems for downregulating or decreasing translation of a target mRNA, the systems comprising, consisting of, or consisting essentially of: (i) fusion protein comprising, consisting of, or consisting essentially of: (a) a guide nucleotide sequence-programmable RNA binding protein; and (b) an RMMP protein; and either (ii) a gRNA or (iii) a crRNA and a tracrRNA, wherein the gRNA or the crRNA comprises a sequence complementary to a target mRNA. In some embodiments, the complementary sequence is a spacer sequence.

In some embodiments, increasing or upregulating translation refers to an increase in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is increased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

In some embodiments, decreasing or downregulating translation refers to an decrease in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is decreased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

The amount of peptide translated can be determined by any method known in the art. Non-limiting examples of suitable methods of detection include Western blots, ELISAs, mass spectrometry, immunohistochemistry, immunofluorescence, and use of a reporter gene such as a fluorescence reporter gene.

In some aspects, provided herein are systems for directing cytidine to uridine conversion of RNA, the systems comprising, consisting of, or consisting essentially of: (i) fusion protein comprising, consisting of, or consisting essentially of: (a) a guide nucleotide sequence-programmable RNA binding protein; and (b) an enzyme that has cytidine deaminase activity; and either (ii) a gRNA or (iii) a crRNA and a tracrRNA, wherein the gRNA or the crRNA comprises a sequence complementary to a target mRNA. In some embodiments, the complementary sequence is a spacer sequence.

In some embodiments of the systems described herein, the target mRNA comprises a PAM sequence. In other embodiments, the target mRNA does not comprise a PAM sequence. In some embodiments, the system comprises a PAMmer oligonucleotide. In other embodiments, the system does not comprise a PAMmer oligonucleotide. In some embodiments, aberrant methylation of the target mRNA is associated with a disease or condition.

### Methods

Provided herein are methods for modulating a target RNA, the methods comprising contacting the target RNA with any of the fusion proteins provided herein, wherein the fusion protein includes a guide nucleotide sequence-programmable RNA binding protein which binds a gRNA or a crRNA that hybridizes to a region of the target RNA.

In some aspects, provided herein are methods for modulating m⁶A RNA methylation of a target RNA, the methods comprising contacting the target mRNA with a fusion protein that includes a guide nucleotide sequence-programmable RNA binding protein and an RMMP, wherein the guide nucleotide sequence-programmable RNA binding protein binds a gRNA or a crRNA that hybridizes to a region of the target RNA.

In some aspects, provided herein are methods for cytidine to uridine conversion in a target RNA, the methods comprising contacting the target mRNA with a fusion protein that includes a guide nucleotide sequence-programmable RNA binding protein and an enzyme with cytidine deaminase activity (e.g., Apobec-1), wherein the guide nucleotide sequence-programmable RNA binding protein binds a gRNA or a crRNA that hybridizes to a region of the target RNA.

In some aspects, provided herein are methods for modulating: embryonic stem cell maintenance and/or differentiation, nervous system development, circadian rhythm, heat shock response, meiotic progression, DNA ultraviolet (UV) damage response, or XIST mediated gene silencing, the methods comprising contacting a target mRNA with a fusion protein comprising, consisting of, or consisting essentially of: (i) a guide nucleotide sequence-programmable RNA binding protein; and (ii) a RNA methylation modification protein (RMMP), or an equivalent thereof, wherein the guide nucleotide sequence-programmable RNA binding protein binds a gRNA or a crRNA that hybridizes to a region of the target RNA. In some embodiments, the target mRNA comprises a PAM sequence or complement thereof. In some embodiments, the target mRNA does not comprise a PAM sequence or complement thereof. In some embodiments, the target mRNA is in a cell. In some embodiments, the cell is a eukaryotic cell. In some embodiments, the cell is a prokaryotic cell. In some embodiments, the eukaryotic cell is a mammalian cell, optionally a bovine, murine, feline, equine, porcine, canine, simian, or human cell. In some embodiments, the cell is in a subject.

In some aspects, provided herein are methods for treating a disease or condition associated with m⁶A RNA methylation of a target RNA in a subject in need thereof, the methods comprising administering a fusion protein, polynucleotide, vector, viral particle, and/or cell as described herein to the subject, thereby treating the disease or condition associated with m⁶A RNA methylation. In some embodiments, the disease or condition associated with m⁶A RNA methylation is selected from the group consisting of cancer, growth retardation, developmental delay, facial dysmorphism, Alzheimer's disease, diabetes, and major depressive disorder. In some embodiments, the subject is a human. In some embodiments, the methods further comprise administering to the subject: (i) a gRNA complementary to the target RNA, or (ii) a crRNA complementary to the target RNA and a tracrRNA. In some embodiments, the methods further comprise administering a PAMmer to the subject.

In some aspects, provided herein are methods for post-transcriptionally increasing or upregulating gene expression, the methods comprising, consisting of, or consisting essentially of contacting a target mRNA with a fusion protein comprising, consisting of, or consisting essentially of: (a) a guide nucleotide sequence-programmable RNA binding protein; and (b) an RMMP protein, wherein the guide nucleotide sequence-programmable RNA binding protein binds a gRNA or a crRNA that hybridizes to a region of the target RNA.

In some embodiments, increasing or upregulating gene expression refers to an increase in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is increased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

In some aspects, provided herein are methods for post-transcriptionally decreasing or downregulating gene expression, the methods comprising, consisting of, or consisting essentially of contacting a target mRNA with a fusion protein comprising, consisting of, or consisting essentially of: (a) a guide nucleotide sequence-programmable RNA binding protein; and (b) an RMMP protein, wherein the guide nucleotide sequence-programmable RNA binding protein binds a gRNA or a crRNA that hybridizes to a region of the target RNA.

In some embodiments, decreasing or downregulating gene expression refers to an decrease in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is decreased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

The amount of peptide translated can be determined by any method known in the art. Non-limiting examples of suitable methods of detection include Western blots, ELISAs, mass spectrometry, immunohistochemistry, immunofluorescence, and use of a reporter gene such as a fluorescence reporter gene.

In some embodiments of the methods described herein, the target mRNA comprises a PAM sequence. In other embodiments, the target mRNA does not comprise a PAM sequence. In some embodiments, the method further comprises providing a PAMmer oligonucleotide. In other embodiments, the method does not comprise providing a PAMmer oligonucleotide. In some embodiments, the target mRNA is in a cell. In some embodiments, the cell is a eukaryotic cell. In some embodiments, the cell is a prokaryotic cell. In some embodiments, the cell is a mammalian cell. In some embodiments, the cell is a bovine, murine, feline, equine, porcine, canine, simian, or human cell. In some embodiments, the cell is a plant cell. In some embodiments, the cell is in a subject.

In some aspects, also provided herein are methods for treating a disease or condition in a subject in need thereof, the methods comprising, consisting of, or consisting essentially of administering a fusion protein comprising, consisting of, or consisting essentially of: (a) a guide nucleotide sequence-programmable RNA binding protein; and (b) an RMMP protein, a polynucleotide encoding the fusion protein, a vector comprising the polynucleotide encoding the fusion protein, or viral particle comprising the vector to the subject, thereby decreasing or downregulating translation of a target mRNA in the subject. In some embodiments, aberrant methylation of the target mRNA is involved in the etiology of a disease or condition in the subject.

In some aspects, provided herein are methods for treating a disease or condition in a subject in need thereof, the methods comprising, consisting of, or consisting essentially of administering a fusion protein comprising, consisting of, or consisting essentially of: (a) a guide nucleotide sequence-programmable RNA binding protein; and (b) an enzyme with cytidine deaminase activity, a polynucleotide encoding the fusion protein, a vector comprising the polynucleotide encoding the fusion protein, or viral particle comprising the vector to the subject, thereby directing C-to-U conversions in a target RNA in the subject. In some embodiments, thymidine to cytidine (T>C) point mutations in the target RNA is involved in the etiology of a disease or condition in the subject.

In some embodiments of the methods described herein, the subject is a plant or an animal. In some embodiments, the subject is a mammal. In some embodiments, the mammal is a bovine, equine, porcine, canine, feline, simian, murine or human. In some embodiments, the subject is a human.

In some embodiments of the methods described herein, the subject is further administered (i) a gRNA complementary to the target mRNA, or (ii) a crRNA complementary to the target mRNA and a tracrRNA. In some embodiments, the complementary sequence is a spacer sequence.

### Cytidine to Uridine Conversion

Cytidine to uridine modification in RNA involves cytidine deaminase that deaminates a cytidine base into a uridine base. An example of C-to-U RNA editing involves the nuclear transcript encoding intestinal apolipoprotein B (apoB) (See, e.g., Anant et al., Curr. Opin. Lipidol. 12:159-165, 2001). Apo B100 is expressed in the liver and apo B48 is expressed in the intestines. In the intestines, the mRNA has a CAA sequence edited to be UAA, a stop codon, thus producing the shorter B48 form. ApoB RNA editing has important effects on lipoprotein metabolism, and defines distinct pathways for intestinal and hepatic lipid transport in mammals. ApoB RNA editing is mediated by a multicomponent complex with a minimal, two-component core composed of the catalytic deaminase apobec-1 and a competence factor, ACF. Apobec-1 functions as a dimer, with a composite active site representing asymmetric contributions from each monomer that permits both substrate binding and deamination, together with a leucine-rich pseudoactive site at the carboxyl terminus, involved in dimerization.

A second example of C-to-U RNA editing in mammals involves site-specific deamination of a CGA to UGA codon in the neurofibromatosis type 1 (NF1) mRNA (See, e.g., Skuse et al., Nucleic Acids Res. 24:478-485, 1996). NF1 RNA editing generates a translational termination codon at position 3916 that is predicted to truncate the protein product neurofibromin at the 5′ end of a critical domain involved in GTPase activation (See, e.g., Cichowski, Cell 104:593-604, 2001). C-to-U editing of NF1 mRNA has been shown to occur in tumors that express both the type II transcript and apobec-1 (See, e.g., Mukhopadhyay et al., Am. J. Hum. Genet. 70 (1):38-50, 2002). A further example involves NAT1, which is homologous to the translational repressor eIF4G, and undergoes C-to-U editing at multiple sites, with the creation of stop codons that in turn reduce protein abundance (See, e.g., Yamanaka et al., Genes Dev. 11:321-333, 1997).

In some embodiments, the present disclosure provides fusion proteins that include (i) a guide nucleotide sequence-programmable RNA binding protein; and (ii) an effector enzyme. The effector enzyme can be, e.g., an enzyme that has cytidine deaminase activity, and/or an enzyme that features cytidine deaminase active sites. The effector enzyme can also have RNA specificity and allows targeted nucleoside deamination of an RNA. The effector enzyme can be, e.g., an Apolipoprotein B mRNA editing enzyme, catalytic polypeptide 1 (Apobec-1). Apobec-1 related genes that feature cytidine deaminase active sites, including Apobec-2/ARCD1, activation-induced deaminase (AID), and phorbolins/ARCD2-7/apobec-3, are also contemplated (See, e.g., Blanc and Davidson, J Biol Chem, 278(3):1395-8, 2003). C-to-U editing can, for example, be used in transcript repair in diseases related to thymidine to cytidine (T>C) or adenosine to guanosine (A>G) point mutations (See, e.g., Vu and Tsukahara, Biosci Trends, 11(3):243-253, 2017).

### Viral Particles

Provided herein are viral particles comprising, consisting of, or consisting essentially of a vector comprising, consisting of, or consisting essentially of a polynucleotide encoding a fusion protein comprising, consisting of, or consisting essentially of: (i) a guide nucleotide sequence-programmable RNA binding protein; and (ii) an effector enzyme. Exemplary effector enzymes include, without limitation, RMMPs and enzymes with cytidine deaminase activity.

In some aspects, provided herein are viral particles comprising, consisting of, or consisting essentially of a vector comprising, consisting of, or consisting essentially of a polynucleotide encoding a fusion protein comprising, consisting of, or consisting essentially of: (i) a guide nucleotide sequence-programmable RNA binding protein; and (ii) an RMMP protein. In some embodiments, the polynucleotides further comprise a nucleic acid sequence encoding a linker peptide.

In some aspects, provided herein are viral particles comprising, consisting of, or consisting essentially of a vector comprising, consisting of, or consisting essentially of a polynucleotide encoding a fusion protein comprising, consisting of, or consisting essentially of: (i) a guide nucleotide sequence-programmable RNA binding protein; and (ii) an enzyme with cytidine deaminase activity (e.g., Apobec-1). In some embodiments, the polynucleotides further comprise a nucleic acid sequence encoding a linker peptide.

In general methods of packaging genetic material such as RNA or DNA into one or more vectors is well known in the art. For example, the genetic material may be packaged using a packaging vector and cell lines and introduced via traditional recombinant methods.

In some embodiments, the packaging vector may include, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter.

The retroviral packaging plasmid may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell. Similar vector based systems may employ other vectors such as sleeping beauty vectors or transposon elements.

The resulting packaged expression systems may then be introduced via an appropriate route of administration, discussed in detail with respect to the method aspects disclosed herein.

### Compositions

Also provided by this invention is a composition comprising any one or more of the fusion proteins and a carrier. In some embodiments, the carrier is a pharmaceutically acceptable carrier. In some embodiments, the composition is a pharmaceutical composition comprising one or more fusion proteins and a pharmaceutically acceptable carrier. In some embodiments, the composition or pharmaceutical composition further comprises one or more gRNAs, crRNAs, and/or tracrRNAs.

Briefly, pharmaceutical compositions of the present invention may comprise an fusion proteins or a polynucleotide encoding said fusion protein, optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

### Kits

Provided herein are kits comprising, consisting of, or consisting essentially of one or more fusion proteins, polynucleotides encoding a fusion protein, vectors comprising the polynucleotide, or viral particles comprising the vector, wherein the fusion protein comprises, consists of, or consists essentially of: (a) a guide nucleotide sequence-programmable RNA binding protein; and (b) an effector enzyme. Exemplary effector enzymes include, without limitation, RMMPs and enzymes with cytidine deaminase activity. In some embodiments, the kits further comprise, consist of, or consist essentially of instructions for use.

In some aspects, provided herein are kits comprising, consisting of, or consisting essentially of one or more fusion proteins, polynucleotides encoding a fusion protein, vectors comprising the polynucleotide, or viral particles comprising the vector, wherein the fusion protein comprises, consists of, or consists essentially of: (a) a guide nucleotide sequence-programmable RNA binding protein; and (b) an RMMP protein. In some embodiments, the kits further comprise, consist of, or consist essentially of instructions for use.

In some aspects, provided herein are kits comprising, consisting of, or consisting essentially of one or more fusion proteins, polynucleotides encoding a fusion protein, vectors comprising the polynucleotide, or viral particles comprising the vector, wherein the fusion protein comprises, consists of, or consists essentially of: (a) a guide nucleotide sequence-programmable RNA binding protein; and (b) an enzyme with cytidine deaminase activity (e.g., Apobec-1). In some embodiments, the kits further comprise, consist of, or consist essentially of instructions for use.

In some embodiments of the kits described herein, the kits further comprise, consist of, or consist essentially of one or more nucleic acids selected from: (i) a gRNA; (ii) a crRNA and a tracrRNA; (iii) a PAMmer oligonucleotide; and (iv) a vector for expressing the nucleic acid of (i), (ii), or (iii).

In some embodiments, the kits further comprise, consist of, or consist essentially of one or more reagents for carrying out a method of the disclosure. Non-limiting examples of such reagents comprise viral packaging cells, viral vectors, vector backbones, gRNAs, transfection reagents, transduction reagents, viral particles, and PCR primers.

### EXAMPLES

The invention is further described in the following examples, which do not limit the scope of the invention described in the claims.

### Example 1

A Cas directed m6A modification system was designed that (1) recognizes and edits a reporter mRNA construct in living cells at a base specific level, and (2) modulates m⁶A modification mediated silencing of expression from reporter transcripts in cell culture.

The minimal Cas-directed modification system of this example is composed of a nuclease-dead Cas (e.g. dCas9, dCas13) protein fused to the catalytic domain of the human METTL3, METTL14, METTL16, WTAP or FTO protein modules, a single guide RNA (sgRNA) driven by a U6 polymerase III promoter, and an optional inclusion of an antisense synthetic oligonucleotide composed alternating 2′OMe RNA and DNA bases (PAMmer). These are delivered to the nuclei of mammalian cells with transfection reagents that will together form a complex that may bind and modify mRNA after forming an RCas-RNA recognition complex. This allows for selective RNA modification in which targeted adenosine residues are methylated to m⁶A to be differentially recognized by the cellular machinery.

The catalytically active m6A modification module either consists of wildtype human METTL3, METTL14, METTL16, WTAP or FTO. These modules are fused to a semi-flexible XTEN peptide linker at its C or N-terminus, which is then fused to dCas9/13 at its C or N-terminus. To control for RNA-recognition independent background editing, fusion constructs lacking the dCas moiety have also been generated.

### Example 2

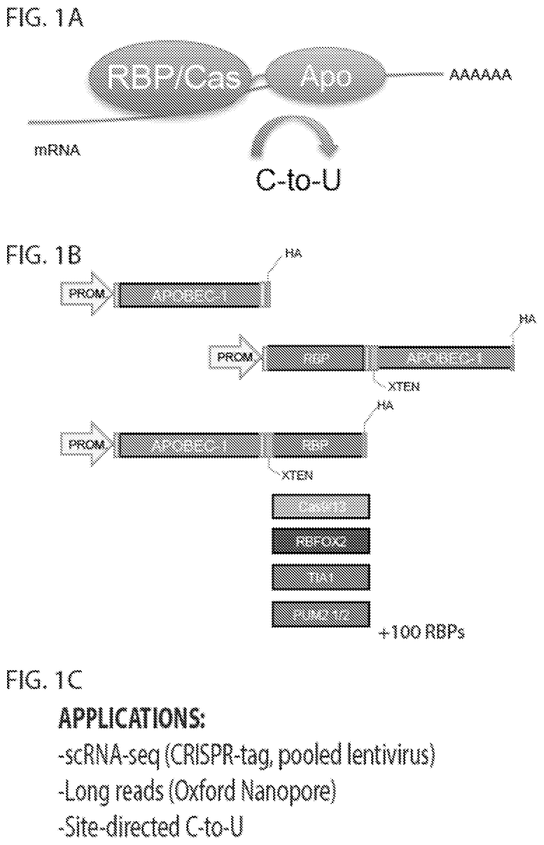

To carry out C-to-U editing of a target RNA, a Target RNA C-to-U Editing (TRACE) system was designed that is composed of an RNA-binding protein (RBP) or a RNA-targeting Cas module, fused to the rat cytidine deaminase enzyme APOBEC1 via an XTEN linker. Binding of this RBP-deaminase fusion protein to the target RNA thus allows binding-site proximal, specific C-to-U editing (Figure TA). Fusion proteins that include RNA-targeting dCas9, dCas13d, RBFOX2, TIA1, PUM2 1/2, and an additional 100 RBPs with published ENCODE eCLIP targets are cloned (FIG. 1B). The TRACE system can be used to identify RBP targets without the necessity for immunoprecipitation, thus allows for target identification from single cells (scRNA-seq) and long read direct RNA-sequencing (Oxford Nanopore). TRACE also allows for directed editing of a variety of disease (e.g., neurodegeneration, cancer)-causing RNA molecules (FIG. 1C).

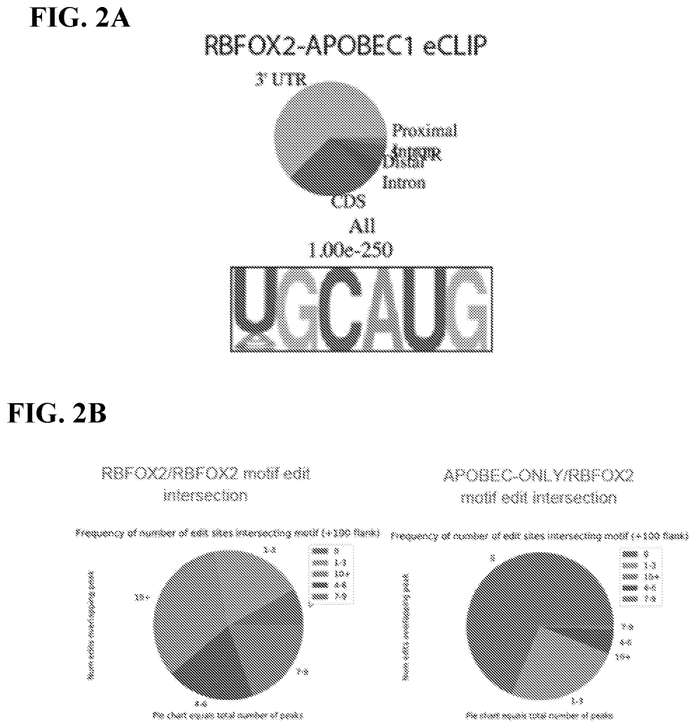

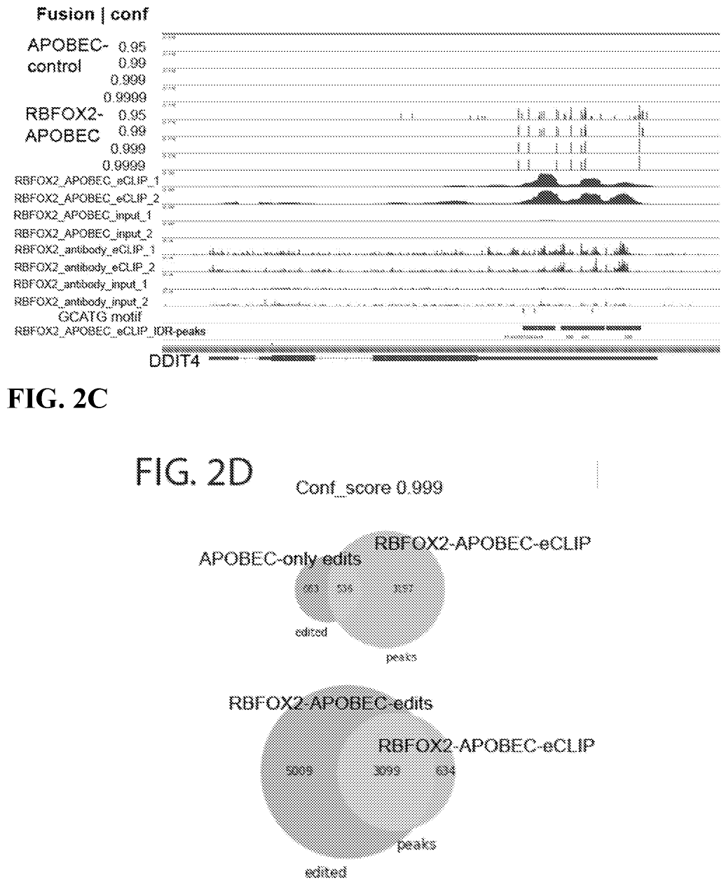

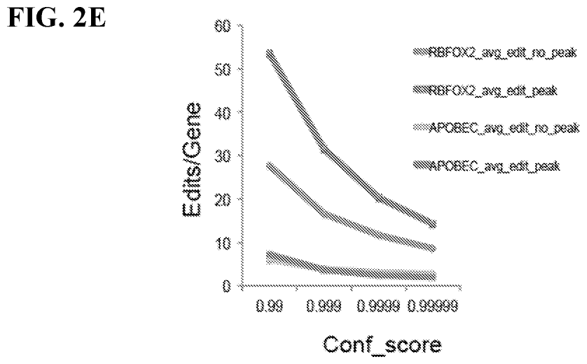

An RBFOX2-APOBEC1 fusion protein where RBFOX2 was fused to the rat cytidine deaminase enzyme APOBECT by an XTEN linker was generated. The fusion protein showed faithful binding to the binding motif of RBFOX2, GCAUG (FIG. 2A). As compared to C-to-U edits induced by APOBECT protein along, RBFOX2-APOBECT fusion protein resulted in C-to-U edits that were enriched at or within 100 bases of the RBFOX2 binding motifs (FIG. 2B). FIG. 2C shows binding of the RBFOX2-APOBECT fusion protein to target RNA DDIT4 and binding-site proximal, specific C-to-U editing directed by the fusion protein. The fusion protein directed C-to-U edits at or near the eCLIP binding sites for RBFOX2 (both fusion and endogenous RBFOX2 eCLIPs). The binding sites were discovered using eCLIP (See, e.g., Nostrand et al., Nature Methods 13: 508-514, 2016, which is incorporated herein by reference). The target specific C-to-U edits were not detected in the APOBEC-only overexpression control. As shown in FIG. 2D, significant RBFOX2-APOBEC directed C-to-U edits were detected on 83% of the RBFOX2 eCLIP targets, whereas only 14% of these targets show detectable edits from APOBECT overexpression alone. RBFOX2 targets showed a consistent 2-fold increase in total edits from RBFOX2-APOBECT when compared to non-eCLIP targets, and a 10-fold increase when compared to APOBEC1 control edits on the same target (FIG. 2E).

### EQUIVALENTS

It should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Xiao, M., et al., Functionality and substrate specificity of human box H/ACA guide RNAs. RNA, 2009. 15(1): p. 176-86.
2. Warda, A. S., et al., Human METTL16 is a N(6)-methyladenosine (m(6)A) methyltransferase that targets pre-mRNAs and various non-coding RNAs. EMBO Rep, 2017. 18(11): p. 2004-2014.
3. Jia, G., et al., N6-methyladenosine in nuclear RNA is a major substrate of the obesity-associated FTO. Nat Chem Biol, 2011. 7(12): p. 885-7.
4. Shi, H., et al., YTHDF3 facilitates translation and decay of N(6)-methyladenosine-modified RNA. Cell Res, 2017. 27(3): p. 315-328.
5. Xiao, W., et al., Nuclear m(6)A Reader YTHDC1 Regulates mRNA Splicing. Mol Cell, 2016. 61(4): p. 507-519.
6. Maity, A. and B. Das, N6-methyladenosine modification in mRNA: machinery, function and implications for health and diseases. FEBS J, 2016. 283(9): p. 1607-30.

ADDITIONAL SEQUENCES
METTL3

| | |
|---|---|
| source | 1..2038 |
| | /organism = ″Homo sapiens″ |
| | /mol_type = ″mRNA″ |
| | /db_xref = ″taxon:9606″ |
| | /chromosome = ″14″ |
| | /map = ″14q11.2″ |
| gene | 1..2038 |
| | /gene = ″METTL3″ |
| | /gene_synonym = ″hMETTL3; IME4; M6A; MT-A70; Spo8″ |
| | /note = ″methyltransferase like 3″ |
| | /db_xref = ″GeneID:56339″ |
| | /db_xref = ″HGNC:HGNC:17563″ |
| | /db_xref = ″MIM:612472″ |
| exon | 1..252 |
| | /gene = ″METTL3″ |
| | /gene_synonym = ″hMETTL3; IME4; M6A; MT-A70; Spo8″ |
| | /inference = ″alignment:Splign:2.1.0″ |
| misc_feature | 66..68 |
| | /gene = ″METTL3″ |
| | /gene_synonym = ″hMETTL3; IME4; M6A; MT-A70; Spo8″ |
| | /note = ″upstream in-frame stop codon″ |
| CDS | 153..1895 |
| | /gene = ″METTL3″ |
| | /gene_synonym = ″hMETTL3; IME4; M6A; MT-A70; Spo8″ |
| | /EC_number = ″2.1.1.62″ |
| | /note = ″adoMet-binding subunit of the human mRNA |
| | (N6-adenosine)-methyltransferase; mRNA m(6)A |
| | methyltransferase; N6-adenosine-methyltransferase 70 kDa |
| | subunit; methyltransferase-like protein 3; mRNA |
| | (2′-O-methyladenosine-N(6)-)-methyltransferase″ |
| | /codon_start = 1 |
| | /product = ″N6-adenosine-methyltransferase catalytic |
| | subunit″ |
| | /protein_id = ″NP_062826.2″ |
| | /db_xref = ″CCDS:CCDS32044.1″ |
| | /db_xref = ″GeneID:56339″ |
| | /db_xref = ″HGNC:HGNC:17563″ |
| | /db_xref = ″MIM:612472″ |

/translation = ″MSDTWSSIQAHKKQLDSLRERLQRRRKQDSGHLDLRNPEAALSP
TFRSDSPVPTAPTSGGPKPSTASAVPELATDPELEKKLLHHLSDLALTLPTDAVSICL
AISTPDAPATQDGVESLLQKFAAQELIEVKRGLLQDDAHPTLVTYADHSKLSAMMG
AV
AEKKGPGEVAGTVTGQKRRAEQDSTTVAAFASSLVSGLNSSASEPAKEPAKKSRKH
AA
SDVDLEIESLLNQQSTKEQQSKKVSQEILELLNTTTAKEQSIVEKFRSRGRAQVQEFC
DYGTKEECMKASDADRPCRKLHFRRIINKHTDESLGDCSFLNTCFHMDTCKYVHYEI
D
ACMDSEAPGSKDHTPSQELALTQSVGGDSSADRLFPPQWICCDIRYLDVSILGKFAV
V
MADPPWDIHMELPYGTLTDDEMRRLNIPVLQDDGFLFLWVTGRAMELGRECLNLW
GYE
RVDEIIWVKTNQLQRIIRTGRTGHWLNHGKEHCLVGVKGNPQGFNQGLDCDVIVAE
VR
STSHKPDEIYGMIERLSPGTRKIELFGRPHNVQPNWITLGNQLDGIHLLDPDVVARFK
QRYPDGIISKPKNL″

| | |
|---|---|
| misc_feature | 156..158 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″N-acetylserine, alternate. |
| | {ECO:0000244\|PubMed:19413330}; propagated from |
| | UniProtKB/Swiss-Prot (Q86U44.2); acetylation site″ |
| misc_feature | 156..158 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″Phosphoserine, alternate. |
| | {ECO:0000269\|PubMed:29348140}; propagated from |
| | UniProtKB/Swiss-Prot (Q86U44.2); phosphorylation site″ |
| misc_feature | 279..281 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″Phosphoserine. {ECO:0000244\|PubMed:16964243, |
| | ECO:0000244\|PubMed:18669648, ECO:0000244\|PubMed:20068231, |
| | ECO:0000244\|PubMed:23186163, ECO:0000269\|PubMed:29348140}; |
| | propagated from UniProtKB/Swiss-Prot (Q86U44.2); |
| | phosphorylation site″ |
| misc_feature | 294..296 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″Phosphoserine. {ECO:0000269\|PubMed:29348140}; |
| | propagated from UniProtKB/Swiss-Prot (Q86U44.2); |
| | phosphorylation site″ |
| misc_feature | 300..302 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″Phosphoserine. {ECO:0000269\|PubMed:29348140}; |
| | propagated from UniProtKB/Swiss-Prot (Q86U44.2); |
| | phosphorylation site″ |
| misc_feature | 780..797 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″propagated from UniProtKB/Swiss-Prot (Q86U44.2); |
| | Region: Nuclear localization signal. |
| | {ECO:0000269\|PubMed:29348140}″ |
| misc_feature | 807..809 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″Phosphoserine. {ECO:0000244\|PubMed:18669648, |
| | ECO:0000244\|PubMed:23186163, ECO:0000269\|PubMed:29348140}; |
| | propagated from UniProtKB/Swiss-Prot (Q86U44.2); |
| | phosphorylation site″ |
| misc_feature | 879..881 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″Phosphoserine. {ECO:0000244\|PubMed:18669648, |
| | ECO:0000269\|PubMed:29348140}; propagated from |
| | UniProtKB/Swiss-Prot (Q86U44.2); phosphorylation site″ |
| misc_feature | 1194..1196 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″Phosphothreonine. {ECO:0000244\|PubMed:23186163, |
| | ECO:0000269\|PubMed:29348140}; propagated from |
| | UniProtKB/Swiss-Prot (Q86U44.2); phosphorylation site″ |
| misc_feature | 1200..1202 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″Phosphoserine. {ECO:0000269\|PubMed:29348140}; |
| | propagated from UniProtKB/Swiss-Prot (Q86U44.2); |
| | phosphorylation site″ |
| misc_feature | 1281..1286 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″propagated from UniProtKB/Swiss-Prot (Q86U44.2); |
| | Region: S-adenosyl-L-methionine binding. |
| | {ECO:0000244\|PDB:5IL1, ECO:0000244\|PDB:51L2, |
| | ECO:0000244\|PDB:5K7U, ECO:0000244\|PDB:5K7W, |
| | ECO:0000244\|PDB:5L6D, ECO:0000244\|PDB:5L6E, |
| | ECO:0000269\|PubMed:27281194, ECO:0000269\|PubMed:27373337, |
| | ECO:0000269\|PubMed:27627798}″ |
| misc_feature | 1338..1382 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″propagated from UniProtKB/Swiss-Prot (Q86U44.2); |
| | Region: Gate loop 1. {ECO:0000303\|PubMed:27281194}″ |
| misc_feature | 1500..1514 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″propagated from UniProtKB/Swiss-Prot (Q86U44.2); |
| | Region: Interaction with METTL14. {ECO:0000244\|PDB:5IL0, |
| | ECO:0000244\|PDB:5IL1, ECO:0000244\|PDB:51L2, |
| | ECO:0000269\|PubMed:27281194}″ |
| misc_feature | 1536..1589 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″propagated from UniProtKB/Swiss-Prot (Q86U44.2); |
| | Region: Interphase loop. {ECO:0000303\|PubMed:27281194}″ |
| misc_feature | 1542..1592 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″propagated from UniProtKB/Swiss-Prot (Q86U44.2); |
| | Region: Interaction with METTL14. {ECO:0000244\|PDB:5IL0, |
| | ECO:0000244\|PDB:5IL1, ECO:0000244\|PDB:51L2, |
| | ECO:0000269\|PubMed:27281194}″ |
| misc_feature | 1545..1586 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″propagated from UniProtKB/Swiss-Prot (Q86U44.2); |
| | Region: Positively charged region required for |
| | RNA-binding. {ECO:0000269\|PubMed:27281194}″ |
| misc_feature | 1671..1697 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″propagated from UniProtKB/Swiss-Prot (Q86U44.2); |
| | Region: Gate loop 2. {ECO:0000303\|PubMed:27281194}″ |
| misc_feature | 1758..1769 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″propagated from UniProtKB/Swiss-Prot (Q86U44.2); |
| | Region: S-adenosyl-L-methionine binding. |
| | {ECO:0000244\|PDB:5IL1, ECO:0000244\|PDB:51L2, |
| | ECO:0000244\|PDB:5K7U, ECO:0000244\|PDB:5K7W, |
| | ECO:0000244\|PDB:5L6D, ECO:0000244\|PDB:5L6E, |
| | ECO:0000269\|PubMed:27281194, ECO:0000269\|PubMed:27373337, |
| | ECO:0000269\|PubMed:27627798}″ |
| misc_feature | 1797..1802 |
| | /gene = ″METTL3″ |
| | /gene_synonym = ″hMETTL3; IME4; M6A; MT-A70; Spo8″ |
| | /experiment = ″experimental evidence, no additional details |
| | recorded″ |
| | /note = ″propagated from UniProtKB/Swiss-Prot (Q86U44.2); |
| | Region: S-adenosyl-L-methionine binding. |
| | {ECO:0000244\|PDB:5IL1, ECO:0000244\|PDB:51L2, |
| | ECO:0000244\|PDB:5K7U, ECO:0000244\|PDB:5K7W, |
| | ECO:0000244\|PDB:5L6D, ECO:0000244\|PDB:5L6E, |
| | ECO:0000269\|PubMed:27281194, ECO:0000269\|PubMed:27373337, |
| | ECO:0000269\|PubMed:27627798}″ |
| exon | 253..470 |
| | /gene = ″METTL3″ |
| | /gene_synonym = ″hMETTL3; IME4; M6A; MT-A70; Spo8″ |
| | /inference = ″alignment:Splign:2.1.0″ |
| exon | 471..875 |
| | /gene = ″METTL3″ |
| | /gene_synonym = ″hMETTL3; IME4; M6A; MT-A70; Spo8″ |
| | /inference = ″alignment:Splign:2.1.0″ |
| exon | 876..1051 |
| | /gene = ″METTL3″ |
| | /gene_synonym = ″hMETTL3; IME4; M6A; MT-A70; Spo8″ |
| | /inference = ″alignment:Splign:2.1.0″ |
| exon | 1052..1268 |
| | /gene = ″METTL3″ |
| | /gene_synonym = ″hMETTL3; IME4; M6A; MT-A70; Spo8″ |
| | /inference = ″alignment:Splign:2.1.0″ |
| exon | 1269..1456 |
| | /gene = ″METTL3″ |
| | /gene_synonym = ″hMETTL3; IME4; M6A; MT-A70; Spo8″ |
| | /inference = ″alignment:Splign:2.1.0″ |
| exon | 1457..1495 |
| | /gene = ″METTL3″ |
| | /gene_synonym = ″hMETTL3; IME4; M6A; MT-A70; Spo8″ |
| | /inference = ″alignment:Splign:2.1.0″ |
| exon | 1496..1604 |
| | /gene = ″METTL3″ |
| | /gene_synonym = ″hMETTL3; IME4; M6A; MT-A70; Spo8″ |
| | /inference = ″alignment:Splign:2.1.0″ |
| exon | 1605..1670 |
| | /gene = ″METTL3″ |
| | /gene_synonym = ″hMETTL3; IME4; M6A; MT-A70; Spo8″ |
| | /inference = ″alignment:Splign:2.1.0″ |
| exon | 1671..1783 |
| | /gene = ″METTL3″ |
| | /gene_synonym = ″hMETTL3; IME4; M6A; MT-A70; Spo8″ |
| | /inference = ″alignment:Splign:2.1.0″ |
| exon | 1784..2022 |
| | /gene = ″METTL3″ |
| | /gene_synonym = ″hMETTL3; IME4; M6A; MT-A70; Spo8″ |
| | /inference = ″alignment:Splign:2.1.0″ |
| regulatory | 1990..1995 |
| | /regulatory class = ″polyA_signal_sequence″ |
| | /gene = ″METTL3″ |
| | /gene_synonym = ″hMETTL3; IME4; M6A; MT-A70; Spo8″ |
| polyA_site | 2022 |
| | /gene = ″METTL3″ |
| | /gene_synonym = ″hMETTL3; IME4; M6A; MT-A70; Spo8″ |

cDNA:
aaatgacttttctgtcttgctcagctccaggggtcattttccggttagccttcggggtgtccgcgtgagaattggctatatcctggagcgag
tgctgggaggtgctagtccgccgcgccttattcgagaggtgtcagggctgggagactaggatgtcggacacgtggagctctatccag
gcccacaagaagcagctggactctctgcgggagaggctgcagcggaggcggaagcaggactcggggcacttggatctacggaat
ccagaggcagcattgtctccaaccttccgtagtgacagcccagtgcctactgcacccacctctggtggccctaagcccagcacagctt
cagcagttcctgaattagctacagatcctgagttagagaagaagttgctacaccacctctctgatctggccttaacattgcccactgatgc
tgtgtccatctgtcttgccatctccacgccagatgctcctgccactcaagatggggtagaaagcctcctgcagaagtttgcagctcagga
gttgattgaggtaaagcgaggtctcctacaagatgatgcacatcctactcttgtaacctatgctgaccattccaagctctctgccatgatg
ggtgctgtggcagaaaagaagggccctggggaggtagcagggactgtcacagggcagaagcggcgtgcagaacaggactcgact
acagtagctgcctttgccagttcgttagtctctggtctgaactcttcagcatcggaaccagcaaaggagccagccaagaaatcaaggaa
acatgctgcctcagatgttgatctggagatagagagccttctgaaccaacagtccactaaggaacaacagagcaagaaggtcagtca
ggagatcctagagctattaaatactacaacagccaaggaacaatccattgttgaaaaatttcgctctcgaggtcgggcccaagtgcaag
aattctgtgactatggaaccaaggaggagtgcatgaaagccagtgatgctgatcgaccctgtcgcaagctgcacttcagacgaattatc
aataaacacactgatgagtctttaggtgactgctctttccttaatacatgtttccacatggatacctgcaagtatgttcactatgaaattgatg
cttgcatggattctgaggcccctggcagcaaagaccacacgccaagccaggagcttgctcttacacagagtgtcggaggtgattcca
gtgcagaccgactcttcccacctcagtggatctgttgtgatatccgctacctggacgtcagtatcttgggcaagtttgcagttgtgatggct
gacccaccctgggatattcacatggaactgccctatgggaccctgacagatgatgagatgcgcaggctcaacatacccgtactacag
gatgatggctttctcttcctctgggtcacaggcagggccatggagttggggagagaatgtctaaacctctgggggtatgaacgggtag
atgaaattatttgggtgaagacaaatcaactgcaacgcatcattcggacaggccgtacaggtcactggttgaaccatgggaaggaaca
ctgcttggttggtgtcaaaggaaatccccaaggcttcaaccagggtctggattgtgatgtgatcgtagctgaggttcgttccaccagtcat
aaaccagatgaaatctatggcatgattgaaagactatctcctggcactcgcaagattgagttatttggacgaccacacaatgtgcaaccc
aactggatcacccttggaaaccaactggatgggatccacctactagacccagatgtggttgcacggttcaagcaaaggtacccagatg
gtatcatctctaaacctaagaatttatagaagcacttccttacagagctaagaatccatagccatggctctgtaagctaaacctgaagagt
gatatttgtacaatagctttcttctttatttaaataaacatttgtattgtagttgggattctgaaaaaaaaaaaaaaaaaa
METTL14

| FEATURES | Location/Qualifiers |
|---|---|
| source | 1..3520 |
| | /organism = ″Homo sapiens″ |
| | /mol_type = ″mRNA″ |
| | /db_xref = ″taxon:9606″ |
| | /chromosome = ″4″ |
| | /map = ″4q26″ |
| gene | 1..3520 |
| | /gene = ″METTL14″ |
| | /gene_synonym = ″hMETTL14″ |
| | /note = ″methyltransferase like 14″ |
| | /db_xref = ″GeneID:57721″ |
| | /db_xref = ″HGNC:HGNC:29330″ |
| | /db_xref = ″MIM:616504″ |
| exon | 1..231 |
| | /gene = ″METTL14″ |
| | /gene_synonym = ″hMETTL14″ |
| | /inference = ″alignment:Splign:2.1.0″ |
| misc_feature | 127..129 |
| | /gene = ″METTL14″ |
| | /gene_synonym = ″hMETTL14″ |
| | /note = ″upstream in-frame stop codon″ |
| CDS | 166..1536 |
| | /gene = ″METTL14″ |
| | /gene_synonym = ″hMETTL14″ |
| | /EC_number = ″2.1.1.62″ |
| | /note = ″methyltransferase-like protein 14; |
| | N6-adenosine-methyltransferase subunit METTL14″ |
| | /codon_start = 1 |
| | /product = ″N6-adenosine-methyltransferase non-catalytic |
| | subunit″ |
| | /protein_id = ″NP_066012.1″ |
| | /db_xref = ″CCDS:CCDS34053.1″ |
| | /db_xref = ″GeneID:57721″ |
| | /db_xref = ″HGNC:HGNC:29330″ |
| | /db_xref = ″MIM:616504″ |

/translation = ″MDSRLQEIRERQKLRRQLLAQQLGAESADSIGAVLNSKDEQREI
AETRETCRASYDTSAPNAKRKYLDEGETDEDKMEEYKDELEMQQDEENLPYEEEIY
KD
SSTFLKGTQSLNPHNDYCQHFVDTGHRPQNFIRDVGLADRFEEYPKLRELIRLKDELI
AKSNTPPMYLQADIEAFDIRELTPKFDVILLEPPLEEYYRETGITANEKCWTWDDIMK
LEIDEIAAPRSFIFLWCGSGEGLDLGRVCLRKWGYRRCEDICWIKTNKNNPGKTKTL
D
PKAVFQRTKEHCLMGIKGTVKRSTDGDFIHANVDIDLIITEEPEIGNIEKPVEIFHII
EHFCLGRRRLHLFGRDSTIRPGWLTVGPTLTNSNYNAETYASYFSAPNSYLTGCTEEI
ERLRPKSPPPKSKSDRGGGAPRGGGRGGTSAGRGRERNRSNFRGERGGFRGGRGGA
HR
GGFPPR″

| | |
|---|---|
| misc_feature | 568..573 |
| | /gene = ″METTL14″ |
| | /gene_synonym = ″hMETTL14″ |
| | /experiment = ″experimental evidence, no additional details |
| | recorded″ |
| | /note = ″propagated from UniProtKB/Swiss-Prot (Q9HCE5.2); |
| | Region: Interaction with METTL3. {ECO:0000244\|PDB:51L0, |
| | ECO:0000244\|PDB:51L1, ECO:0000244\|PDB:51L2, |
| | ECO:0000269\|PubMed:27281194}″ |
| misc_feature | 874..879 |
| | /gene = ″METTL14″ |
| | /gene_synonym = ″hMETTL14″ |
| | /experiment = ″experimental evidence, no additional details |
| | recorded″ |
| | /note = ″propagated from UniProtKB/Swiss-Prot (Q9HCE5.2); |
| | Region: Interaction with METTL3. {ECO:0000244\|PDB:51L0, |
| | ECO:0000244\|PDB:5IL1, ECO:0000244\|PDB:51L2, |
| | ECO:0000269\|PubMed:27281194}″ |
| misc_feature | 898..927 |
| | /gene = ″METTL14″ |
| | /gene_synonym = ″hMETTL14″ |
| | /experiment = ″experimental evidence, no additional details |
| | recorded″ |
| | /note = ″propagated from UniProtKB/Swiss-Prot (Q9HCE5.2); |
| | Region: Positively charged region required for |
| | RNA-binding. {ECO:0000269\|PubMed:27281194}″ |
| misc_feature | 928..939 |
| | /gene = ″METTL14″ |
| | /gene_synonym = ″hMETTL14″ |
| | /experiment = ″experimental evidence, no additional details |
| | recorded″ |
| | /note = ″propagated from UniProtKB/Swiss-Prot (Q9HCE5.2); |
| | Region: Interaction with METTL3. {ECO:0000244\|PDB:51L0, |
| | ECO:0000244\|PDB:5IL1, ECO:0000244\|PDB:51L2, |
| | ECO:0000269\|PubMed:27281194}″ |
| misc_feature | 997..1026 |
| | /gene = ″METTL14″ |
| | /gene_synonym = ″hMETTL14″ |
| | /experiment = ″experimental evidence, no additional details |
| | recorded″ |
| | /note = ″propagated from UniProtKB/Swiss-Prot (Q9HCE5.2); |
| | Region: Interaction with METTL3. {ECO:0000244\|PDB:5IL0, |
| | ECO:0000244\|PDB:5IL1, ECO:0000244\|PDB:51L2, |
| | ECO:0000269\|PubMed:27281194}″ |
| misc_feature | 1054..1059 |
| | /gene = ″METTL14″ |
| | /gene_synonym = ″hMETTL14″ |
| | /experiment = ″experimental evidence, no additional details |
| | recorded″ |
| | /note = ″propagated from UniProtKB/Swiss-Prot (Q9HCE5.2); |
| | Region: Positively charged region required for |
| | RNA-binding. {ECO:0000269\|PubMed:27281194}″ |
| misc_feature | 1087..1101 |
| | /gene = ″METTL14″ |
| | /gene_synonym = ″hMETTL14″ |
| | /experiment = ″experimental evidence, no additional details |
| | recorded″ |
| | /note = ″propagated from UniProtKB/Swiss-Prot (Q9HCE5.2); |
| | Region: Interaction with METTL3. {ECO:0000244\|PDB:5IL0, |
| | ECO:0000244\|PDB:5IL1, ECO:0000244\|PDB:51L2, |
| | ECO:0000269\|PubMed:27281194}″ |
| misc_feature | 1360..1362 |
| | /gene = ″METTL14″ |
| | /gene_synonym = ″hMETTL14″ |
| | /experiment = ″experimental evidence, no additional details |
| | recorded″ |
| | /note = ″Phosphoserine. {ECO:0000244\|PDB:5IL0, |
| | ECO:0000244\|PDB:5IL1 ECO:0000244\|PDB:51L2, |
| | ECO:0000244\|PubMed:24275569, ECO:0000269\|PubMed:27281194, |
| | ECO:0000269\|PubMed:29348140}; propagated from |
| | UniProtKB/Swiss-Prot (Q9HCE5.2); phosphorylation site″ |
| exon | 232..320 |
| | /gene = ″METTL14″ |
| | /gene_synonym = ″hMETTL14″ |
| | /inference = ″alignment:Splign:2.1.0″ |
| exon | 321..408 |
| | /gene = ″METTL14″ |
| | /gene_synonym = ″hMETTL14″ |
| | /inference = ″alignment:Splign:2.1.0″ |
| exon | 409..489 |
| | /gene = ″METTL14″ |
| | /gene_synonym = ″hMETTL14″ |
| | /inference = ″alignment:Splign:2.1.0″ |
| exon | 490..577 |
| | /gene = ″METTL14″ |
| | /gene_synonym = ″hMETTL14″ |
| | /inference = ″alignment:Splign:2.1.0″ |
| exon | 578..668 |
| | /gene = ″METTL14″ |
| | /gene_synonym = ″hMETTL14″ |
| | /inference = ″alignment:Splign:2.1.0″ |
| exon | 669..810 |
| | /gene = ″METTL14″ |
| | /gene_synonym = ″hMETTL14″ |
| | /inference = ″alignment:Splign:2.1.0″ |
| exon | 811..903 |
| | /gene = ″METTL14″ |
| | /gene_synonym = ″hMETTL14″ |
| | /inference = ″alignment:Splign:2.1.0″ |
| exon | 904..1020 |
| | /gene = ″METTL14″ |
| | /gene_synonym = ″hMETTL14″ |
| | /inference = ″alignment:Splign:2.1.0″ |
| exon | 1021..1231 |
| | /gene = ″METTL14″ |
| | /gene_synonym = ″hMETTL14″ |
| | /inference = ″alignment:Splign:2.1.0″ |
| exon | 1232..3504 |
| | /gene = ″METTL14″ |
| | /gene_synonym = ″hMETTL14″ |
| | /inference = ″alignment:Splign:2.1.0″ |

cDNA
gagccaattccggccgcgccggaagtctctactgaggaaagctatgaggatactctgttcgtaagctcccggtgaattttgttccacag
actcggaagaaaggttggataagagttcactggagattgacaagtactcgggatagtgaaaagccggagttggaacatggatagccg
cttgcaggagatccgggagcggcagaagttacggcgacagctcctcgcgcagcagttgggagctgaaagtgccgacagcattggt
gccgtgttaaatagcaaagatgagcagagagaaattgctgaaacaagagaaacttgcagggcttcctatgatacctctgctccaaatgc
aaaacgtaagtatctggatgaaggagagacagatgaagacaaaatggaagaatataaggatgaactagaaatgcaacaggatgaag
aaaatttgccatatgaagaagagatttacaaagattctagtacttttcttaagggaacacagagcttaaatccccataatgattactgccaa
cattttgtagacactggacatagacctcagaatttcatcagggatgtaggtttagctgacagatttgaagaatatcctaaactgagggagc
tcatcaggctaaaggatgagttaatagctaaatctaacactcctcccatgtacttacaagccgatatagaagcctttgacatcagagaact
aacacccaaatttgatgtgattcttctggaaccccctttagaagaatattacagagaaactggcatcactgctaatgaaaaatgctggactt
gggatgatattatgaagttagaaattgatgagattgcagcacctcgatcatttatttttctctggtgtggttctggggaggggttggaccttg
gaagagtgtgtttacgaaaatggggttacagaagatgtgaagatatttgttggattaaaaccaataaaaacaatcctgggaagactaaga
ctttagatccaaaggctgtctttcagagaacaaaggaacactgcctcatggggatcaaaggaactgtgaagcgtagcacagacgggg
acttcattcatgctaatgttgacattgacttaattatcacagaagaacctgaaattggcaatatagaaaaacctgtagaaatttttcatataatt
gagcatttttgtcttggtagaagacgccttcatctatttggaagagatagtacaattcgaccaggctggctcacagttggaccaacgctta
caaatagcaactacaatgcagaaacatatgcatcctatttcagtgctcctaattcctacttgactggttgtacagaagaaattgagagactt
cgaccaaaatcgcctcctcccaaatctaaatctgaccgaggaggtggagctcccagaggtggaggaagaggtggaacttctgctggc
cgtggacgagaaagaaatagatctaacttccgaggagaaagaggtggctttagagggggccgtggaggagcacacagaggtggct
ttccacctcgataattgttgaagacattgaacctattcatcctcctctaaccttctttattgtaattaaatttcaagtgggagacttaactttaga
actcacttccagcttgcactttgctttaatttctctgagctgcaagaatgtcttagcgagccttgcttgcagttgtcacacacactgtctggttt
ttttcaggataaatgaatgattctgccttttgttatgtgcgtgaacagaatggaacaactcaagtagcttcatcttcagagactgaatttattct
gatagacttcagctaattacaaaggattttgctaatttttgggaataaataatggaaaaagatccagtctgtggtatcatgctagtgctgaca
gggccttgatagaatagagttggaaaagatggtaagcttttgtcagggttttaacattttcttgatgaaacaataaaaagaggtaagcttttt
tcttctttttttttaagttttaaataaactcagatataatttgaatactgaagaaattaagagactttgaacaaaaactcttcccaaatctaaatt
tgataggggaggtggagattccaggggtgggtgaaagaagagatagaacttagcaggcagacttaaaaaaaaaaaaaaagtttatcat
cataatctcaattttgtggctatgactcctaatcacgcttcctaagaagcaaaggaggacaaatattcatgtgctagatagcactgtggtgt
ggacttgaacttggattgaccttaaattttatattcctcaaataaaagagaggcagcgacaagatacctcattatcagatgcttggtttatac
attttgggactaaaatacttggtgatgaaatgacatacacctttaaacttgttatggagatagtttaatgtaaaaccaactacggaaaaccct
caacttaaggatacagcttggaaattggaactgcaattgccttttattaaaaccatatggtgtgatgtttgtttttaaaattatataagactttat
gctgtcacttctcttgctgtactgtaattcatgttttaaatgaatttgataatgaaattatactattatcattcttgatgaatacttttcttattt
ttatgatttttctaatgaaactttaaacttttgagatttgagagtctgttttctataagtagaattactgttgttacaaaatgaaaaaggactgac
ctaaaatcagtctcttcttttggtctgtgatggattttaatggccgttctgtgctcatatatacctaagatgagattatattacatccaccaaaga
ctcagtttgaagataaggaatgagtgatagaagaaataaggctgagatccttaaaagcctaattaatttaactcgcttaacccattagtactatct
agtacaagacccctttttttttgctgaaattatggtatattttcaacttcactaattacaaattatctagatttagaactctatatgtcagcattg
acctgggaatgaagtcaggatagagaaattccacttgcctgtgatgggtccttagaagtatcagctaaggagtgaccctgtcctatacaca
gggctctctattacgttccataccctgggcctacccaaggtgacattcctgctgtttacatggcataggcacctgtgagatcagtgtcaca
atttcatcttagaaagaggtaggtatggctgctttgtcggttgaaagttaaggggagccatgatctaccatatttaggaaaaagttatttaaa
aaagagcagatggtggaaaaagaatgtaagacccagaatttatccctttgacaatgaatctggcctttttaatagcaggatggaattgatt
cactagtttttgctaactttcactttcagtaaaggttgaggtgttgtttttgcaatgactgtgtattcattgaggaaaggtttccaatgaaatttc
attactctgaaaaaaaaaaaaaaaaa//
METTL16

| FEATURES | Location/Qualifiers |
|---|---|
| source | 1..5758 |
| | /organism = ″Homo sapiens″ |
| | /mol_type = ″mRNA″ |
| | /db_xref = ″taxon:9606″ |
| | /chromosome = ″17″ |
| | /map = ″17p13.3″ |
| gene | 1..5758 |
| | /gene = ″METTL16″ |
| | /gene_synonym = ″METT10D″ |
| | /note = ″methyltransferase like 16″ |
| | /db_xref = ″GeneID:79066″ |
| | /db_xref = ″HGNC:HGNC:28484″ |
| exon | 1..148 |
| | /gene = ″METTL16″ |
| | /gene_synonym = ″METT10D″ |
| | /inference = ″alignment:Splign:2.1.0″ |
| misc_feature | 92..94 |
| | /gene = ″METTL16″ |
| | /gene_synonym = ″METT10D″ |
| | /note = ″upstream in-frame stop codon″ |
| CDS | 149..1837 |
| | /gene = ″METTL16″ |
| | /gene_synonym = ″METT10D″ |
| | /EC_number = ″2.1.1.62″ |
| | /EC_number = ″2.1.1.346″ |
| | /note = ″methyltransferase 10 domain containing; putative |
| | methyltransferase METT10D; methyltransferase-like protein |
| | 16; methyltransferase 10 domain-containing protein; |
| | N6-adenosine-methyltransferase METTL16; U6 snRNA |
| | methyltransferase″ |
| | /codon_start = 1 |
| | /product = ″U6 small nuclear RNA |
| | (adenine-(43)-N(6))-methyltransferase″ |
| | /protein_id = ″NP_076991.3″ |
| | /db_xref = ″CCDS:CCDS42232.1″ |
| | /db_xref = ″GeneID:79066″ |
| | /db_xref = ″HGNC:HGNC:28484″ |

/translation = ″MALSKSMHARNRYKDKPPDFAYLASKYPDFKQHVQINLNGRVSL
NFKDPEAVRALTCTLLREDFGLSIDIPLERLIPTVPLRLNYIHWVEDLIGHQDSDKST
LRRGIDIGTGASCIYPLLGATLNGWYFLATEVDDMCFNYAKKNVEQNNLSDLIKVV
KV
PQKTLLMDALKEESEHYDFCMCNPPFFANQLEAKGVNSRNPRRPPPSSVNTGGITEI
MAEGGELEFVKRIIHDSLQLKKRLRWYSCMLGKKCSLAPLKEELRIQGVPKVTYTEF
C
QGRTMRWALAWSFYDDVTVPSPPSKRRKLEKPRKPITFVVLASVMKELSLKASPLRS
E
TAEGIVVVTTWIEKILTDLKVQHKRVPCGKEEVSLFLTAIENSWIHLRRKKRERVRQL
REVPRAPEDVIQALEEKKPTPKESGNSQELARGPQERTPCGPALREGEAAAVEGPCPS
QESLSQEENPEPTEDERSEEKGGVEVLESCQGSSNGAQDQEASEQFGSPVAERGKRL
P
GVAGQYLFKCLINVKKEVDDALVEMHWVEGQNRDLMNQLCTYIRNQIFRLVAVN″

| | |
|---|---|
| misc_feature | 1013..1348 |
| | /gene = ″METTL16″ |
| | /gene_synonym = ″METT10D″ |
| | /experiment = ″experimental evidence, no additional details |
| | recorded″ |
| | /note = ″propagated from UniProtKB/Swiss-Prot (Q86W50.2); |
| | Region: VCR 1. {ECO:0000269\|PubMed:28525753}″ |
| misc_feature | 1133..1135 |
| | /gene = ″METTL16″ |
| | /gene_synonym = ″METT10D″ |
| | /experiment = ″experimental evidence, no additional details |
| | recorded″ |
| | /note = ″Phosphoserine. {ECO:0000244\|PubMed:18669648, |
| | ECO:0000244\|PubMed:18691976, ECO:0000244\|PubMed:23186163}; |
| | propagated from UniProtKB/Swiss-Prot (Q86W50.2); |
| | phosphorylation site″ |
| misc_feature | 1535..1537 |
| | /gene = ″METTL16″ |
| | /gene_synonym = ″METT10D″ |
| | /experiment = ″experimental evidence, no additional details |
| | recorded″ |
| | /note = ″Phosphothreonine. {ECO:0000250\|UniProtKB:Q9CQG2}; |
| | propagated from UniProtKB/Swiss-Prot (Q86W50.2); |
| | phosphorylation site″ |
| misc_feature | 1688..1834 |
| | /gene = ″METTL16″ |
| | /gene_synonym = ″METT10D″ |
| | /experiment = ″experimental evidence, no additional details |
| | recorded″ |
| | /note = ″propagated from UniProtKB/Swiss-Prot (Q86W50.2); |
| | Region: VCR 2. {ECO:0000269\|PubMed:28525753}″ |
| exon | 149..276 |
| | /gene = ″METTL16″ |
| | /gene_synonym = ″METT10D″ |
| | /inference = ″alignment:Splign:2.1.0″ |
| exon | 277..476 |
| | /gene = ″METTL16″ |
| | /gene_synonym = ″METT10D″ |
| | /inference = ″alignment:Splign:2.1.0″ |
| exon | 477..617 |
| | /gene = ″METTL16″ |
| | /gene_synonym = ″METT10D″ |
| | /inference = ″alignment:Splign:2.1.0″ |
| exon | 618..733 |
| | /gene = ″METTL16″ |
| | /gene_synonym = ″METT10D″ |
| | /inference = ″alignment:Splign:2.1.0″ |
| exon | 734..876 |
| | /gene = ″METTL16″ |
| | /gene_synonym = ″METT10D″ |
| | /inference = ″alignment:Splign:2.1.0″ |
| exon | 877..946 |
| | /gene = ″METTL16″ |
| | /gene_synonym = ″METT10D″ |
| | /inference = ″alignment:Splign:2.1.0″ |
| exon | 947..1036 |
| | /gene = ″METTL16″ |
| | /gene_synonym = ″METT10D″ |
| | /inference = ″alignment:Splign:2.1.0″ |
| exon | 1037..1210 |
| | /gene = ″METTL16″ |
| | /gene_synonym = ″METT1OD″ |
| | /inference = ″alignment:Splign:2.1.0″ |
| exon | 1211..5758 |
| | /gene = ″METTL16″ |
| | /gene_synonym = ″METT10D″ |
| | /inference = ″alignment:Splign:2.1.0″ |
| STS | 3505..3721 |
| | /gene = ″METTL16″ |
| | /gene_synonym = ″METT10D″ |
| | /standard_name = ″G54860″ |
| | /db_xref = ″UniSTS:163631″ |
| STS | 4552..4640 |
| | /gene = ″METTL16″ |
| | /gene_synonym = ″METT10D″ |
| | /standard_name = ″D8S2279″ |
| | /db_xref = ″UniSTS:473907″ |
| STS | 5445..5688 |
| | /gene = ″METTL16″ |
| | /gene_synonym = ″METT10D″ |
| | /standard_name = ″D17S1413E″ |
| | /db_xref = ″UniSTS:150458″ |
| STS | 5511..5640 |
| | /gene = ″METTL16″ |
| | /gene_synonym = ″METT10D″ |
| | /standard_name = ″D17S1430E″ |
| | /db_xref = ″UniSTS:150468″ |
| STS | 5578..5683 |
| | /gene = ″METTL16″ |
| | /gene_synonym = ″METT10D″ |
| | /standard_name = ″D17S1478E″ |
| | /db_xref = ″UniSTS:151684″ |
| STS | 5601..5698 |
| | /gene = ″METTL16″ |
| | /gene_synonym = ″METT10D″ |
| | /standard_name = ″WI-13902″ |
| | /db_xref = ″UniSTS:27351″ |

cDNA
acgaggctagatggcttcacaagatggcggcgcgctgggagcgtatcatctgcgtttctaggagcttcgctatgcggctgctttaagatt
ctagggttgtacaggcccacgccagacacgacgtctggcaggaacctcggcctcagagatggctctgagtaaatcaatgcatgcaag
aaatagatacaaggacaaacctcctgactttgcatatctggcatccaaatatccagattttaagcagcatgttcagataaatctgaatgga
agagtgagccttaattttaaagaccccgaagcagtcagagctctgacgtgtactctcctaagggaagattttggactttctattgatattcc
attggagagactaattcccacagttcccttgagactcaactatattcactgggtagaagatctgatcggtcaccaggattctgacaaaagt
actctccgaagaggaattgacataggcacgggggcatcttgcatctaccccttacttggagcaaccttgaatggctggtatttcctcgca
acagaagtggatgatatgtgtttcaactatgcaaagaaaaatgtggaacagaataacttatctgatctcataaaagtggtgaaagtgcca
cagaagacactcctgatggatgctcttaaagaagaatctgagataatctatgacttttgcatgtgcaaccctcccttttttgccaatcaattg
gaagccaagggagtaaactcacgaaatcctcgaagacctccgcctagttctgttaatacaggaggcatcacagagatcatggcagaa
ggaggtgaattagagtttgttaaaaggatcatccatgacagtctacaacttaaaaaaagattaagatggtatagctgcatgctgggaaag
aaatgcagcctggcgcctctgaaggaggagcttcgcatacaaggggttcccaaagtaacgtacactgaattctgtcaaggtcggacaa
tgagatgggccttagcttggagtttttatgatgatgtcacagtaccatcaccaccaagtaagcgaagaaaattagagaaaccgagaaaa
cccataacattcgtggtgctggcgtccgtgatgaaggaattatccctcaaagcatcacctctgcgctcggagacggcggaaggcatag
tcgttgtcacgacatggattgaaaaaattctcactgatttgaaggtccagcataaacgagttccctgtggaaaagaggaagtcagcctttt
cctaacggccatagaaaactcctggattcatttaaggagaaagaaaagagagcgtgtgagacagctgagagaagttccccgagctcc
tgaggacgtcattcaggccttggaagagaaaaagcccacccccaaagagtctggcaatagccaagaactggccaggggcccccag
gagaggaccccctgtgggcctgctctgcgggaaggcgaggctgccgctgtggagggcccgtgcccgagccaggagtccctgtcc
caggaggaaaacccggaacccacggaggatgaaaggagtgaggaaaagggaggggtggaggttttggaaagttgtcaaggctct
agcaacggagcccaggaccaagaggcttctgagcagttcggcagcccagtggctgaaagggggaaacgtctcccaggagtggcc
ggacagtacctgtttaagtgtttgataaacgttaagaaggaggtggacgatgccttagtggagatgcactgggttgagggccagaaca
gggatctgatgaaccagctttgcacctacatacgtaaccaaattttcaggcttgttgcagttaactagaaacctcctgcacagttggaaac
gtgttgatagtaacttgctttggagtggcctgtggggtggcaagaggaatcctaccagcggcccattagtagcacgatgtggaattatct
tcgaaaacaaaaacctatgaatctgtcccccacctccccccgcctccttcccgctttttgagttacagggagtcgtagtgtggtcatttaca
aggaggaattgtggtcatcagtaacaacagaaagccctcagtaaactcccgagggattgcaagctggctcaagctggcccctcagct
ctggactgcctctgcaaggtcagaagggttgtttgtggagtctgggctgggcagcactgcctagaatatcatgctgtctctgtcacccaa
gggtgtttcttgaggaggggtggctctctctgcctccagctggaggccctggtaccctgttctaggtcactcttcaagatggggcctacc
ttgcatcaatcccacaaagggagctgtatggtgggtggtggggaatctgggagagaaaccttagtaatgctgggaaggagcagcaga
gtctggggaccacccggtaaatggcacattcctgacacctggctgttttgatgttgcttatttcagaagcagaattaggtaagcaaaactc
cccggtgtgactgaggcacacagaaggcacccatacccccacctccagcctgttgacagtaccattttgtagcagttttactactgtgtg
atttttgtttggacatctgaagtagagcttgttttgtttttaaataagaatattcacaaattaaaaaccagcggtcctatttgaatcctggggtta
gctgagtgagcggctgatgatagaaatgagaaatagaacaaaatagtatgtgccgtaggtagcttaagaaagtctcagatattttgttgc
tgatcaaatactgtttttttgtggcttcacttgtaatcccccctgtacttacctactcacattggagagttctgaggccggagtaactgtgtcct
tgaaacacgtttctaattggaatgccagggttcagtagccgtccccccggaaaggggtgaccttttgctgtgcttgatgttgcatcagca
gcctagggttctgtttagactaaaatcttggccagagctccttgccatctgctaagaagactggggctgagtagttaagccagccttctga
gaggtggctgttggtcaggacgggaagctggtgaccttggcatgtcttggcagcagctagatcaggccctcggcagagacacagga
agcggaactgctgtgccttaacttggctgtggagctggagctggagaaggcagcatactgaccagtggctttttgattgattgtttgttat
gaggtggagttttactcttgttgtctaggctggagtgccgtggtgcgatcttagctcactgcaacccccgcctcccgggttcaagcgattc
tcctgcctcagcctcccaagtagctgggattacaggcacgcgccaccacgcctggctaattttgtgtttttggtagagatgggatttcacc
atgttggccaggctaatctcgaactcatgatctcgggtgatccgcccaccttggcctcccaaagtgctgggattacagccgtgagccac
tactcccagcctctgaccagtgttcttaacctggtccgtggacctccagagagtccatgtacctcctagagttacttctaaaagctctgtga
gcatgtgtgtgtgtgtgtgtgtgtgtgtgtattttttttcctggagagagggttcccagaaccctcagacacagacaaaggggtcaataac
ccactaaggattaagaatcattattctagtccaagcattcatgtgtcaggctgcaaaaaacaatacccagggtcacacagagccaagac
tcaattcaggaccgtggattcccctggtctagaaattttctgctgtgccagcccacaccaccccactgtccttacctcgagtgaatattaca
tttgagtcatttgctgggcccaaacctagtttccttggtataattttaggataattgtttaagtggcaactattcattcagtaagtagtaagtact
tattgtttgcttgtttcattatgaaagagtggcacatgctcattaaagatttggaaaaatgaaagtcaaaacaacaaaatcaccccgagtcc
caaccttctgtaacataaccactcttggcattggcgtgttcctttctagtctctctgtagacggggtgtgtgagtgtgtgggtttaactttggtt
gtcctcatgctgcgtattcagttttgtattctggtcctttgttcatttaacatcttacaagtatttgtccatgttgtaacagtagtgtattagctt
acactccttgcctgttcaaaatgtctttcaggcacagcactggcctttaagcctgtgtcgtagggatttccagagaatgctctgtgtattgaag
cacagaaggtgtttctgtgtctcagtgtgtttctgtccctaggtttaaggcttcatgtcatggaggagatntatagatgtcaagctaatgacc
ttagagttttaaaaaatccgtgaccgtggccaggcgcagtggctcacgcctgtaatcccagcactgtgaggctgagatgggcgcatcg
catgaggtcgggagtttgagaccagcctggccaacatggcgaaaccccgtctctactcaaaatacaaaaattagccgggcatgatag
cacgtgcctgtaatcccagctactcgggaggctgaggcaggagactcgcttgaacctgggaggtggaggttgcagtgagccgagaa
ccagctttcagtctggagccgagtgccttctgtgcatttggatgtttccatttccttccctgagaagattttcttaggctacctagtgagaga
acattgaaaatatttttaaaggacatctaagcattgttttggtcatgcatatgctttataattgtgtgttgtttcatagcatatacctctggtaca
ggtgggcaagtttttctttgaagaaatgggttattgactcatatgtcataaccttgagtgttactctcccggtgtccagaggtcacattcatgtt
gcggggttggtatgaaattaaatcttggtgatgtgaccctacattctcttctggtccctagaatcggcttctggtctcctgataactgaagtg
gagacagaagttgagcctgttgcccaggcaaactaaagctgcttttgttcttcggaatctgctttgcctccgtcagcctgcttccttcccca
cacatgctggccgcactgtccccactccagacctctgctgtgtgtcctgggcagggccgcgttttggcagtaccctttcaactcatccta
agcttcgtgtagattactttagtatatattttttataaaacataaagcctttcctctcgatggaaatcaaagcttaccatgtgagcactcgaact
tctaagttgtgacaggaataacaaaactgcaaggagtggaaaagatggaaaagcctgtgggaaatccgaggccttttgaaagaaggg
agctgatgacttcacgaccagctcctggagcccctcctttctgctgaagccgcggcatttccctccgtggccacacgagggcacccttg
gcccttttatcaaagcgccttcacttccccgtgggaatggagacaagtctgtccacggtgttttcttgaaatacccagttgctacccagatt
tgtatttttatgtaaacaaatacattttcacagaaataaaatttgaaaaataaaagtagaaagagaaaaaaa
WTAP

| FEATURES | Location/Qualifiers |
|---|---|
| source | 1..2133 |
| | /organism = ″Homo sapiens″ |
| | /mol_type = ″mRNA″ |
| | /db_xref = ″taxon:9606″ |
| | /chromosome = ″6″ |
| | /map = ″6q25.3″ |
| gene | 1..2133 |
| | /gene = ″WTAP″ |
| | /gene_synonym = ″Mum2″ |
| | /note = ″WT1 associated protein″ |
| | /db_xref = ″GeneID:9589″ |
| | /db_xref = ″HGNC:HGNC:16846″ |
| | /db_xref = ″MIM:605442″ |
| exon | 1..204 |
| | /gene = ″WTAP″ |
| | /gene_synonym = ″Mum2″ |
| | /inference = ″alignment:Splign:2.1.0″ |
| misc_feature | 75..77 |
| | /gene = ″WTAP″ |
| | /gene_synonym = ″Mum2″ |
| | /note = ″upstream in-frame stop codon″ |
| exon | 205..242 |
| | /gene = ″WTAP″ |
| | /gene_synonym = ″Mum2″ |
| | /inference = ″alignment:Splign:2.1.0″ |
| CDS | 213..1403 |
| | /gene = ″WTAP″ |
| | /gene_synonym = ″Mum2″ |
| | /note = ″isoform 1 is encoded by transcript variant 4; |
| | Wilms' tumour 1-associating protein; PNAS-132; putative |
| | pre-mRNA splicing regulator female-lethal(2D); |
| | pre-mRNA-splicing regulator WTAP; hFL(2)D; |
| | female-lethal(2)D homolog; wilms tumor 1-associating |
| | protein; Wilms tumor 1 associated protein″ |
| | /codon start = 1 |
| | /product = ″pre-mRNA-splicing regulator WTAP isoform 1″ |
| | /protein_id = ″NP_001257460.1″ |
| | /db_xref = ″CCDS:CCDS5266.1″ |
| | /db_xref = ″GeneID:9589″ |
| | /db_xref = ″HGNC:HGNC:16846″ |
| | /db_xref = ″MIM:605442″ |

/translation = ″MTNEEPLPKKVRLSETDFKVMARDELILRWKQYEAVVQALEGKY
TDLNSNDVTGLRESEEKLKQQQQESARRENILVMRLATKEQEMQECTTQIQYLKQV
QQ
PSVAQLRSTMVDPAINLFFLKMKGELEQTKDKLEQAQNELSAWKFTPDSQTGKKLM
AK
CRMLIQENQELGRQLSQGRIAQLEAELALQKKYSEELKSSQDELNDFIIQLDEEVEGM
QSTILVLQQQLKETRQQLAQYQQQQSQASAPSTSRTTASEPVEQSEATSKDCSRLTN
G
PSNGSSSRQRTSGSGFHREGNTTEDDFPSSPGNGNKSSNSSEERTGRGGSGYVNQLSA
GYESVDSPTGSENSLTHQSNDTDSSHDPQEEKAVSGKGNRTVGSRHVQNGLDSSVN
VQ
GSVL″

| | |
|---|---|
| misc_feature | 213..215 |
| | /gene = ″WTAP″ |
| | /gene_synonym = ″Mum2″ |
| | /experiment = ″experimental evidence, no additional details |
| | recorded″ |
| | /note = ″N-acetylmethionine. {ECO:0000244\|PubMed:22814378, |
| | ECO:0000269\|Ref.7}; propagated from UniProtKB/Swiss-Prot |
| | (Q15007.2); acetylation site″ |
| misc_feature | 252..254 |
| | /gene = ″WTAP″ |
| | /gene_synonym = ″Mum2″ |
| | /experiment = ″experimental evidence, no additional details |
| | recorded″ |
| | /note = ″Phosphoserine. {ECO:0000244\|PubMed:23186163}; |
| | propagated from UniProtKB/Swiss-Prot (Q15007.2); |
| | phosphorylation site″ |
| misc_feature | 1125..1127 |
| | /gene = ″WTAP″ |
| | /gene_synonym = ″Mum2″ |
| | /experiment = ″experimental evidence, no additional details |
| | recorded″ |
| | /note = ″Phosphoserine. {ECO:0000244}PubMed:19690332, |
| | ECO:0000244}PubMed:23186163}; propagated from |
| | UniProtKB/Swiss-Prot (Q15007.2); phosphorylation site″ |
| misc_feature | 1128..1130 |
| | /gene = ″WTAP″ |
| | /gene_synonym = ″Mum2″ |
| | /experiment = ″experimental evidence, no additional details |
| | recorded″ |
| | /note = ″Phosphoserine. {ECO:0000244\|PubMed:18669648, |
| | ECO:0000244\|PubMed:19690332, ECO:0000244\|PubMed:20068231, |
| | ECO:0000244\|PubMed:21406692}; propagated from |
| | UniProtKB/Swiss-Prot (Q15007.2); phosphorylation site″ |
| misc_feature | 1233..1235 |
| | /gene = ″WTAP″ |
| | /gene_synonym = ″Mum2″ |
| | /experiment = ″experimental evidence, no additional details |
| | recorded″ |
| | /note = ″Phosphoserine. {ECO:0000250\|UniProtKB:Q9ER69}; |
| | propagated from UniProtKB/Swiss-Prot (Q15007.2); |
| | phosphorylation site″ |
| misc_feature | 1260..1262 |
| | /gene = ″WTAP″ |
| | /gene_synonym = ″Mum2″ |
| | /experiment = ″experimental evidence, no additional details |
| | recorded″ |
| | /note = ″Phosphothreonine. {ECO:0000250\|UniProtKB:Q9ER69}; |
| | propagated from UniProtKB/Swiss-Prot (Q15007.2); |
| | phosphorylation site″ |
| misc_feature | 1374..1376 |
| | /gene = ″WTAP″ |
| | /gene_synonym = ″Mum2″ |
| | /experiment = ″experimental evidence, no additional details |
| | recorded″ |
| | /note = ″Phosphoserine. {ECO:0000244\|PubMed:20068231}; |
| | propagated from UniProtKB/Swiss-Prot (Q15007.2); |
| | phosphorylation site″ |
| exon | 243..298 |
| | /gene = ″WTAP″ |
| | /gene_synonym = ″Mum2″ |
| | /inference = ″alignment:Splign:2.1.0″ |
| exon | 299..357 |
| | /gene = ″WTAP″ |
| | /gene_synonym = ″Mum2″ |
| | /inference = ″alignment:Splign:2.1.0″ |
| exon | 358..485 |
| | /gene = ″WTAP″ |
| | /gene_synonym = ″Mum2″ |
| | /inference = ″alignment:Splign:2.1.0″ |
| exon | 486..664 |
| | /gene = ″WTAP″ |
| | /gene_synonym = ″Mum2″ |
| | /inference = ″alignment:Splign:2.1.0″ |
| STS | 636..1362 |
| | /gene = ″WTAP″ |
| | /gene_synonym = ″Mum2″ |
| | /standard_name = ″Wtap″ |
| | /db_xref = ″UniSTS:498921″ |
| exon | 665..819 |
| | /gene = ″WTAP″ |
| | /gene_synonym = ″Mum2″ |
| | /inference = ″alignment:Splign:2.1.0″ |
| STS | 751..1054 |
| | /gene = ″WTAP″ |
| | /gene_synonym = ″Mum2″ |
| | /standard name = ″MARC_17739-17740:1031760457:1″ |
| | /db_xref = ″UniSTS:268391″ |
| exon | 820..2111 |
| | /gene = ″WTAP″ |
| | /gene_synonym = ″Mum2″ |
| | /inference = ″alignment:Splign:2.1.0″ |
| STS | 1597..1825 |
| | /gene = ″WTAP″ |
| | /gene_synonym = ″Mum2″ |
| | /standard_name = ″RH45141″ |
| | /db_xref = ″UniSTS:48858″ |
| regulatory | 2084..2089 |
| | /regulatory_class = ″polyA_signal_sequence″ |
| | /gene = ″WTAP″ |
| | /gene_synonym = ″Mum2″ |
| polyA_site | 2111 |
| | /gene = ″WTAP″ |
| | /gene_synonym = ″Mum2″ |

cDNA
ggtttcctccctcagcgccattttgtggcagcgagacccacaaataaaggggagcgcaggggttgcggcgggactaggagcgcgg
cggggccggcggcagagctgtccggctgcgcggtggcccggggggcccgggcggcagggcaagcagcgcggcctcggcctat
gcgaccggtggcgccggcgcggcttctgcctggagaggattcaagatgaccaacgaagaacctcttcccaagaaggttcgattgagt
gaaacagacttcaaagttatggcaagagatgagttaattctaagatggaaacaatatgaagcatatgtacaagctttggagggcaagta
cacagatcttaactctaatgatgtaactggcctaagagagtctgaagaaaaactaaagcaacaacagcaggagtctgcacgcaggga
aaacatccttgtaatgcgactagcaaccaaggaacaagagatgcaagagtgtactactcaaatccagtacctcaagcaagtccagcag
ccgagcgttgcccaactgagatcaacaatggtagacccagcgatcaacttgtttttcctaaaaatgaaaggtgaactggaacagactaa
agacaaactggaacaagcccaaaatgaactgagtgcctggaagtttacgcctgatagccaaacagggaaaaagttaatggcgaagtg
tcgaatgcttatccaggagaatcaagagcttggaaggcagctgtcccagggacgtattgcacaacttgaagcagagttggctttacaga
agaaatacagtgaggagcttaaaagcagtcaggatgaactgaatgacttcatcatccagcttgatgaagaagtagagggtatgcagag
taccattctagttctgcagcagcagctgaaggagacacgccagcagttggctcagtaccagcagcagcagtctcaggcctctgcccc
aagtaccagcaggactacagcttctgaacctgtagaacagtcagaggccacaagtaaagactgcagtcgtctgacaaacggaccaa
gtaatggtagctcctcccgccagaggacgtctgggtctggatttcacagggagggcaacacaaccgaagatgactttccttcttctcca
gggaatggtaataagtcctccaacagctcagaggagagaactggcagaggaggtagtggttacgtaaatcaactcagtgcggggtat
gaaagtgtagactctcccacgggcagtgaaaactctctcacacaccaatcaaatgacacagactccagtcatgaccctcaagaggag
aaagcagtgagtgggaaaggtaatcgaactgtgggttcccgccacgttcagaatggcttggactcaagtgtaaatgtacagggttcagt
tttgtaatattttttcagcaaatttttatacagtgtcatttaatttgggagaggatactgtccagaaaattaatgcatacttttgtcacaatttg
cctttttgtgggtgtacgttttggtttttttttgttgttttttttctttgttttuttttcttttctttttttttttttttttttttttttgcttc
aatacttctgccgctttggaaattgtaacagttaattactttgaatgttgctaaaaggacattttgtgtagggtcaagttatttttatatgagtt
aatgtgaaattgtaaatggaaatttttccttaaaatacaacacaatgatgtctgtataaatctgtctgtttagaatctgtgctgtgtaagggcat
tcgtactcatgctgttactgtacttatgcaccattcagacttgttagagtagatgtgggtttatgactgccaagtttgcccagtacagtagtttt
ttatcactaaaagttggactcattgatggagtcctgtagtagtttcagtgttagatacagttttttccaccatacatctgtgcattttctcttta
ggtgactgtttaagaaatttgtgtgcatagttactcagttntatgaactgttgtatcctgttaatgcatattgctctgtgactccagtatatctt
acctgtactgaccaaacctaaataaagatttttattgtaactccttaaaaaaaaaaaaaaaaaaaaaaaa
FTO

| FEATURES | Location/Qualifiers |
|---|---|
| source | 1..4313 |
| | /organism = ″Homo sapiens″ |
| | /mol_type = ″mRNA″ |
| | /db_xref = ″taxon:9606″ |
| | /chromosome = ″16″ |
| | /map = ″16q12.2″ |
| gene | 1..4313 |
| | /gene = ″FTO″ |
| | /gene_synonym = ″ALKBH9; BMIQ14; GDFD″ |
| | /note = ″FTO, alpha-ketoglutarate dependent dioxygenase″ |
| | /db_xref = ″GeneID:79068″ |
| | /db_xref = ″HGNC:HGNC:24678″ |
| | /db_xref = ″MIM:610966″ |
| exon | 1..267 |
| | /gene = ″FTO″ |
| | /gene_synonym = ″ALKBH9; BMIQ14; GDFD″ |
| | /inference = ″alignment:Splign:2.1.0″ |
| misc_feature | 43..45 |
| | /gene = ″FTO″ |
| | /gene_synonym = ″ALKBH9; BMIQ14; GDFD″ |
| | /note = ″upstream in-frame stop codon″ |
| CDS | 223..1740 |
| | /gene = ″FTO″ |
| | /gene_synonym = ″ALKBH9; BMIQ14; GDFD″ |
| | /EC_number = ″1.14.11.-″ |
| | /note = ″isoform 3 is encoded by transcript variant 3; |
| | alpha-ketoglutarate-dependent dioxygenase FTO; fat mass |
| | and obesity-associated protein; AlkB homolog 9; fat mass |
| | and obesity associated″ |
| | /codon_start = 1 |
| | /product = ″alpha-ketoglutarate-dependent dioxygenase FTO |
| | isoform 3″ |
| | /protein_id = ″NP_001073901.1″ |
| | /db_xref = ″CCDS:CCDS32448.1″ |
| | /db_xref = ″GeneID:79068″ |
| | /db_xref = ″HGNC:HGNC:24678″ |
| | /db_xref = ″MIM:610966″ |

/translation = ″MKRTPTAEEREREAKKLRLLEELEDTWLPYLTPKDDEFYQQWQL
KYPKLILREASSVSEELHKEVQEAFLTLHKHGCLFRDLVRIQGKDLLTPVSRILIGNP
GCTYKYLNTRLFTVPWPVKGSNIKHTEAEIAAACETFLKLNDYLQIETIQALEELAAK
EKANEDAVPLCMSADFPRVGMGSSYNGQDEVDIKSRAAYNVTLLNFMDPQKMPYL
KEE
PYFGMGKMAVSWHHDENLVDRSAVAVYSYSCEGPEEESEDDSHLEGRDPDIWHVG
FM
SWDIETPGLAIPLHQGDCYFMLDDLNATHQHCVLAGSQPRFSSTHRVAECSTGTLDY
I
LQRCQLALQNVCDDVDNDDVSLKSFEPAVLKQGEEIHNEVEFEWLRQFWFQGNRY
RKC
TDWWCQPMAQLEALWKKMEGVTNAVLHEVKREGLPVEQRNEILTAILASLTARQN
LRR
EWHARCQSRIARTLPADQKPECRPYWEKDDASMPLPFDLTDIVSELRGQLLEAKP″

| | |
|---|---|
| misc_feature | 232..234 |
| | /gene = ″FTO″ |
| | /gene_synonym = ″ALKBH9; BMIQ14; GDFD″ |
| | /experiment = ″experimental evidence, no additional details |
| | recorded″ |
| | /note = ″Phosphothreonine. {ECO:0000244\|PubMed:23186163}; |
| | propagated from UniProtKB/Swiss-Prot (Q9C0B1.3); |
| | phosphorylation site″ |
| misc_feature | 316..1203 |
| | /gene = ″FTO″ |
| | /gene_synonym = ″ALKBH9; BMIQ14; GDFD″ |
| | /experiment = ″experimental evidence, no additional details |
| | recorded″ |
| | /note = ″propagated from UniProtKB/Swiss-Prot (Q9C0B1.3); |
| | Region: Fe2OG dioxygenase domain″ |
| misc_feature | 859..894 |
| | /gene = ″FTO″ |
| | /gene_synonym = ″ALKBH9; BMIQ14; GDFD″ |
| | /experiment = ″experimental evidence, no additional details |
| | recorded″ |
| | /note = ″propagated from UniProtKB/Swiss-Prot (Q9C0B1.3); |
| | Region: Loop L1, predicted to block binding of |
| | double-stranded DNA or RNA″ |
| misc_feature | 868..870 |
| | /gene = ″FTO″ |
| | /gene_synonym = ″ALKBH9; BMIQ14; GDFD″ |
| | /experiment = ″experimental evidence, no additional details |
| | recorded″ |
| | /note = ″N6-acetyllysine. {ECO:0000244\|PubMed:19608861}; |
| | propagated from UniProtKB/Swiss-Prot (Q9C0B1.3); |
| | acetylation site″ |
| misc_feature | 913..924 |
| | /gene = ″FTO″ |
| | /gene_synonym = ″ALKBH9; BMIQ14; GDFD″ |
| | /experiment = ″experimental evidence, no additional details |
| | recorded″ |
| | /note = ″propagated from UniProtKB/Swiss-Prot (Q9C0B1.3); |
| | Region: Substrate binding″ |
| misc_feature | 1168..1176 |
| | /gene = ″FTO″ |
| | /gene_synonym = ″ALKBH9; BMIQ14; GDFD″ |
| | /experiment = ″experimental evidence, no additional details |
| | recorded″ |
| | /note = ″propagated from UniProtKB/Swiss-Prot (Q9C0B1.3); |
| | Region: Alpha-ketoglutarate binding″ |
| exon | 268..345 |
| | /gene = ″FTO″ |
| | /gene_synonym = ″ALKBH9; BMIQ14; GDFD″ |
| | /inference = ″alignment:Splign:2.1.0″ |
| exon | 346..973 |
| | /gene = ″FTO″ |
| | /gene_synonym = ″ALKBH9; BMIQ14; GDFD″ |
| | /inference = ″alignment:Splign:2.1.0″ |
| exon | 974..1117 |
| | /gene = ″FTO″ |
| | /gene_synonym = ″ALKBH9; BMIQ14; GDFD″ |
| | /inference = ″alignment:Splign:2.1.0″ |
| exon | 1118..1197 |
| | /gene = ″FTO″ |
| | /gene_synonym = ″ALKBH9; BMIQ14; GDFD″ |
| | /inference = ″alignment:Splign:2.1.0″ |
| exon | 1198..1341 |
| | /gene = ″FTO″ |
| | /gene_synonym = ″ALKBH9; BMIQ14; GDFD″ |
| | /inference = ″alignment:Splign:2.1.0″ |
| exon | 1342..1461 |
| | /gene = ″FTO″ |
| | /gene_synonym = ″ALKBH9; BMIQ14; GDFD″ |
| | /inference = ″alignment:Splign:2.1.0″ |
| exon | 1462..1586 |
| | /gene = ″FTO″ |
| | /gene_synonym = ″ALKBH9; BMIQ14; GDFD″ |
| | /inference = ″alignment:Splign:2.1.0″ |
| exon | 1587..4292 |
| | /gene = ″FTO″ |
| | /gene_synonym = ″ALKBH9; BMIQ14; GDFD″ |
| | /inference = ″alignment:Splign:2.1.0″ |
| STS | 3072..3202 |
| | /gene = ″FTO″ |
| | /gene_synonym = ″ALKBH9; BMIQ14; GDFD″ |
| | /standard_name = ″SHGC-60773″ |
| | /db_xref = ″UniSTS:27100″ |
| regulatory | 3205..3210 |
| | /regulatory_class = ″polyA_signal_sequence″ |
| | /gene = ″FTO″ |
| | /gene_synonym = ″ALKBH9; BMIQ14; GDFD″ |
| polyA_site | 3229 |
| | /gene = ″FTO″ |
| | /gene_synonym = ″ALKBH9; BMIQ14; GDFD″ |
| STS | 3337..3500 |
| | /gene = ″FTO″ |
| | /gene_synonym = ″ALKBH9; BMIQ14; GDFD″ |
| | /standard_name = ″RH48882″ |
| | /db_xref = ″UniSTS:58061″ |
| STS | 3705..3774 |
| | /gene = ″FTO″ |
| | /gene_synonym = ″ALKBH9; BMIQ14; GDFD″ |
| | /standard_name = ″D1S1423″ |
| | /db_xref = ″UniSTS:149619″ |
| STS | 3963..4239 |
| | /gene = ″FTO″ |
| | /gene_synonym = ″ALKBH9; BMIQ14; GDFD″ |
| | /standard_name = ″D16S2971″ |
| | /db_xref = ″UniSTS:19408″ |
| STS | 4056..4204 |
| | /gene = ″FTO″ |
| | /gene_synonym = ″ALKBH9; BMIQ14; GDFD″ |
| | /standard_name = ″D1652577E″ |
| | /db_xref = ″UniSTS:45130″ |
| regulatory | 4258..4263 |
| | /regulatory class = ″polyA_signal_sequence″ |
| | /gene = ″FTO″ |
| | /gene_synonym = ″ALKBH9; BMIQ14; GDFD″ |
| polyA_site | 4292 |
| | /gene = ″FTO″ |
| | /gene_synonym = ″ALKBH9; BMIQ14; GDFD″ |

cDNA
ctacgctcttccagctgtcggacctgggaaattctcctgtgctaaatcccgtggcgctcgcgggtgtcgccgcggtgcatcctgggagt
tgtagttttttctactcagagggagaatagctccagacgggagcaggacgctgagagaactacatgcaggaggcggggtccagggc
gagggatctacgcagcttgcggtggcgaaggcggctttagtggcagcatgaagcgcaccccgactgccgaggaacgagagcgcg
aagctaagaaactgaggcttcttgaagagcttgaagacacttggctcccttatctgacccccaaagatgatgaattctatcagcagtggc
agctgaaatatcctaaactaattctccgagaagccagcagtgtatctgaggagctccataaagaggttcaagaagcctttctcacactgc
acaagcatggctgcttatttcgggacctggttaggatccaaggcaaagatctgctcactccggtatctcgcatcctcattggtaatccag
gctgcacctacaagtacctgaacaccaggctctttacggtcccctggccagtgaaagggtctaatataaaacacaccgaggctgaaat
agccgctgcttgtgagaccttcctcaagctcaatgactacctgcagatagaaaccatccaggctttggaagaacttgctgccaaagaga
aggctaatgaggatgctgtgccattgtgtatgtctgcagatttccccagggttgggatgggttcatcctacaacggacaagatgaagtgg
acattaagagcagagcagcatacaacgtaactttgctgaatttcatggatcctcagaaaatgccatacctgaaagaggaaccttattttgg
catggggaaaatggcagtgagctggcatcatgatgaaaatctggtggacaggtcagcggtggcagtgtacagttatagctgtgaagg
ccctgaagaggaaagtgaggatgactctcatctcgaaggcagggatcctgatatttggcatgttggttttaagatctcatgggacataga
gacacctggtttggcgataccccttcaccaaggagactgctatttcatgcttgatgatctcaatgccacccaccaacactgtgttttggcc
ggttcacaacctcggtttagttccacccaccgagtggcagagtgctcaacaggaaccttggattatattttacaacgctgtcagttggctc
tgcagaatgtctgtgacgatgtggacaatgatgatgtctctttgaaatcctttgagcctgcagttttgaaacaaggagaagaaattcataat
gaggtcgagtttgagtggctgaggcagttttggtttcaaggcaatcgatacagaaagtgcactgactggtggtgtcaacccatggctca
actggaagcactgtggaagaagatggagggtgtgacaaatgctgtgcttcatgaagttaaaagagaggggctccccgtggaacaaa
ggaatgaaatcttgactgccatccttgcctcgctcactgcacgccagaacctgaggagagaatggcatgccaggtgccagtcacgaat
tgcccgaacattacctgctgatcagaagccagaatgtcggccatactgggaaaaggatgatgcttcgatgcctctgccgtttgacctca
cagacatcgtttcagaactcagaggtcagcttctggaagcaaaaccctagaaggagcacaagtctcaggcggaggagaaaaagaga
tcggcttttctcctccaacgttgtcatgggcttaagcaagagcagtggagacttctcttggcccctagattgtagcacccgggtcccaatc
caaaacagctaggaaatggtgcccatgaagttttaaatgttttaaaatgaccctgtgttatagtctgatttggtgttaaacaggaccttcttc
ccccaaaattgttcagattataaaatgtgagccattcagcccccaaggtccagggcaggcgacaggaacgagcccagcgtgtgacaa
agcctaacctactttcctctttcccaagctttttcagagactctggagtggacccagccctctggggaaagacagaacttagagacatcc
cagttactcaccacacccatagtgctgtccaatatggtagccactagctagctgtggctacttcaatttaaattcagttttaattttaattaaaa
atgcagctcttcagtcgccctggccacatttcaagtgcttaacagcctcatgtggctagtgactgctgtattggacggtacagatatggaa
cattttcatcatcgaagaaagtcctattggacaacacttctataaaaagtttgagagcaggaattctcatttccattcgtctgtagcttctatc
cccaaaggcaaagaaactaaaagagaaatgactcattgaagattggcctctttcctttctctaagacaaacctaagtaaaagcctgagct
ttgagtcctatgctcagcacacgggaaggagatgttaataattaaaataaagttgatatcctgtctttagggagttcccttgatctcttgaaa
gagacacagccccatttacattatttcgtggatttcaccagcatagtatagtttttttctgtaagtccctcattcttatgtaataacaggtggaa
ctgaggtttgaagaacctcagtggcccatcctgatgacattggagactcaaagagacaagagagagtagggtttaaaacctgagcttta
agactcccactagcttcgtgtcctttggcatgttaacgtgcctcagtttcctcatctgtataatggggatatatgaaaggcaccagtcctaa
ggtgaacattaagtgagatgattctagttacagacttagaacaatttccagcacatagttaaatatccaggaaattctggtactgttatgtgt
gggtgagctgacctggatgtagatgttttcctctctcttgctgacccctccgccagttttgtcttgtgatgccattaacacatctctccctttct
gacctggctcctgcccattggtgtcccaagaaatcgtgagaatagttagccccccgtctccccagcctgttgctttctcgtgtagttgttca
cagtagttgagaagttgaagagcttttgcctattgaaggtgcactgagaataaactctttcctgccaccagaattgcagtggttcacggcc
tgcactcattcccatgaatgcagttaatagccacagaaatgtcacattaagcaaagcagccagggtctcatcgtgttgagactcgagtct
ctcagaccttggattcattccctggtgtctttgagcctcagtttcctcattggtaaaagagaagtgaagcagtgtctcacagggtcattaca
gagattaaatgaaataaatgaaataacatagaccaggagggcgtggtgtttaaaagtcacagatggggcaccctcgggccatccagc
ccagtgttttctttagcccctatgatgttcattttttgttatatcccattaggtgcccatatttaaaaattgggagatttcacataaaattaaaa
ggtctgcattttcttttttcttttctttttttttttttttgagacacagtctcactctgtcaccaggctagagtgcagtggcacgatctcagctc
actgcaacctctgcctcccaggttcaagtaattctcctgcctcagcctcccaagtagctgggactacaggcacgtgccaccacgcccagctaat
ttttgtatttttagcagagatggggtttcaccacattggccaggatggtctcgatctcaacctcgtgatccacccacctcggtctcccaaag
cgctgggattacaggcgtgagccaccgcgccaagccaaggtctgcatttttctttagaactcagaacacccaatagtcctaggccccc
atcctcgcatggcagcaagctaaataagcatcttcccactgcgagttggggcatgacccagcctatggtttgccatactccctctttttctc
cgttttttcattaattgtgaacctgacctgcatcaccctttcatgtcagtgctctccaaacctgcttgcttgcacccctctagtcgaaatattttg
tgcttaccccaatatatgtgtgtgactattgaactctattcgtagactgcttgtactaatgtcatttgcatcataaaatattcatatccaataaac
atattaaaaggatgagataagaaaccgaaaaaaaaaaaaaaaaaaaaaa
ALKBH5

| FEATURES | Location/Qualifiers |
|---|---|
| source | 1..3449 |
| | /organism = ″Homo sapiens″ |
| | /mol_type = ″mRNA″ |
| | /db_xref = ″taxon:9606″ |
| | /chromosome = ″17″ |
| | /map = ″17p11.2″ |
| gene | 1..3449 |
| | /gene = ″ALKBH5″ |
| | /gene_synonym = ″ABH5; OFOXD; OFOXD1″ |
| | /note = ″alkB homolog 5, RNA demethylase″ |
| | /db_xref = ″GeneID:54890″ |
| | /db_xref = ″HGNC:HGNC:25996″ |
| | /db_xref = ″MIM:613303″ |
| exon | 1..1461 |
| | /gene = ″ALKBH5″ |
| | /gene_synonym = ″ABH5; OFOXD; OFOXD1″ |
| | /inference = ″alignment:Splign:2.1.0″ |
| misc_feature | 671..673 |
| | /gene = ″ALKBH5″ |
| | /gene_synonym = ″ABH5; OFOXD; OFOXD1″ |
| | /note = ″upstream in-frame stop codon″ |
| CDS | 692..1876 |
| | /gene = ″ALKBH5″ |
| | /gene_synonym = ″ABH5; OFOXD; OFOXD1″ |
| | /EC_number = ″1.14.11.-″ |
| | /note = ″oxoglutarate and iron-dependent oxygenase domain |
| | containing; alpha-ketoglutarate-dependent dioxygenase alkB |
| | homolog 5; alkB, alkylation repair homolog 5; alkylated |
| | DNA repair protein alkB homolog 5; probable |
| | alpha-ketoglutarate-dependent dioxygenase ABH5; AlkB |
| | family member 5, RNA demethylase″ |
| | /codon_start = 1 |
| | /product = ″RNA demethylase ALKBH5″ |
| | /protein_id = ″NP_060228.3″ |
| | /db_xref = ″CCDS:CCDS42272.1″ |
| | /db_xref = ″GeneID:54890″ |
| | /db_xref = ″HGNC:HGNC:25996″ |
| | /db_xref = ″MIM:613303″ |

/translation = ″MAAASGYTDLREKLKSMTSRDNYKAGSREAAAAAAAAVAAAAAA
AAAAEPYPVSGAKRKYQEDSDPERSDYEEQQLQKEEEARKVKSGIRQMRLFSQDEC
AK
IEARIDEVVSRAEKGLYNEHTVDRAPLRNKYFFGEGYTYGAQLQKRGPGQERLYPPG
D
VDEIPEWVHQLVIQKLVEHRVIPEGFVNSAVINDYQPGGCIVSHVDPIHIFERPIVSV
SFFSDSALCFGCKFQFKPIRVSEPVLSLPVRRGSVTVLSGYAADEITHCIRPQDIKER
RAVIILRKTRLDAPRLETKSLSSSVLPPSYASDRLSGNNRDPALKPKRSHRKADPDAA
HRPRILEMDKEENRRSVLLPTHRRRGSFSSENYWRKSYESSEDCSEAAGSPARKVKM
R
RH″

| | |
|---|---|
| misc_feature | 695..697 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″N-acetylalanine. {ECO:0000244\|PubMed:19413330, |
| | ECO:0000244\|PubMed:22814378}; propagated from |
| | UniProtKB/Swiss-Prot (Q6P6C2.2); acetylation site″ |
| misc_feature | 881..883 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″Phosphoserine. {ECO:0000244\|PubMed:18669648, |
| | ECO:0000244\|PubMed:19690332, ECO:0000244\|PubMed:23186163, |
| | ECO:0000244\|PubMed:24275569}; propagated from |
| | UniProtKB/Swiss-Prot (Q6P6C2.2); phosphorylation site″ |
| misc_feature | 896..898 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″Phosphoserine. {ECO:0000244\|PubMed:18669648}; |
| | propagated from UniProtKB/Swiss-Prot (Q6P6C2.2); |
| | phosphorylation site″ |
| misc_feature | 902..904 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″Phosphotyrosine. {ECO:0000244\|PubMed:19690332}; |
| | propagated from UniProtKB/Swiss-Prot (Q6P6C2.2); |
| | phosphorylation site″ |
| misc_feature | 1085..1087 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″N6-acetyllysine. {ECO:0000244\|PubMed:19608861}; |
| | propagated from UniProtKB/Swiss-Prot (Q6P6C2.2); |
| | acetylation site″ |
| misc_feature | 1268..1276 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″propagated from UniProtKB/Swiss-Prot (Q6P6C2.2); |
| | Region: Alpha-ketoglutarate binding. |
| | {ECO:0000269\|PubMed:24778178}″ |
| misc_feature | 1766..1768 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″Omega-N-methylarginine. |
| | {ECO:0000244\|PubMed:24129315}; propagated from |
| | UniProtKB/Swiss-Prot (Q6P6C2.2); methylation site″ |
| misc_feature | 1772..1774 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″Phosphoserine. {ECO:0000244\|PubMed:18669648, |
| | ECO:0000244\|PubMed:23186163}; propagated from |
| | UniProtKB/Swiss-Prot (Q6P6C2.2); phosphorylation site″ |
| misc_feature | 1802..1804 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″Phosphoserine. {ECO:0000250\|UniProtKB:Q3TSG4}; |
| | propagated from UniProtKB/Swiss-Prot (Q6P6C2.2); |
| | phosphorylation site″ |
| misc_feature | 1811..1813 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″Phosphoserine. {ECO:0000244\|PubMed:19690332}; |
| | propagated from UniProtKB/Swiss-Prot (Q6P6C2.2); |
| | phosphorylation site″ |
| misc_feature | 1841..1843 |
| | /gene = ″ALKBH5″ |
| | /gene_synonym = ″ABH5; OFOXD; OFOXD1″ |
| | /experiment = ″experimental evidence, no additional details |
| | recorded″ |
| | /note = ″Phosphoserine. {ECO:0000250\|UniProtKB:Q3TSG4}; |
| | propagated from UniProtKB/Swiss-Prot (Q6P6C2.2); |
| | phosphorylation site″ |
| exon | 1462..1542 |
| | /gene = ″ALKBH5″ |
| | /gene_synonym = ″ABH5; OFOXD; OFOXD1″ |
| | /inference = ″alignment:Splign:2.1.0″ |
| exon | 1543..1698 |
| | /gene = ″ALKBH5″ |
| | /gene_synonym = ″ABH5; OFOXD; OFOXD1″ |
| | /inference = ″alignment:Splign:2.1.0″ |
| exon | 1699..3434 |
| | /gene = ″ALKBH5″ |
| | /gene_synonym = ″ABH5; OFOXD; OFOXD1″ |
| | /inference = ″alignment:Splign:2.1.0″ |
| STS | 2795..2995 |
| | /gene = ″ALKBH5″ |
| | /gene_synonym = ″ABH5; OFOXD; OFOXD1″ |
| | /standard_name = ″RH75515″ |
| | /db_xref = ″UniSTS:84097″ |
| STS | 3259..3408 |
| | /gene = ″ALKBH5″ |
| | /gene_synonym = ″ABH5; OFOXD; OFOXD1″ |
| | /standard_name = ″STS-H01962″ |
| | /db_xref = ″UniSTS:63662″ |

ORIGIN

| | |
|---|---|
| 1 | cggacgatgc cgtgacgcgg cacggcgaca ctgttggcaa tatgagcgca cccctgtaga |
| 61 | gggagccctt cggtcctgga ggcggcgcgg cgtgaagaca ggttgctatt tgagagcgtt |
| 121 | cccttgaagc ccctcagaga gtgggggagg ggcggcggac ggcaagcggt tcctgtctgc |
| 181 | gcttgcgccg gcgcctctgc cgacccggcc tgcacgcacg cgcatgcccg tagcgcgcgg |
| 241 | agccgcggtg gccggcagca ctgcgcgtgc gcggtgagga gcccgctaag gagcggcgct |
| 301 | ggcggacgtc gggctggctg cccgtgacgt cgtgcggaga gctttaaagt gcgggccggg |
| 361 | ccgggcgtcc gagggtctgg tcgggagtcg ggccgcgtct ccgcagcagc cctccgcggc |
| 421 | atgaggcgct gccggcgccc ctgccccgcg ggacgtggag aaggtggagg aggaagaagc |
| 481 | cccgttgtcg ccaccgttgc atgacccgcc gctcctgagg ccctacccca cgcccggacc |
| 541 | ctcgacgccc cccgccgggt cccccactca cgcatggggg ttcggcgcta aggacccccc |
| 601 | tccctccggg ggccccgggg cgcgtcccct tagagccatg cccggctgcc ccgcccgccc |
| 661 | cggaggaccc tagagcagcg tcgtgggggc catggcggcc gccagcggct acacggacct |
| 721 | gcgtgagaag ctcaagtcca tgacgtcccg ggacaactat aaggcgggca gccgggaggc |
| 781 | cgccgccgct gccgcagccg ccgtagccgc cgcagccgca gccgccgctg ccgccgaacc |
| 841 | ttaccctgtg tccggggcca agcgcaagta tcaggaggac tcggaccccg agcgcagcga |
| 901 | ctatgaggag cagcagctgc agaaggagga ggaggcgcgc aaggtgaaga gcggcatccg |
| 961 | ccagatgcgc ctcttcagcc aggacgagtg cgccaagatc gaggcccgca ttgacgaggt |
| 1021 | ggtgtcccgc gctgagaagg gcctgtacaa cgagcacacg gtggaccggg ccccactgcg |
| 1081 | caacaagtac ttcttcggcg aaggctacac ttacggcgcc cagctgcaga agcgcgggcc |
| 1141 | cggccaggag cgcctctacc cgccgggcga cgtggacgag atccccgagt gggtgcacca |
| 1201 | gctggtgatc caaaagctgg tggagcaccg cgtcatcccc gagggcttcg tcaacagcgc |
| 1261 | cgtcatcaac gactaccagc ccggcggctg catcgtgtct cacgtggacc ccatccacat |
| 1321 | cttcgagcgc cccatcgtgt ccgtgtcctt ctttagcgac tctgcgctgt gcttcggctg |
| 1381 | caagttccag ttcaagccta ttcgggtgtc ggaaccagtg ctttccctgc cggtgcgcag |
| 1441 | gggaagcgtg actgtgctca gtggatatgc tgctgatgaa atcactcact gcatacggcc |
| 1501 | tcaggacatc aaggagcgcc gagcagtcat catcctcagg aagacaagat tagatgcacc |
| 1561 | ccggttggaa acaaagtccc tgagcagctc cgtgttacca cccagctatg cttcagatcg |
| 1621 | cctgtcagga aacaacaggg accctgctct gaaacccaag cggtcccacc gcaaggcaga |
| 1681 | ccctgatgct gcccacaggc cacggatcct ggagatggac aaggaagaga accggcgctc |
| 1741 | ggtgctgctg cccacacacc ggcggagggg tagcttcagc tctgagaact actggcgcaa |
| 1801 | gtcatacgag tcctcagagg actgctctga ggcagcaggc agccctgccc gaaaggtgaa |
| 1861 | gatgcggcgg cactgagtct acccgccgcc ctcctgggaa ctctggctca tccttacgta |
| 1921 | gttgcccctc cttttgtttt gagggttttg tttttgttca ttggggggtt tttgtttttt |
| 1981 | gttttttgtt ttttttgatt ctatatattt ttccttggtt ttgttgcctg ttagggctga |
| 2041 | agaatagaat tggccaggac ctaggttctc atattcttgg tattcctcct ggatggaaag |
| 2101 | gctgttggca tcaatagggg acagaggctg atgctggagt ggccagtaga ggtggtggag |
| 2161 | cagagcagcc atcttttaag tggggctgta tcaggctggg tttatttaaa agcaacaaaa |
| 2221 | tgttttggtt aagaaaatta ttttgctttc agtgtaaatc ttcgcagtgt tctaaacaaa |
| 2281 | gttcagtctt ctgctcgccc ctttccctca ctgatgtctg cacttggttg aggtctcctg |
| 2341 | gagcctcaca ggctctgctg ttctccactt ctcacctgcc atccacgccc tgcaagctca |
| 2401 | tgcaaacacc ctttcttcct cctgcggcag agttgttcag gttgcctggg caggggctta |
| 2461 | aacagtgcca gcccctgcca tcccaaagct attgttaagc cccccaggcg tcctccaccc |
| 2521 | acgcccacta gcctgccatg tccacagttc cttgggctgc tgaggggcta gtgcagtggt |
| 2581 | cctgacctct cttatcaaga gcacacttct ttgctggttg ctccttttga gcatatgcgt |
| 2641 | gtgattattt ggaacagtta gacttgccac gttgggtcag ttttagaaat tgtttctagc |
| 2701 | tagagggact ggtgtccttc caagtctagc atttggggta tggaaaattg ttgtggtgtg |
| 2761 | tggtagggtt tttgttttct tttttgagtt ttttttcccc ctttagtctc ctggcttttt |
| 2821 | cctttccctt cccttctcca ctggccagct tgggcctcat cctcatgtca tccttctagg |
| 2881 | aaggcgcctg ccccatcttg tctgccggca gcatgcatcc aaggccagag ctcaggcctg |
| 2941 | cagactgggc tggtgcctcc tccgcttcag ggtatgggag ttggtgaagg ggctttcaaa |
| 3001 | aaataataag gaaaaaaagg taaagtcttt ggtagcttct atccactcag atcctggaag |
| 3061 | gcagcaaggt tttgtggatc tagattcatt aggaatgtct tcttgtcagc caggccagga |
| 3121 | cccgggcttg ccaagagcag aggccctccc agcaaccagg ataccaccac tttgggggct |
| 3181 | ttgtgtacag aggtccgggt ctgagacctc ataggctgca gaaatctggg gcagccacca |
| 3241 | tcaagaagcc cctctcaggg gccagaactc ctttgccagc gtggatttct caagtcggga |
| 3301 | ctgcataatt aaagcagttg cagttttatt ttttttacag cttttttccc aaaaatgatt |
| 3361 | tgtagttgtg tgtgcagcac ttcgccctga tatgtgtgct ctacaataaa aaccaaatct |
| 3421 | aatatatttt gaaaaaaaaa aaaaaaaaa |

1. A fusion protein comprising:
(i) a guide nucleotide sequence-programmable RNA binding protein; and
(ii) an effector enzyme.

2. The fusion protein of claim 1, wherein the effector enzyme is an RNA methylation modification protein (RMMP) or an enzyme with cytidine deaminase activity.

3. The fusion protein of claim 1, wherein the guide nucleotide sequence-programmable RNA binding protein is selected from: Cas9, modified Cas9, Cas13a, Cas13b, CasRX/Cas13d, and a biological equivalent of each thereof.

4. The fusion protein of claim 3, wherein the guide nucleotide sequence-programmable RNA binding protein is selected from: Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), Campylobacter jejuni Cas9 (CjeCas9), and Brevibacillus laterosporus Cas9 (BlatCas9).

5. (canceled)

6. The fusion protein of claim 1, further comprising a linker.

7. The fusion protein of claim 6, wherein the linker is a peptide linker.

8. (canceled)

9. The fusion protein of claim 6, wherein the linker is a non-peptide linker.

10.-16. (canceled)

17. The fusion protein of claim 1, wherein the guide nucleotide sequence-programmable RNA binding protein is bound to a guide RNA (gRNA), a crisprRNA (crRNA), or a trans-activating crRNA (tracrRNA).

18.-20. (canceled)

21. A polynucleotide encoding the fusion protein of claim 1.

22. A vector comprising the polynucleotide of claim 21, optionally wherein the vector is an adenoviral vector, an adeno-associated viral vector, or a lentiviral vector.

23.-26. (canceled)

27. A viral particle comprising the vector of claim 22.

28. A cell comprising the vector of claim 22.

29.-31. (canceled)

32. A system for modulating m⁶A RNA methylation of a target RNA, the system comprising:
(i) a fusion protein comprising (a) a guide nucleotide sequence-programmable RNA binding protein, and (b) an effector enzyme; and
(ii) a gRNA; or
(iii) a crRNA and a tracrRNA;
wherein the gRNA or the crRNA comprises a sequence complementary to a target RNA.

33. The system of claim 32, further comprising a PAMmer.

34. (canceled)

35. A method for modulating m⁶A RNA methylation of a target RNA, the method comprising contacting the target mRNA with the fusion protein of claim 1, wherein the guide nucleotide sequence-programmable RNA binding protein binds a gRNA or a crRNA that hybridizes to a region of the target RNA.

36. A method for modulating embryonic stem cell maintenance and/or differentiation, nervous system development, circadian rhythm, heat shock response, meiotic progression, DNA ultraviolet (UV) damage response, or XIST mediated gene silencing, the method comprising contacting a target mRNA with the fusion protein of claim 1, wherein the guide nucleotide sequence-programmable RNA binding protein binds a gRNA or a crRNA that hybridizes to a region of the target RNA.

37. A method for editing a cytidine base into a uridine base in a target RNA, the method comprising contacting the target RNA with the fusion protein of claim 1, wherein the guide nucleotide sequence-programmable RNA binding protein binds a gRNA or a crRNA that hybridizes to a region of the target RNA.

38.-44. (canceled)

45. A method for treating a disease or condition associated with m⁶A RNA methylation of a target RNA in a subject in need thereof, the method comprising administering a fusion protein comprising (i) a guide nucleotide sequence-programmable RNA binding protein, and (ii) an effector enzyme, a polynucleotide encoding a fusion protein comprising (i) a guide nucleotide sequence-programmable RNA binding protein, and (ii) an effector enzyme, a vector comprising a polypeptide encoding a fusion protein comprising (i) a guide nucleotide sequence-programmable RNA binding protein, and (ii) an effector enzyme, a viral particle comprising a vector comprising a polypeptide encoding a fusion protein comprising (i) a guide nucleotide sequence-programmable RNA binding protein, and (ii) an effector enzyme, or a cell comprising a vector comprising a polypeptide encoding a fusion protein comprising (i) a guide nucleotide sequence-programmable RNA binding protein to the subject, thereby treating the disease or condition associated with m⁶A RNA methylation.

46.-49. (canceled)

50. A kit comprising the fusion protein of claim 1 and optionally instructions for use.

51. (canceled)

52. A non-human transgenic animal comprising the fusion protein of claim 1.
